SEQUENCE LISTING (119 sequences)

SEQ ID NO: 1 - protein, 11 aa, Artificial Sequence
  CDRH1
GFSFSGNYYMC

SEQ ID NO: 2 - protein, 11 aa, Artificial Sequence
  CDRH1 variant
GFSFSGNYYMS

SEQ ID NO: 3 - protein, 18 aa, Artificial Sequence
  CDRH2
CLYTGSSGSTYYASWAKG

SEQ ID NO: 4 - protein, 18 aa, Artificial Sequence
  CDRH2 variant
SLYTGSSGSTYYASWAKG

SEQ ID NO: 5 - protein, 12 aa, Artificial Sequence
  CDRH3
DLGYEIDGYGGL

SEQ ID NO: 6 - protein, 12 aa, Artificial Sequence
  CDRH3 variant 1
DLGYEIDSYGGL

SEQ ID NO: 7 - protein, 12 aa, Artificial Sequence
  CDRH3 variant 2
DLGYEIDAYGGL

SEQ ID NO: 8 - protein, 12 aa, Artificial Sequence
  CDRH3 variant 3
DLGYEIDTYGGL

SEQ ID NO: 9 - protein, 13 aa, Artificial Sequence
  CDRL1
QASQSVYNNNNLS

SEQ ID NO: 10 - protein, 13 aa, Artificial Sequence
  CDRL1 variant 1
QASQSVYNNNSLS

SEQ ID NO: 11 - protein, 13 aa, Artificial Sequence
  CDRL1 variant 2
QASQSVYNNNQLS

SEQ ID NO: 12 - protein, 13 aa, Artificial Sequence
  CDRL1 variant 3
QASQSVYNNNNLA

SEQ ID NO: 13 - protein, 7 aa, Artificial Sequence
  CDRL2
DASKLAS

SEQ ID NO: 14 - protein, 12 aa, Artificial Sequence
  CDRL3
LGGYYSSGWYFA

SEQ ID NO: 15 - protein, 112 aa, Artificial Sequence
  Rabbit antibody 4133 VL region
AQVLTQTPSPVSAVVGGTVSISCQASQSVYNNNNLSWYQQKPGQPPKLLIYDASKLASGVPSRFKGSGSGTQFTLTISGVQCDDAATYYCLGGYYSSGWYFAFGGGTKVVVK

SEQ ID NO: 16 - protein, 122 aa, Artificial Sequence
  Rabbit antibody 4133 VH region
QEQLVESGGGLVQPEGSLTLTCTASGFSFSGNYYMCWVRQAPGKGLEWIGCLYTGSSGSTYYASWAKGRFTISKTSSTTVTLQMTSLTAADTATYFCARDLGYEIDGYGGLWGQGTLVTVSS

SEQ ID NO: 17 - protein, 112 aa, Artificial Sequence
  4133 gL7 V-region - IGKV1D-13 framework
AQVLTQSPSSLSASVGDRVTITCQASQSVYNNNNLSWYQQKPGKAPKLLIYDASKLASGVPSRFSGSGSGTQFTLTISSLQPEDFATYYCLGGYYSSGWYFAFGGGTKVEIK

SEQ ID NO: 18 - protein, 112 aa, Artificial Sequence
  4133 gL1 V-region - IGKV1D-13 framework
AQVLTQSPSSLSASVGDRVTITCQASQSVYNNNNLAWYQQKPGKAPKLLIYDASKLASGVPSRFSGSGSGTQFTLTISSLQPEDFATYYCLGGYYSSGWYFAFGGGTKVEIK

SEQ ID NO: 19 - protein, 123 aa, Artificial Sequence
  4133 gH1 V-region - IGHV3-21 framework
EVQLVESGGGLVKPGGSLRLSCAASGFSFSGNYYMCWVRQAPGKGLEWIGCLYTGSSGSTYYASWAKGRFTISKDSAKTSVYLQMNSLRAEDTAVYYCARDLGYEIDGYGGLWGQGTLVTVSS

SEQ ID NO: 20 - protein, 123 aa, Artificial Sequence
  4133 gH4 V-region - IGHV3-21 framework
EVQLVESGGGLVKPGGSLRLSCAASGFSFSGNYYMSWVRQAPGKGLEWIGSLYTGSSGSTYYASWAKGRFTISKDSAKTSVYLQMNSLRAEDTAVYYCARDLGYEIDGYGGLWGQGTLVTVSS

SEQ ID NO: 21 - protein, 123 aa, Artificial Sequence
  4133 gH1 V-region - IGHV4-4 framework
EVQLQESGPGLVKPSGTLSLTCAASGFSFSGNYYMCWVRQPPGKGLEWIGCLYTGSSGSTYYASWAKGRVTISKDSSKTQVSLKLSSVTAADTAVYYCARDLGYEIDGYGGLWGQGTLVTVSS

SEQ ID NO: 22 - protein, 123 aa, Artificial Sequence
  4133 gH3 V-region - IGHV4-4 framework
EVQLQESGPGLVKPSGTLSLTCAASGFSFSGNYYMSWVRQPPGKGLEWIGSLYTGSSGSTYYASWAKGRVTISKDSSKTQVSLKLSSVTAADTAVYYCARDLGYEIDGYGGLWGQGTLVTVSS

SEQ ID NO: 23 - protein, 10 aa, Artificial Sequence
  CDRH1
GYTFTSYTMH

SEQ ID NO: 24 - protein, 17 aa, Artificial Sequence
  CDRH2
YINPSSGYTEYNQKFKD

SEQ ID NO: 25 - protein, 12 aa, Artificial Sequence
  CDRH3
VGDGFYPSWLAY

SEQ ID NO: 26 - protein, 12 aa, Artificial Sequence
  CDRH3 variant 1
VGDSFYPSWLAY

SEQ ID NO: 27 - protein, 12 aa, Artificial Sequence
  CDRH3 variant 2
VGDAFYPSWLAY

SEQ ID NO: 28 - protein, 12 aa, Artificial Sequence
  CDRH3 variant 3
VGDTFYPSWLAY

SEQ ID NO: 29 - protein, 11 aa, Artificial Sequence
  CDRL1
KASQSVRNDVA

SEQ ID NO: 30 - protein, 7 aa, Artificial Sequence
  CDRL2
YASKRYT

SEQ ID NO: 31 - protein, 9 aa, Artificial Sequence
  CDRL3
QQDYSSPTT

SEQ ID NO: 32 - protein, 107 aa, Artificial Sequence
  mouse antibody 6294 VL region
DIQMTQSPKFLLVSAGDRVTITCKASQSVRNDVAWYQQKPGQSPKLLIFYASKRYTGVPDRFTGSGYGTDFTFTISTVQAEDLAVYFCQQDYSSPTTFGGGTKLEIK

SEQ ID NO: 33 - protein, 121 aa, Artificial Sequence
  mouse antibody 6294 VH region
EVQLVESGAELARPGASVKMSCKASGYTFTSYTMHWVKQRPGQGLEWIGYINPSSGYTEYNQKFKDKTTLTADKSSSTAYLQLSSLTSEDSAVYYCARVGDGFYPSWLAYWGQGTLVTVSS

SEQ ID NO: 34 - protein, 121 aa, Artificial Sequence
  6294 gH2 V-region - IGHV3-48 framework
EVQLVESGGGLVQPGGSLRLSCAASGYTFTSYTMHWVRQAPGKGLEWIGYINPSSGYTEYNQKFKDRFTISADKAKSSLYLQMNSLRDEDTAVYYCARVGDGFYPSWLAYWGQGTLVTVSS

SEQ ID NO: 35 - protein, 121 aa, Artificial Sequence
  6294 gH4 V-region - IGHV1-69 framework
EVQLVQSGAEVKKPGSSVKVSCKASGYTFTSYTMHWVRQAPGQGLEWIGYINPSSGYTEYNQKFKDRVTITADKSTSTAYMELSSLRSEDTAVYYCARVGDGFYPSWLAYWGQGTLVTVSS

SEQ ID NO: 36 - protein, 107 aa, Artificial Sequence
  6294 gL2 V-region - IGKV1D-33 framework
DIQMTQSPSSLSASVGDRVTITCKASQSVRNDVAWYQQKPGKAPKLLIFYASKRYTGVPSRFTGSGYGTDFTFTISSLQPEDIAVYFCQQDYSSPTTFGGGTKVEIK

SEQ ID NO: 37 - protein, 107 aa, Artificial Sequence
  6294 gL3 V-region - IGKV4-1 framework
DIVMTQSPDSLAVSLGERATINCKASQSVRNDVAWYQQKPGQPPKLLIFYASKRYTGVPDRFTGSGYGTDFTLTISSLQAEDVAVYFCQQDYSSPTTFGGGTKVEIK

SEQ ID NO: 38 - protein, 483 aa, Artificial Sequence
  4133-6294 BYbe heavy chain
QEQLVESGGGLVQPEGSLTLTCTASGFSFSGNYYMCWVRQAPGKGLEWIGCLYTGSSGSTYYASWAKGRFTISKTSSTTVTLQMTSLTAADTATYFCARDLGYEIDGYGGLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPGGGGSGGGGSEVQLVESGAELARPGASVKMSCKASGYTFTSYTMHWVKQRPGQCLEWIGYINPSSGYTEYNQKFKDKTTLTADKSSSTAYLQLSSLTSEDSAVYYCARVGDGFYPSWLAYWGQGTLVTVSSGGGGSGGGGSGGGGSGGGGSDIQMTQSPKFLLVSAGDRVTITCKASQSVRNDVAWYQQKPGQSPKLLIFYASKRYTGVPDRFTGSGYGTDFTFTISTVQAEDLAVYFCQQDYSSPTTFGCGTKLEIK

SEQ ID NO: 39 - protein, 216 aa, Artificial Sequence
  4133-6294 BYbe light chain
AQVLTQTPSPVSAVVGGTVSISCQASQSVYNNNNLSWYQQKPGQPPKLLIYDASKLASGVPSRFKGSGSGTQFTLTISGVQCDDAATYYCLGGYYSSGWYFAFGGGTKVVVKRTPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 40 - protein, 369 aa, Artificial Sequence
  CD45 Domains 1-4 with TEV cleavage site and 10-Histidine tag
KPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSGTKENLYFQGHHHHHHHHHH

SEQ ID NO: 41 - protein, 1304 aa, Artificial Sequence
  Human CD45
MYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 42 - protein, 8 aa, Artificial Sequence
  Hinge linker sequence
DKTHTCAA

SEQ ID NO: 43 - protein, 11 aa, Artificial Sequence
  Hinge linker sequence
DKTHTCPPCPA

SEQ ID NO: 44 - protein, 18 aa, Artificial Sequence
  Hinge linker sequence
DKTHTCPPCPATCPPCPA

SEQ ID NO: 45 - protein, 25 aa, Artificial Sequence
  Hinge linker sequence
DKTHTCPPCPATCPPCPATCPPCPA

SEQ ID NO: 46 - protein, 30 aa, Artificial Sequence
  Hinge linker sequence
DKTHTCPPCPAGKPTLYNSLVMSDTAGTCY

SEQ ID NO: 47 - protein, 31 aa, Artificial Sequence
  Hinge linker sequence
DKTHTCPPCPAGKPTHVNVSVVMAEVDGTCY

SEQ ID NO: 48 - protein, 15 aa, Artificial Sequence
  Hinge linker sequence
DKTHTCCVECPPCPA

SEQ ID NO: 49 - protein, 26 aa, Artificial Sequence
  Hinge linker sequence
DKTHTCPRCPEPKSCDTPPPCPRCPA

SEQ ID NO: 50 - protein, 11 aa, Artificial Sequence
  Hinge linker sequence
DKTHTCPSCPA

SEQ ID NO: 51 - protein, 7 aa, Artificial Sequence
  Flexible linker sequence
SGGGGSE

SEQ ID NO: 52 - protein, 6 aa, Artificial Sequence
  Flexible linker sequence
DKTHTS

SEQ ID NO: 53 - protein, 6 aa, Artificial Sequence
  Flexible linker sequence
SGGGGS

SEQ ID NO: 54 - protein, 11 aa, Artificial Sequence
  Flexible linker sequence
SGGGGSGGGGS

SEQ ID NO: 55 - protein, 16 aa, Artificial Sequence
  Flexible linker sequence
SGGGGSGGGGSGGGGS

SEQ ID NO: 56 - protein, 21 aa, Artificial Sequence
  Flexible linker sequence
SGGGGSGGGGSGGGGSGGGGS

SEQ ID NO: 57 - protein, 26 aa, Artificial Sequence
  Flexible linker sequence
SGGGGSGGGGSGGGGSGGGGSGGGGS

SEQ ID NO: 58 - protein, 11 aa, Artificial Sequence
  Flexible linker sequence
AAAGSGGASAS

SEQ ID NO: 59 - protein, 16 aa, Artificial Sequence
  Flexible linker sequence
  misc_feature (7)..(7): Xaa can be any naturally occurring amino acid
AAAGSGXGGGSGASAS

SEQ ID NO: 60 - protein, 21 aa, Artificial Sequence
  Flexible linker sequence
  misc_feature (7)..(7): Xaa can be any naturally occurring amino acid
  misc_feature (12)..(12): Xaa can be any naturally occurring amino acid
AAAGSGXGGGSXGGGSGASAS

SEQ ID NO: 61 - protein, 26 aa, Artificial Sequence
  Flexible linker sequence
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
AAAGSGXGGGSXGGGSXGGGSGASAS

SEQ ID NO: 62 - protein, 31 aa, Artificial Sequence
  Flexible linker sequence
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
  misc_feature (22)..(22): Xaa can be any naturally occurring amino acid
AAAGSGXGGGSXGGGSXGGGSXGGGSGASAS

SEQ ID NO: 63 - protein, 13 aa, Artificial Sequence
  Flexible linker sequence
  misc_feature (7)..(7): Xaa can be any naturally occurring amino acid
AAAGSGXSGASAS

SEQ ID NO: 64 - protein, 28 aa, Artificial Sequence
  Flexible linker sequence
PGGNRGTTTTRRPATTTGSSPGPTQSHY

SEQ ID NO: 65 - protein, 11 aa, Artificial Sequence
  Flexible linker sequence
ATTTGSSPGPT

SEQ ID NO: 66 - protein, 6 aa, Artificial Sequence
  Flexible linker sequence
ATTTGS

SEQ ID NO: 67 - protein, 11 aa, Artificial Sequence
  Linker
SGGGGSGGGGS

SEQ ID NO: 68 - protein, 21 aa, Artificial Sequence
  Flexible linker sequence
EPSGPISTINSPPSKESHKSP

SEQ ID NO: 69 - protein, 15 aa, Artificial Sequence
  Flexible linker sequence
GTVAAPSVFIFPPSD

SEQ ID NO: 70 - protein, 15 aa, Artificial Sequence
  Flexible linker sequence
GGGGIAPSMVGGGGS

SEQ ID NO: 71 - protein, 15 aa, Artificial Sequence
  Flexible linker sequence
GGGGKVEGAGGGGGS

SEQ ID NO: 72 - protein, 15 aa, Artificial Sequence
  Flexible linker sequence
GGGGSMKSHDGGGGS

SEQ ID NO: 73 - protein, 15 aa, Artificial Sequence
  Flexible linker sequence
GGGGNLITIVGGGGS

SEQ ID NO: 74 - protein, 15 aa, Artificial Sequence
  Flexible linker sequence
GGGGVVPSLPGGGGS

SEQ ID NO: 75 - protein, 12 aa, Artificial Sequence
  Flexible linker sequence
GGEKSIPGGGGS

SEQ ID NO: 76 - protein, 18 aa, Artificial Sequence
  Flexible linker sequence
RPLSYRPPFPFGFPSVRP

SEQ ID NO: 77 - protein, 18 aa, Artificial Sequence
  Flexible linker sequence
YPRSIYIRRRHPSPSLTT

SEQ ID NO: 78 - protein, 18 aa, Artificial Sequence
  Flexible linker sequence
TPSHLSHILPSFGLPTFN

SEQ ID NO: 79 - protein, 18 aa, Artificial Sequence
  Flexible linker sequence
RPVSPFTFPRLSNSWLPA

SEQ ID NO: 80 - protein, 18 aa, Artificial Sequence
  Flexible linker sequence
SPAAHFPRSIPRPGPIRT

SEQ ID NO: 81 - protein, 18 aa, Artificial Sequence
  Flexible linker sequence
APGPSAPSHRSLPSRAFG

SEQ ID NO: 82 - protein, 18 aa, Artificial Sequence
  Flexible linker sequence
PRNSIHFLHPLLVAPLGA

SEQ ID NO: 83 - protein, 18 aa, Artificial Sequence
  Flexible linker sequence
MPSLSGVLQVRYLSPPDL

SEQ ID NO: 84 - protein, 18 aa, Artificial Sequence
  Flexible linker sequence
SPQYPSPLTLTLPPHPSL

SEQ ID NO: 85 - protein, 18 aa, Artificial Sequence
  Flexible linker sequence
NPSLNPPSYLHRAPSRIS

SEQ ID NO: 86 - protein, 17 aa, Artificial Sequence
  Flexible linker sequence
LPWRTSLLPSLPLRRRP

SEQ ID NO: 87 - protein, 18 aa, Artificial Sequence
  Flexible linker sequence
PPLFAKGPVGLLSRSFPP

SEQ ID NO: 88 - protein, 18 aa, Artificial Sequence
  Flexible linker sequence
VPPAPVVSLRSAHARPPY

SEQ ID NO: 89 - protein, 17 aa, Artificial Sequence
  Flexible linker sequence
LRPTPPRVRSYTCCPTP

SEQ ID NO: 90 - protein, 18 aa, Artificial Sequence
  Flexible linker sequence
PNVAHVLPLLTVPWDNLR

SEQ ID NO: 91 - protein, 18 aa, Artificial Sequence
  Flexible linker sequence
CNPLLPLCARSPAVRTFP

SEQ ID NO: 92 - protein, 11 aa, Artificial Sequence
  Rigid Linker
GAPAPAAPAPA

SEQ ID NO: 93 - protein, 4 aa, Artificial Sequence
  Rigid linker
PPPP

SEQ ID NO: 94 - protein, 11 aa, Artificial Sequence
  Hinge linker sequence
DLCLRDWGCLW

SEQ ID NO: 95 - protein, 11 aa, Artificial Sequence
  Hinge linker sequence
DICLPRWGCLW

SEQ ID NO: 96 - protein, 15 aa, Artificial Sequence
  Hinge linker sequence
MEDICLPRWGCLWGD

SEQ ID NO: 97 - protein, 20 aa, Artificial Sequence
  Hinge linker sequence
QRLMEDICLPRWGCLWEDDE

SEQ ID NO: 98 - protein, 20 aa, Artificial Sequence
  Hinge linker sequence
QGLIGDICLPRWGCLWGRSV

SEQ ID NO: 99 - protein, 21 aa, Artificial Sequence
  Hinge linker sequence
QGLIGDICLPRWGCLWGRSVK

SEQ ID NO: 100 - protein, 15 aa, Artificial Sequence
  Hinge linker sequence
EDICLPRWGCLWEDD

SEQ ID NO: 101 - protein, 18 aa, Artificial Sequence
  Hinge linker sequence
RLMEDICLPRWGCLWEDD

SEQ ID NO: 102 - protein, 16 aa, Artificial Sequence
  Hinge linker sequence
MEDICLPRWGCLWEDD

SEQ ID NO: 103 - protein, 15 aa, Artificial Sequence
  Hinge linker sequence
MEDICLPRWGCLWED

SEQ ID NO: 104 - protein, 18 aa, Artificial Sequence
  Hinge linker sequence
RLMEDICLARWGCLWEDD

SEQ ID NO: 105 - protein, 20 aa, Artificial Sequence
  Hinge linker sequence
EVRSFCTRWPAEKSCKPLRG

SEQ ID NO: 106 - protein, 20 aa, Artificial Sequence
  Hinge linker sequence
RAPESFVCYWETICFERSEQ

SEQ ID NO: 107 - protein, 11 aa, Artificial Sequence
  Hinge linker sequence
EMCYFPGICWM

SEQ ID NO: 108 - protein, 6 aa, Artificial Sequence
  Linker
ASGGGG

SEQ ID NO: 109 - protein, 8 aa, Artificial Sequence
  Linker
ASGGGGSG

SEQ ID NO: 110 - DNA, 132 bp, Artificial Sequence
  GCN4 sequence
gctagcggaggcggaagaatgaaacaacttgaacccaaggttgaagaattgcttccgaaaaattatcacttggaaaatgaggttgccagattaaagaaattagttggcgaacgccatcaccatcaccatcac

SEQ ID NO: 111 - protein, 44 aa, Artificial Sequence
  GCN4 sequence
ASGGGRMKQLEPKVEELLPKNYHLENEVARLKKLVGERHHHHHH

SEQ ID NO: 112 - DNA, 792 bp, Artificial Sequence
  52SR4 sequence
gatgcggtggtgacccaggaaagcgcgctgaccagcagcccgggcgaaaccgtgaccctgacctgccgcagcagcaccggcgcggtgaccaccagcaactatgcgagctgggtgcaggaaaaaccggatcatctgtttaccggcctgattggcggcaccaacaaccgcgcgccgggcgtgccggcgcgctttagcggcagcctgattggcgataaagcggcgctgaccattaccggcgcgcagaccgaagatgaagcgatttatttttgcgtgctgtggtatagcgaccattgggtgtttggctgcggcaccaaactgaccgtgctgggtggaggcggtggctcaggcggaggtggctcaggcggtggcgggtctggcggcggcggcagcgatgtgcagctgcagcagagcggcccgggcctggtggcgccgagccagagcctgagcattacctgcaccgtgagcggctttctcctgaccgattatggcgtgaactgggtgcgccagagcccgggcaaatgcctggaatggctgggcgtgatttggggcgatggcattaccgattataacagcgcgctgaaaagccgcctgagcgtgaccaaagataacagcaaaagccaggtgtttctgaaaatgaacagcctgcagagcggcgatagcgcgcgctattattgcgtgaccggcctgtttgattattggggccagggcaccaccctgaccgtgagcagcgcggccgcccatcaccatcaccatcacgaacagaaactgattagcgaagaagatctgtaatag

SEQ ID NO: 113 - protein, 262 aa, Artificial Sequence
  52SR4 sequence
DAVVTQESALTSSPGETVTLTCRSSTGAVTTSNYASWVQEKPDHLFTGLIGGTNNRAPGVPARFSGSLIGDKAALTITGAQTEDEAIYFCVLWYSDHWVFGCGTKLTVLGGGGGSGGGGSGGGGSGGGGSDVQLQQSGPGLVAPSQSLSITCTVSGFLLTDYGVNWVRQSPGKCLEWLGVIWGDGITDYNSALKSRLSVTKDNSKSQVFLKMNSLQSGDSARYYCVTGLFDYWGQGTTLTVSSAAAHHHHHHEQKLISEEDL

SEQ ID NO: 114 - protein, 11 aa, Artificial Sequence
  Linker
SGGGGTGGGGS

SEQ ID NO: 115 - protein, 219 aa, Artificial Sequence
  4133 Light chain
AQVLTQTPSPVSAVVGGTVSISCQASQSVYNNNNLSWYQQKPGQPPKLLIYDASKLASGVPSRFKGSGSGTQFTLTISGVQCDDAATYYCLGGYYSSGWYFAFGGGTKVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 449 aa, Artificial Sequence
  4133 IgG4P FALA
QEQLVESGGGLVQPEGSLTLTCTASGFSFSGNYYMCWVRQAPGKGLEWIGCLYTGSSGSTYYASWAKGRFTISKTSSTTVTLQMTSLTAADTATYFCARDLGYEIDGYGGLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 451 aa, Artificial Sequence
  4133 IgG4P FALA Knob
QEQLVESGGGLVQPEGSLTLTCTASGFSFSGNYYMCWVRQAPGKGLEWIGCLYTGSSGSTYYASWAKGRFTISKTSSTTVTLQMTSLTAADTATYFCARDLGYEIDGYGGLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPSAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKTGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 214 aa, Artificial Sequence
  6294 Light chain
DIQMTQSPKFLLVSAGDRVTITCKASQSVRNDVAWYQQKPGQSPKLLIFYASKRYTGVPDRFTGSGYGTDFTFTISTVQAEDLAVYFCQQDYSSPTTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 448 aa, Artificial Sequence
  6294 IgG4P FALA Hole
EVQLVESGAELARPGASVKMSCKASGYTFTSYTMHWVKQRPGQGLEWIGYINPSSGYTEYNQKFKDKTTLTADKSSSTAYLQLSSLTSEDSAVYYCARVGDGFYPSWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

(19) United States
(12) Patent Application Publication
(10) Pub. No.: US 2023/0374148 A1
(43) Pub. Date: Nov. 23, 2023

(54) BINDING MOLECULES THAT MULTIMERISE CD45

(71) Applicant: UCB BIOPHARMA SRL, Brussels (BE)

(72) Inventors: Stephen Edward RAPECKI, Slough, Berkshire (GB); Ralph ADAMS, Slough, Berkshire (GB); David Paul HUMPHREYS, Slough, Berkshire (GB); Helen Margaret FINNEY, Slough, Berkshire (GB); Rosemary Frances BITHELL, Slough, Berkshire (GB)

(21) Appl. No.: 18/248,651

(22) PCT Filed: Oct. 14, 2021
(86) PCT No.: PCT/EP2021/078516

§ 371 Date: Apr. 11, 2023

(30) Foreign Application Priority Data
Oct. 15, 2020 (GB) 2016386.1
Jan. 20, 2021 (GB) 2100737.2

(51) Int. Cl. C07K 16/28; C07K 16/18; A61P 35/00; A61P 35/02

(52) U.S. Cl. CPC C07K 16/289; C07K 16/18; A61P 35/00; A61P 35/02; C07K 2317/31; C07K 2317/52; C07K 2317/24; C07K 2317/73; C07K 2317/72; C07K 2317/55; C07K 2317/92; C07K 2317/35

(57) ABSTRACT

The present invention provides binding molecule or molecules that are able to multimerise CD45 to induce cell death of a cell expressing CD45 without also inducing significant cytokine release. For example, the invention provides antibodies against CD45, wherein the antibodies comprise at least two different paratopes each specific for a different epitope of CD45. The antibodies may be used to cross-link CD45 on the surface of cells. The antibodies may be used in a variety of therapeutic ways including to deplete cells, for example prior to cell transplantation.

### FIELD OF INVENTION

The present invention relates to binding molecules, particularly antibodies, which are specific for CD45. The binding molecules may be, for example, used to kill target cells, particularly prior to the transplant of cells.

### BACKGROUND OF INVENTION

CD45, the first and prototypic receptor-like protein tyrosine phosphatase, is expressed on nucleated hematopoietic cells and plays a central role in the regulation of cellular responses. CD45 has also been known as PTPRC, T200, Ly5, leucocyte common antigen (LCA), and B220. CD45 is the most abundant cell surface protein in T and B cells. It is essential for B and T cell development and activation. Studies of CD45 mutant cell lines, CD45-deficient mice, and CD45-deficient humans initially demonstrated the essential role of CD45 in T and B cell antigen receptor signal transduction and lymphocyte development. It is now known that CD45 also modulates signals emanating from integrin and cytokine receptors. In contrast to its positive role in antigen receptor signalling, CD45 acts as a negative-regulator of integrin mediated signalling for instance in macrophages. CD45 may also play a role in regulating haematopoiesis and interferon-dependent antiviral responses. CD45 can also play a role in cell survival.

CD45 comprises a highly and variably glycosylated extracellular domain of approximately 400 to 550 amino acids, followed by a single transmembrane domain and a long intracellular domain of 705 amino acids, containing two tandemly repeated phosphatase domains. The regulation of CD45 expression and the expression of multiple alternative splicing isoforms (which alternatively splice exons 4,5 and 6 from the CD45 gene and are designated A, B and C) critically regulates phosphatase activity and differential signal transduction. CD45 affects cellular responses by controlling the relative threshold of sensitivity to external stimuli. Perturbation of this function may contribute to autoimmunity, immunodeficiency, and malignancy.

All CD45 isoforms display tyrosine phosphatase activity which is mediated by the cytoplasmic domain of the molecule comprising the two tandem repeats of phosphatase domains D1 and D2, with each containing a highly conserved HC(X)₅R motif. All of the tyrosine phosphatase activity of CD45 is thought to arise from the D1 domain, with the D2 domain possibly involved in regulation. One of the primary targets for CD45 tyrosine phosphatase are Src-family kinases, reflecting the role of CD45 in cell signalling. Depending on where the phosphatase activity of CD45 acts it may activate or down-regulate the activity of such Src-family kinases.

Given the importance of CD45, there is an ongoing need for agents that can target and modulate CD45.

### SUMMARY OF THE INVENTION

The present invention provides, amongst other things, binding molecules able to multimerise CD45 on a target cell to induce cell death, whilst not inducing significant cytokine release. Without wishing to be bound by this theory, it is thought that the binding molecules of the present invention are better able to cross-link CD45 molecules than known binding molecules and so have an improved ability to induce cell death in the target cell. The binding molecules of the present invention may be therefore used to kill target cells, particularly prior to cell transplantation in a subject. In some embodiments, a binding molecule is provided. In other embodiments a mixture of at least two different binding molecules is provided.

As discussed in detail herein, the binding molecules of the present invention are provided in a variety of formats. In one particularly preferred embodiment, a binding molecule of the invention is an antibody. In a further particularly preferred embodiment, a mixture of at least two different binding molecules of the present invention is a mixture of at least two different antibodies. Antibody formats which may be employed in the various embodiments of the present invention are discussed in detail herein. In one preferred embodiment, the antibody is an IgG antibody. In one embodiment the IgG antibody is an IgG1, IgG2, or IgG4 antibody. In an especially preferred embodiment, the antibody is an IgG4 antibody.

Examples of preferred IgG formats include: IgG with altered hinges (for example altered length and/or disulphide bonds); IgG with altered glycans; IgG with altered FcRn binding (for example with such altered binding in order to reduce serum half-life); IgG with heavy chain modifications favouring heterodimer formation over homodimer formation (for example knobs-in-holes and/or charge modifications); IgG with heavy chain modifications altering binding to a purification agent (in particular where one heavy chain has a modification changing binding to Protein A and the other does not, as a way to favour purification of heterodimers over homodimers); IgG with altered effector functions (for example altered FcGR binding and/or C1q binding); and/or IgG with reduced/no effector functions. In one particularly preferred embodiment, such formats are employed for an IgG antibody. In another particularly preferred embodiment, an antibody employed in the invention is an IgG4 antibody with knob-in-hole modifications. In a further preferred embodiment, the antibody is an IgG4 antibody with knob-in-hole modifications and FALA modification. In one particularly preferred embodiment, such IgG formats will be employed where the antibody has two different specificities for CD45.

The invention is not though limited to IgG format antibodies and any appropriate binding molecule, in particular those described herein, may be employed. For example, non-IgG antibodies may be employed. TrYbe and BYbe format antibodies, particularly those described herein, may be employed. Also non-antibody binding molecules may be employed as also described herein.

Accordingly, the present invention provides:
An antibody comprising at least two different paratopes, each being specific for a different epitope of CD45.
A nucleic acid molecule or molecules encoding an antibody of the invention.
A vector or vectors encoding an antibody of the invention or comprising a nucleic acid molecule or molecules of the invention.
A pharmaceutical composition comprising: (a) an antibody of the invention, a nucleic acid molecule or molecules of the invention, or a vector or vectors of the invention; and (b) a pharmaceutically acceptable carrier or diluent.
A binding molecule or molecules that are able to multimerise CD45 to induce cell death of a cell expressing CD45 without also inducing significant cytokine release.
A nucleic acid molecule or molecules encoding a binding molecule or molecules of the invention.
A vector or vectors encoding a binding molecule or molecules of the invention or comprising a nucleic acid molecule or molecules of the invention.
A pharmaceutical composition comprising: (a) a binding molecule or molecules of the invention, a nucleic acid molecule or molecules of the invention, or a vector or vectors of the invention; and (b) a pharmaceutically acceptable carrier or diluent.
A pharmaceutical composition of the invention for use in a method of therapy.
A pharmaceutical composition of the invention for use in a method of killing disease-associated CD45-expressing cells in a subject.
A method of killing disease-associated, CD45-expressing cells in a subject, the method comprising administering a pharmaceutical composition of the invention to the subject.
Use of a binding molecule or molecules of the invention, a nucleic acid molecule or molecules of the invention, or a vector or vectors of the invention in the manufacture of a medicament for killing disease-associated, CD45-expressing cells in a subject.
A method of screening for a binding molecule or molecules able to multimerise CD45 to induce cell death, the method comprising: (a) contacting a binding molecule or molecules that are able to bind CD45 with target cells expressing CD45; and (b) determining whether the target cells have undergone cell death.
An ex vivo method of depleting or killing target cells expressing CD45 in a population of cells, tissue, or organ comprising contacting said cells tissue or organ with a binding molecule of the invention or an antibody of the invention.
A binding molecule of the invention or an antibody of the invention for use in a method of treating or preventing graft versus host disease (GVHD) in a subject, the method comprising: (a) contacting ex vivo a population of cells, tissue, or organ with a binding molecule of the invention or an antibody of the invention to kill target cells expressing CD45; and (b) transplanting the treated population of cells, tissue, or organ to said subject.
A method of treating or preventing graft versus host disease (GVHD) comprising: (a) contacting a population of cells, tissue, or organ with a binding molecule of the present invention or an antibody of the present invention to kill target cells expressing CD45 ex vivo; and (b) transplanting the treated population of cells, tissue, or organ to a subject in need of such a transplantation.
Use of a binding molecule of the present invention or an antibody of the present invention in the manufacture of a medicament for treating or preventing graft versus host disease (GVHD) in a method comprising: (a) contacting a population of cells, tissue, or organ with a binding molecule of the present invention or an antibody of the present invention to kill target cells expressing CD45 ex vivo; and (b) transplanting the treated population of cells, tissue, or organ to a subject in need of such a transplantation.

### BRIEF DESCRIPTION OF THE FIGURES

FIG. 1 Bar charts showing (A) lymphocyte cell count and (B) percentage of lymphocytes which are apoptotic, following incubation with combinations of Fab-X and Fab-Y with either specificity for CD45 or an irrelevant antigen. Apoptosis is measured by Annexin V binding.

FIG. 2 Graph showing the titration of the effect on CD4+ T cells by combinations of Fab-X and Fab-Y with either specificity for CD45 or an irrelevant antigen. Values are percentage reduction of T cell counts relative to untreated cells.

FIG. 3 Graphs showing the titration of the effect on subsets of cells in PBMCs by either (A) a combination of Fab-X and Fab-Y with specificity for CD45, 6294-X/4133-Y, or (B) a BYbe (Fab-scFv) with specificity for CD45, 4133-6294 BYbe. Values are percentage reduction of subset cell counts relative to untreated cells.

FIG. 4 Graphs showing the titration of the effect of on (A) lymphocyte cells and (B) CD⁴⁺ cells in whole blood from donor HTA #051119-01, and on (C) lymphocyte cells and (D) CD⁴⁺ cells in whole blood from donor HTA #051119-02, by either a combination of Fab-X and Fab-Y with specificity for CD45 (6294-X/4133-Y), an anti-CD45 BYbe (4133-6294 BYbe) or a BYbe with irrelevant specificity (NegCtrl BYbe). Values are percentage reduction of cell counts relative to untreated cells.

FIG. 5 Bar charts showing levels of induction of (A) CCL2, (B) GM-CSF, (C) IL-RA, (D) IL-6, (E) IL-8, (F) IL-10, (G) IL-11 or (H) M-CSF in whole blood by either an anti-CD45 BYbe (4133-6294 BYbe), a BYbe with irrelevant specificity (NegCtrl BYbe), Campath or PBS.

FIG. 6 Bar chart showing the effect on levels of T cells in whole blood by either a combination of Fab-X and Fab-Y with specificity for CD45 (6294-X/4133-Y), an anti-CD45 BYbe (4133-6294 BYbe), a BYbe with irrelevant specificity (NegCtrl BYbe), Campath or PBS.

FIG. 7 Graphs showing levels of induction of (A) IFNγ, (B) IL-6 and (C) TNFα in whole blood by either a combination of Fab-X and Fab-Y with specificity for CD45 (6294-X/4133-Y), an anti-CD45 BYbe (4133-6294 BYbe), a BYbe with irrelevant specificity (NegCtrl BYbe), Campath or PBS.

FIG. 8 Images taken with IncuCyte® S3 system showing (A) M1 macrophages and (B) M2 macrophages.

FIG. 9 Bar charts showing the effect on viability of (A) M1 macrophages and (B) M2 macrophages by either an anti-CD45 BYbe (4133-6294 BYbe), a BYbe with irrelevant specificity (NegCtrl BYbe), Camptothecin, Staurosporine or PBS. Values are Raw Luminescent Units (RLU).

FIG. 10 Graphs showing the levels of induction of Caspase 3/7 in (A) M1 macrophages and (B) M2 macrophages by an anti-CD45 BYbe (4133-6294 BYbe), a BYbe with irrelevant specificity (NegCtrl BYbe), Camptothecin, or PBS.

FIG. 11 Graphs showing mass photometry signals of (A) CD45 ECD (B) 4133-6294 BYbe and (C) a mixture of CD45 ECD and 4133-6294 BYbe. Values are counts detected versus mass (kDa). The schematic representation of CD45 ECD was generated from PDB code SFMV. The schematic representation of 4133-6294 BYbe is a model generated by linking in-house crystal structures of a Fab and a scFv. The schematic representation of a 4133-6294 BYbe-CD45 ECD complex and the higher order multimeric forms are models. The models are for illustrative purposes only and are not intended to indicate the specific location of epitopes.

FIG. 12 Sequences of the V-regions of antibodies 4133 and 6294, humanised grafts of antibodies 4133 and 6294, 4133-6294 BYbe heavy and light chains, and CD45 domains 1-4 of extracellular domain. The predicted N-linked glycosylation sites in the CD45 ECD are underlined. Also shown are sequences of 4133 and 6294 chimeric light and heavy IgG4P FALA chains.

FIG. 13 Graphs showing the titration of the effect of either anti-CD45 6294-X/4133-Y ((A) & (C)) or anti-CD45 4133-6294 BYbe ((B) & (D)) on lymphocyte cells and CD34⁺ cells in PBMCs. Values are shown as percentage reduction of cell counts relative to untreated cells in (A) & (B) with the actual cell counts shown in (C) & (D).

FIG. 14 Graph showing the sedimentation velocity, as measured in an analytical ultracentrifuge, of a molar 1:1 mixture of CD45 ECD and 4133-6294 BYbe (solid black line). Overlaid onto the graph are the sedimentation velocities of CD45 ECD (dots) and 4133-6294 BYbe (dashes). Values are continuous distribution (fringes/S) versus sedimentation coefficient (10⁻¹³ seconds). The schematic representation of CD45 ECD was generated from PDB code SFMV. The schematic representation of 4133-6294 BYbe is a model generated by linking in-house crystal structures of a Fab and a scFv. The schematic representation of a 4133-6294 BYbe-CD45 ECD complex and the higher order multimeric forms are models. The models are for illustrative purposes only and are not intended to indicate the specific location of epitopes.

FIG. 15 Graph showing the titration of the effect of either anti-CD45 4133-6294 IgG4P FALA KiH, anti-CD45 4133-6294 BYbe or anti-CD45 4133 IgG4P FALA on lymphocyte cells in PBMCs. Values are shown as percentage reduction of cell counts relative to untreated cells.

FIG. 16 Graph showing the titration of the effect of either anti-CD45 4133 IgG4P FALA, anti-CD45 4133-6294 BYbe or a combination of anti-CD45 4133 IgG4P FALA and anti-CD45 6294-X/6294-Y on lymphocyte cells in PBMCs. Values are shown as percentage reduction of cell counts relative to untreated cells.

FIG. 17 Graphs showing the titration of the effect of either an anti-CD45 4133-6294 BYbe, an anti-CD45 4133-6294-645 TrYbe or an anti-CD45 4133-6294 IgG4 FALA KiH on lymphocyte cells in PBMC. Values are shown as percentage reduction of cell counts.

FIG. 18 Graphs showing the titration of the effect on cell lines (A) Jurkat (B) CCRF-SB (C) MC116 (D) Raji and Ramos (E) SU-DHL-4, SU-DHL-5, SU-DHL-8, NU-DUL-1 and OCI-Ly3 (F) THP-1 (G) Dakiki, by an anti-CD45 BYbe (4133-6294 BYbe). Values are percentage reduction of cell counts.

FIG. 19 Bar charts showing percentage reduction of cell counts of cell lines (A) Jurkat (B) CCRF-SB (C) MC116 (D) Raji (E) Ramos (F) SU-DHL-4 (G) SU-DHL-5 (H) SU-DHL-8 (I) NU-DUL-1 (J) OCI-Ly3 (K) THP-1 (L) Dakiki, by either NegCtrl BYbe (a BYbe with irrelevant specificity, only top concentration, 500 nM, of dilution series is shown), Staurosporin, Camptothecin, Rituximab, Campath or Anti-Thymocyte Globulin (ATG). The Top % cell reduction by 4133-6294 BYbe is also marked.

### DETAILED DESCRIPTION OF THE INVENTION

The present invention provides, amongst other things, binding molecules that are able to multimerise CD45 to induce cell death of a target cell without significantly inducing cytokine release. In an especially preferred embodiment, the binding molecules are antibodies. More details of the binding molecules and their uses are provided below.

### CD45 Molecules

The binding molecules of the present invention are specific for CD45. As explained above, CD45 is a member of the protein tyrosine phosphatase (PTP) family. PTPs are known to be signalling molecules that regulate a variety of cellular processes including cell growth, differentiation, mitotic cycle, and oncogenic transformation. CD45 contains an extracellular domain, a single transmembrane segment and two tandem intracytoplasmic catalytic domains, and thus belongs to receptor type PTP. Various isoforms of CD45 exist: CD45RA, CD45RB, CD45RC, CD45RAB, CD45RAC, CD45RBC, CD45RO, CD45R (ABC). CD45 splice variant isoforms A, B and C are expressed differentially on many leucocyte subsets. Despite the existence of different isoforms of CD45, they share common sequences that mean all of the isoforms can be targeted by one binding molecule, and in particular by one antibody.

The intracellular (COOH-terminal) region of CD45 contains two PTP catalytic domains, and the extracellular region is highly variable due to alternative splicing of exons 4, 5, and 6 (designated A, B, and C, respectively), plus differing levels of glycosylation. The CD45 isoforms detected are cell type, maturation, and activation state-specific. In general, the long form of the protein (A, B or C) is expressed on naïve or unactivated B cells and the mature or truncated form of CD45 (RO) is expressed on activated or mature/memory B cells.

The human sequence for CD45 is available in UniProt entry number P08575 and provided herein in SEQ ID NO: 41, or amino acids 24-1304 of SEQ ID NO: 41, lacking the signal peptide. The amino acid sequence of human CD45 domains 1-4 of the extracellular domain is provided in SEQ ID NO: 113. The murine version of CD45 is provided in UniProt entry P06800. The present invention relates to all forms of CD45, from any species. In one embodiment, the CD45 is a mammalian CD45. In one particularly preferred embodiment CD45 refers to the human form of the protein and natural variants and isoforms thereof. In one preferred embodiment, a binding molecule of the present invention, particularly an antibody of the present invention, is able to bind all isoforms of CD45 expressed by a given species. For example, a binding molecule, in particular an antibody, may bind all human isoforms of CD45. In one embodiment where a mixture of binding molecules, in particular a mixture of antibodies is employed, collectively they may be able to bind to all of the isoforms of CD45 for a species and in particular all human isoforms of CD45. In an alternative embodiment, a binding molecule of the present invention, particularly an antibody of the present invention, is specific for a particular isoform of CD45. In another embodiment, a binding molecule of the present invention is able to bind rodent CD45, for example it is able to bind both rodent and human CD45.

In one preferred embodiment, a binding molecule of the present invention, particularly an antibody of the present invention, is able to bind all of the isoforms of CD45 expressed by a subject. In another preferred embodiment, a binding molecule of the present invention, in particular an antibody of the present invention, is able to specifically bind all of the isoforms of CD45 expressed by a subject, but not other proteins. In another preferred embodiment, a binding molecule of the present invention, particularly an antibody of the present invention, recognises the extracellular region of CD45 common to all of the isoforms of CD45 expressed by a subject. In one preferred embodiment, a binding molecule of the present invention, particularly an antibody of the present invention, comprises at least two different specificities each specifically binding to a different epitope within the extracellular domains of CD45 whose sequence is provided as SEQ ID No:113. In an alternative embodiment, a binding molecule or molecules, in particular an antibody or antibodies, of the present invention binds an intracellular region of CD45.

### Binding Molecules

The present invention provides binding molecules and in particular binding molecules that are specific for CD45. In an especially preferred embodiment, a binding molecule of the present invention is an antibody. Alternatively, a binding molecule of the present invention is not an antibody. What is set out herein for antibodies may be also applied to binding molecules of the present invention in general and vice versa unless specifically stated otherwise. In one embodiment, a binding molecule of the present invention that is not an antibody may comprise a biocompatible framework structure used in a binding domain of the molecule having a structure based on protein scaffolds or skeletons other than immunoglobulin domains. Examples of alternative binding molecules of the present invention include those based on fibronectin, ankyrin, lipocalin, neocarzinostain, cytochrome b, CP1 zinc finger, PST1, coiled coil, LACI-D1, Z domain and tendramisat domains (see for example, Nygren and Uhlen, 1997, Current Opinion in Structural Biology, 7, 463-469). The term ‘binding molecules’ as used herein also includes binding molecules based on biological scaffolds including Adnectins, Affibodies, Darpins, Phylomers, Avimers, Aptamers, Anticalins, Tetranectins, Microbodies, Affilins and Kunitz domains.

Small molecules able to bind CD45 may be also used as binding molecules of the present invention. In one embodiment the small molecules that may be employed include, for instance, peptides, cyclised peptides and macrocycles. For example, peptide-mRNA libraries may be used to identify desired peptides. In one embodiment, libraries of such molecules are converted to cDNA-peptide, then screened to identify peptides with the necessary ability to bind CD45, and then the selected cDNA peptide with the desired property subjected to PCR to identify the sequence of the cDNA and hence peptide. In one embodiment, the Extreme Diversity™ platform of Ra Pharma may be employed for such screening. In another embodiment, libraries of peptides modified with a scaffold may be screened for their ability to bind CD45, for example using the approach of Bicycle Therapeutics for such library screening.

A binding molecule of the present invention will have at least one specificity for CD45. The “specificity” of a binding molecule denotes the target a binding molecule binds and typically in the context of the present invention also denotes where on the target the binding molecule binds. So, for instance, two specificities of a binding molecule may be both specific for CD45, but bind different portions of CD45 itself, and so represent different specificities for CD45. Typically, a particular portion or portions of the binding molecule will bind CD45, for instance a binding site of the binding molecule will bind CD45. In the case of an antibody, an antigen-binding site will bind CD45 and confer the specificity. In one embodiment, the portion of an antibody that binds CD45 is referred to as a paratope of the antibody specific for CD45. The bound portion of CD45 may, for example, be referred to as the epitope of the antibody.

In one embodiment, a binding molecule of the present invention shows trans binding, that is it binds more than one molecule of CD45 at the same time. Such trans binding typically results in cross-linking of CD45 and hence represents an especially preferred embodiment of the present invention. In one embodiment, a binding molecule of the invention may display cis binding of CD45 so that it binds just one molecule of CD45 with its binding sites. In such embodiments, a further binding agent may be used to cross-link the binding molecules bound to different molecules of CD45.

Binding molecules of the present invention, and in particular antibodies of the present invention, may be therefore multi-specific in the sense that they may comprise at least two different specificities that each bind a different portion, particularly a different epitope, of CD45. A multi-specific or bispecific binding molecule in the context of the present invention does not therefore necessarily require binding to different molecules: it encompasses the binding molecule of the present invention, particularly the antibody of the present invention, comprising different binding sites that bind different sites on the same target molecule and especially on CD45. As discussed further below, binding molecules of the present invention may comprise further specificities for targets other than CD45, as well as for CD45. In one further embodiment, the further specificity is for serum albumin.

In a preferred embodiment, a binding molecule of the present invention may comprise two different specificities for CD45. In one preferred embodiment, the two different specificities bind portions of CD45 that do not overlap. In one embodiment where the binding molecules are antibodies, it may be that the specificities bind non-identical epitopes of CD45. In one embodiment, the epitopes may overlap, but be non-identical. In another embodiment, they may not overlap at all. In one embodiment, two different specificities may be defined as ones that do not compete with each other for binding to CD45, or which do not cross-block each other, or which do not significantly do so. As discussed further below, one preferred way to determine whether the specificities for CD45 are different is to perform cross-blocking or competition assays. The binding molecules preferably do not compete or cross-block each other. They should typically both be able to bind CD45 at the same time, but at non-identical epitopes.

The number of binding sites that a binding molecule, in particular an antibody, has may be referred to as its valency, with each valency representing a binding site, and in the case of an antibody one antigen-binding site of the antibody. Each valency may represent the same or different specificity; for example a bispecific IgG antibody has a valency of two and two different specificities. In one embodiment, a binding molecule, and in particular an antibody, of the present invention may have at least two different specificities against CD45. It may, for instance, have two, three, four, five, six, seven, eight, nine, or ten different specificities for CD45. In one embodiment, a binding molecule of the present invention, in particular an antibody, may comprise two or three different specificities against CD45 and in particular at least two different antigen-binding sites conferring different specificities for CD45. In one embodiment, a binding molecule of the present invention, in particular an antibody of the present invention, comprises three different specificities against CD45. In an especially preferred embodiment, a binding molecule of the present invention, in particular an antibody of the present invention, comprises two different specificities against CD45. In one embodiment, an antibody may comprise at least two different paratopes, where each paratope is specific for a different epitope of CD45. In one embodiment, a binding molecule, and in particular an antibody, of the present invention has a valency of two and has two different specificities for CD45. In another embodiment, it has a valency of three and two of those valencies correspond to different specificities for CD45. In one embodiment, the other valency is a specificity for serum albumin.

In another embodiment, a binding molecule, and in particular an antibody, of the present invention, may have a valency of three, with each binding site of the molecule being specific for CD45. In one preferred embodiment, all three binding sites will have a different specificity for CD45. Hence, in one preferred embodiment, a binding molecule, and in particular an antibody of the invention may have three different specificities for CD45. Such a molecule may therefore have, for instance, three different paratopes for CD45. Hence, in some embodiments of the invention, binding molecules, and in particular antibodies, are multi-valent, and preferably are multi-specific for CD45. Thus, also provided are binding molecules which are multi-specific for CD45. In particular, they are provided and are multi-paratopic for CD45. For example, in one embodiment, a binding molecule, and in particular an antibody, may have three, four, or more different specificities for CD45 and in particular such a number of paratopes. In one preferred embodiment, it has two, three, or four different specificities for CD45. In particular, it may have such a number of different paratopes for CD45. In one particularly preferred embodiment, it has three different specificities for CD45, and preferably it has three different paratopes for CD45. In another preferred embodiment, as well as having such numbers of specificities/paratopes for CD45, a binding molecule, in particular an antibody, of the present invention also has at least one other specificity for an antigen which is not CD45 conferred by a separate binding site. For example, the binding molecule may also be able to bind to albumin through a binding site separate to those binding CD45.

In one particularly preferred embodiment, a binding molecule or molecules of the present invention, particularly an antibody of the present invention, can bind CD45, bringing about multimerisation of CD45. In one embodiment, a binding molecule of the present invention, particularly an antibody of the present invention, binds an extracellular portion of CD45 to bring about CD45 multimerisation. In an alternative embodiment, a binding molecule of the present invention, particularly an antibody of the present invention, may bind an intracellular portion of CD45. In a preferred embodiment, the binding molecule of the present invention, particularly an antibody of the present invention, may bind to an intracellular portion of CD45 bringing about multimerisation of CD45.

A binding molecule or molecules of the present invention may be used to multimerise CD45. In particular, they may be employed to multimerise CD45 on the surface of a target cell. CD45 multimers are in particular higher order structures of more than one CD45 molecule linked together via a binding molecule or molecules of the present invention. For example, in one embodiment a multimer of CD45 may comprise at least two CD45 molecules. In a particularly preferred embodiment, a multimer of CD45 comprises at least three CD45 molecules. In one embodiment, a multimer of CD45 may comprise at least three, four, five, six, seven, or more CD45 molecules joined together by binding molecules of the present invention. As discussed herein, techniques such as mass photometry may be used to identify multimers of CD45 complexed with binding molecules of the present invention and hence to gauge the ability of binding molecule(s) of the present invention to generate multimers of CD45. In one embodiment, a binding molecule or molecules of the present invention are used to cross-link CD45. In a preferred embodiment, a binding molecule or molecules of the present invention are used to cross-link CD45 molecules on the surface of a target cell. In a further embodiment, they bind to an internal portion of CD45 and cross-link CD45 that way, preferably generating multimers of CD45.

### Mixtures of Binding Molecules

In another embodiment, rather than a single binding molecule, a mixture of at least two different binding molecules may be provided. For example, in one embodiment, a mixture of at least two different binding molecules is provided where individual binding molecules in the mixture only have one specificity for CD45, but collectively the mixture of binding molecules has at least two different specificities for CD45. The use of a mixture of binding molecules therefore represents a further way to promote cross-linking of CD45. Mixtures of binding molecules, in particular mixtures of antibodies, where individual binding molecules of the mixture have at least two different specificities for CD45 are also provided. In another embodiment, mixtures of binding molecules which collectively have only one specificity for CD45 may be employed. Both a binding molecule and binding molecules of the present invention may be provided as a mixture together with other therapeutic agents.

Anywhere herein where reference to a binding molecule is made a mixture of binding molecules may alternatively be employed unless specifically stated. For example, anywhere herein that an individual antibody is referred to, a mixture of at least two different antibodies may alternatively be employed unless specifically stated otherwise. The converse is also the case.

### Screening for Biomolecules

As well as the binding molecules themselves, the present invention also provides methods for identifying binding molecules of the present invention and determining the efficacy of such binding molecules. Various functional assays are disclosed herein and they may be, for example, employed.

For example, the present invention provides a method of screening for a binding molecule or molecules able to multimerise CD45 to induce cell death, the method comprising: (a) contacting a binding molecule or molecules that are able to bind CD45 with target cells expressing CD45; and (b) determining whether the target cells undergo cell death. In one embodiment, the method further comprises: (c) determining whether cytokines are released in the test sample, for example where the level of one or more of CCL2, GM-CSF, IL-1RA, IL-6, IL-8, IL-10, IL-11, and M-CSF is measured. In one embodiment, the binding molecule or molecules have already been identified as able to multimerise CD45. In another embodiment, the method comprises first screening binding molecules specific for CD45 for their ability to multimerise CD45, for example by screening permutations of two or more different binding molecules for their ability to multimerise CD45.

In one embodiment, the present invention provides a method of identifying biomolecules that comprise at least two different specificities. For example, the method may comprise screening a library of pairwise permutations of specificities for CD45. In one embodiment, the pairwise permutations are screened for their ability to multimerise CD45, for example by mass photometry. In another embodiment, they are screened for their ability to bring about killing of target cells expressing CD45. In another embodiment, they are screened for their ability to kill such target cells whilst not triggering cytokine release. Various functional assays and screening formats are described herein and any of them may be used. In one embodiment, the Fab-X/Fab-Y format is used to screen pairwise combinations. In one embodiment, where pairwise permutations are being assessed, the screening may also comprise comparing to the equivalent molecule with just one such specificity.

In another embodiment, in order to identify desirable mixtures of at least two binding molecules, various permutations of mixtures of different individual binding molecules specific for CD45 may be screened for a desired property. In one embodiment, the screen also compares the activity of the mixture with that of the individual binding molecules. Hence, the present invention provides a method for identifying a mixture of binding molecules of the invention that are able to multimerise CD45, but not induce cytokines to a significant level, comprising screening mixtures comprising the various permutations of a panel of individual binding molecules specific for CD45, and identifying the mixture that gives the highest level of a desired property. For example, the assay may identify the mixture giving the highest level of multimerisation or alternatively the mixture giving the highest level of cell killing of target cells expressing CD45. The method may involve identifying the mixture that gives the highest level of cell killing without cytokine release.

### Antibodies

In an especially preferred embodiment, the binding molecule or molecules of the present invention is an antibody or antibodies against CD45. Thus, in any of the embodiments outlined herein where a binding molecule or binding molecules are referred to, preferably an antibody or antibodies are employed. The term “antibody” includes the various antibody formats disclosed herein, including those comprising various formats of heavy and/or light chains discussed herein. Thus, for instance, the term “antibody” specifically includes the Fab-X/Fab-Y, BYbe, TrYbe, and on-site multimerisation IgG antibody formats discussed herein. The term “antibody” also includes antibody fragments, preferably those mentioned herein. As discussed herein, one particularly preferred antibody isotype is IgG4.

In a particularly preferred embodiment, the sequence of an antibody is such that it favours heterodimer formation over homodimer formation, so that the antibody comprises two different heavy chains and hence two different specificities. Alternatively, it may have modifications that allow purification of heterodimeric antibody over homodimeric antibody. Such formats may be used in particular where the desired antibody is one with two different specificities and hence it is desired that the antibody has two different heavy chains, ones for each specificity.

As discussed above, a specificity of a binding molecule may denote the target to which the binding molecule binds and also where on the target the binding molecule binds. Hence, for an antibody it denotes the target an antigen-binding site of the antibody binds and where on the target it binds. In the context of an antibody, an antigen-binding site of the antibody may be said to confer a specificity of the antibody. Two antibodies may be said to have a different specificity for CD45 if they both bind CD45, but at non-identical locations. For example the locations may overlap, but be non-identical, or they may not overlap at all. A “paratope” of an antibody is a portion of an antibody antigen binding site that recognises and binds to an antigen. In particular, a paratope is a portion of an antibody that recognises and binds an epitope of an antigen. In one preferred embodiment, where two different specificities or paratopes are referred to, they will be different in the sense that each binds a different portion of CD45. In particular, each will bind a different epitope of CD45. In one embodiment, where different specificities or paratopes for CD45 are referred to it may mean different and in particular non-identical epitopes of CD45 are bound. Hence, in one preferred embodiment, the epitopes of CD45 bound are non-identical. In one embodiment, it denotes that the different specificities correspond to different paratopes for CD45.

In one embodiment, the specificities, and in particular the paratopes, of an antibody of the present invention each bind to different epitopes of CD45. Binding a “different epitope” means that the two epitopes are not identical. In one preferred embodiment, the two different epitopes recognised do not overlap at all. For example, in a preferred embodiment, the epitopes recognised are separated by at least one amino acid in the linear amino acid sequence of CD45. In another preferred embodiment, the two epitopes recognised are separated by at least five, ten, fifteen, twenty, fifty, 100 or more amino acids in the linear sequence of CD45. In another embodiment, the two different epitopes may overlap a small amount, for instance, by five or less amino acids in the linear sequence of CD45. In another embodiment, the epitopes may overlap by four or less amino acids, for example by three or less amino acids, preferably by two or less amino acids. In another preferred embodiment the epitopes will overlap by only a single amino acid or not at all in the linear sequence of CD45. In one embodiment, where the epitopes are non-linear, for example where they are conformational epitopes, it may be that there is some overlap in the portions of CD45 bound as the epitopes, but that the two portions of CD45 bound are not identical. In one embodiment, the conformational epitopes will not overlap at all.

In one embodiment, when it is desired to determine if two specificities for a binding molecule, and in particular an antibody, are different, a binding molecule having just one of the supposed specificities will be generated for each of the two specificities, preferably where the binding molecules have the same valency, but differ only in the specificities present. The ability of those two binding molecules to compete or cross-block in binding assays will be determined. In particular, such assays will determine if both binding molecules are able to bind CD45, but not reduce the binding of each other significantly to CD45. So, for instance, a cross-blocking or competition assay may in a preferred embodiment compare the binding of each binding molecule to CD45 individually, but also when both binding molecules are mixed together with CD45. In one embodiment, a desired antibody will not reduce the binding of the other.

For example, in the context of antibodies, antibodies having the same valency will be generated where the, or each, binding site of that antibody confers just one of the specificities. The ability for such antibodies for each specificity to compete or cross-block each other will be determined. The antibodies though should both still bind CD45. In one preferred embodiment, a monovalent antibody for each specificity will be generated, for example a scFv or Fab, and then the ability of the antibodies for each specificity to cross-block or compete measured. In another, a bivalent antibody for each specificity will be generated and the ability of each to compete or cross-block the other will be determined. In one preferred embodiment, the antibodies used in the comparison will be identical apart from the difference in the regions conferring the specificities, for example only having different variable regions, and in particular only differing in terms of the paratopes. For example the two antibodies may differ only in the different variable regions for the paratopes. In one embodiment no cross-blocking is seen when such a comparison is performed. In another embodiment, no significant cross-blocking is seen. For example, the amount of cross-blocking by one of the antibodies by the other may be less than 25%, preferably less than 20%, more preferably less than 10%. In another preferred embodiment, the degree of cross-blocking may be less than 5%. In another embodiment, the degree of cross-blocking will be less than 1%. In another preferred embodiment, 0% cross-blocking will be seen. These percentages refer to the extent to which a first antibody reduces binding of second antibody to CD45, for example in an ELISA.

In one embodiment, the affinity of the binding domain for CD45 in an antibody of the present invention is about 100 nM or stronger such as about 50 nM, 20 nM, 10 nM, 1 nM, 500 pM, 250 pM, 200 pM, 100 pM or stronger. In one embodiment, the binding affinity is 50 pM or stronger. In one embodiment, at least one paratope of the antibody has such an affinity for CD45. In another embodiment, the antibody has two paratopes, each having a different specificity for CD45, where all of the paratopes individually have such an affinity for CD45. In one embodiment, that is the overall avidity of the antibody for CD45. In one embodiment, the affinity of a paratope for CD45 may be less than 1 μM, less than 750 nM, less than 500 nM, less than 250 nM, less than 200 nM, less than 150 nM, less than 100 nM, less than 75 nM, less than 50 nM, less than 10 nM, less than 1 nM, less than 0.1 nM, less than 10 pM, less than 1 pM, or less than 0.1 pM. In some embodiments, the Kd is from about 0.1 pM to about 1 μM. In one embodiment, an antibody of the invention overall has that level of affinity for CD45. In one embodiment, a binding molecule of the present invention will show such affinity for CD45. In another embodiment, where a specificity is being referred to it will show such a value. In a further embodiment, a binding molecule of the present invention, or a specificity of a binding molecule, will show such values.

Where an antibody of the present invention has more than one specificity, in one embodiment the antibody is chosen to have particular specificities. For example, the different specificities may be chosen so that the binding sites for each have approximately similar affinities. For instance, the binding affinities for the individual specificities may be chosen to be within a factor of 100, preferably a factor of 50, and in particular within a factor of 10 of each other. In another embodiment, the different specificities of an antibody of the present invention may be chosen so that they have different affinities. For example, in one embodiment they may be at least 10-fold different from each other. In another embodiment, they may be at least 50-fold different from each other. In a further embodiment the affinities may be at least 100-fold different from each other. In another embodiment the affinities may be at least 1000-fold different. For example such levels of difference may be seen in the KD values.

In a preferred embodiment, an antibody of the present invention will have at least two specificities, in particular at least two different paratopes each binding different epitopes of CD45, and so may be in any suitable antibody format that allows that. Preferably, whilst neither antibody blocks the binding of the other significantly, both should still be able to bind CD45 at the same time. In embodiments where the antibody of the invention comprises at least two different paratopes specific for CD45, typically each paratope of the biparatopic antibody will be able to specifically bind CD45, with the two paratopes each specifically binding to a different epitope of CD45. Hence, the presence of different specificities will still though allow the simultaneous binding of both.

In one embodiment, where there are two variable regions in an antigen-binding site and/or in each antigen-binding site of an antibody, the two variable regions may work co-operatively to provide specificity for CD45, for example they are a cognate pair or affinity matured to provide adequate affinity such that the domain is specific to a particular antigen. Typically, they are a heavy and light chain variable region pair (VH/VL pair). In one embodiment, two different antigen-binding sites of an antibody of the present invention will each comprise the same light chain, also referred to as a “common” light chain. For instance, in one embodiment, an antibody of the invention is in the IgG antibody format and comprises such a common light chain. In one embodiment, such an approach may be combined with knobs-and-holes modifications in the heavy chains that favour heterodimer formation.

The antibodies of the present invention may comprise a complete antibody having full length heavy and light chains or a fragment thereof, for instance, a Fab, modified Fab, Fab′, modified Fab′, F(ab′)2, Fv, single domain antibody (e.g. VH or VL or VHH), scFv, bi, tri or tetra-valent antibody, Bis-scFv, diabody, triabody, tetrabody or epitope-binding fragments of any of the above (see for example Holliger and Hudson, 2005, Nature Biotech. 23(9):1126-1136; Adair and Lawson, 2005, Drug Design Reviews—Online 2(3), 209-217). In embodiments of the invention where a binding molecule, in particular an antibody, has a certain number of binding sites but the type of fragment or antibody format referred to has less than that number of binding sites it may still form part of the overall binding molecule. The methods for creating and manufacturing antibody fragments are well known in the art (see for example Verma et al., 1998, Journal of Immunological Methods, 216, 165-181). Other antibody fragments for use in the present invention include the Fab and Fab′ fragments described in International patent applications WO2005/003169, WO2005/003170 and WO2005/003171. Multi-valent antibodies may comprise multiple specificities e.g bispecific or may be monospecific (see for example WO 92/22853, WO05/113605, WO2009/040562 and WO2010/035012.

An antibody of the invention may be in any of the formats discussed herein. In one particularly preferred embodiment, an antibody of the invention is in the BYbe, TrYbe, or IgG antibody format. Such antibody formats are especially preferred in the various embodiments of the present invention where the antibody is being employed therapeutically.

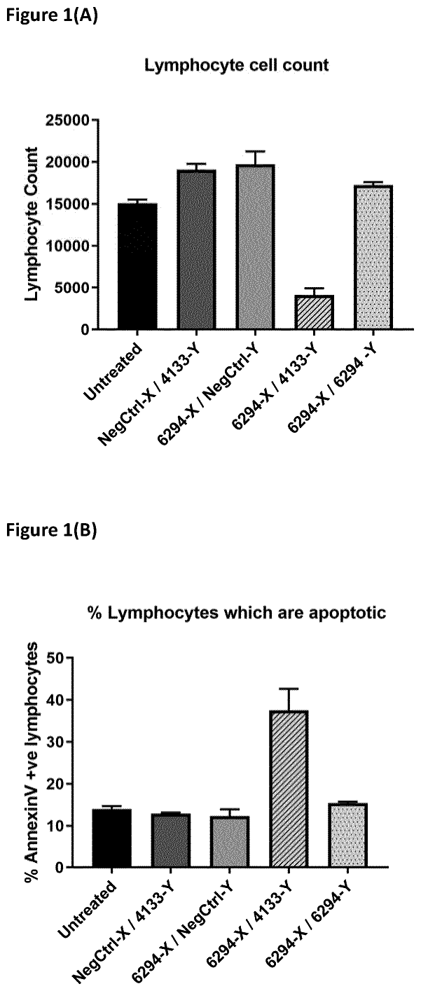

Examples of possible antibody formats are known in the art, for example as disclosed in the review “The coming of Age of Engineered Multivalent Antibodies, Nunez-Prado et al Drug Discovery Today Vol 20 Number 5 Mar. 2015, page 588-594, D. Holmes, Nature Rev Drug Disc November 2011:10; 798, Chan and Carter, Nature Reviews Immunology vol. 10, May 2010, 301 incorporated herein by reference. In one embodiment, an antibody of the invention may comprise, consist essentially of, or consist of any of the following formats:
tandem sdAb, tandem sdAb-sdAb (three sdAbs);
(scFv)₂ (also referred to as tandem scFv), scFv-dsFv, dsscFv-dsFv (dsFv)₂;
diabody, dsdiabody, didsdiabody;
scdiabody, dsscdiabody, didsscdiabody;
Dart antibody i.e, VL₁ linker VH₂ linker and VH₁ linker VL₂ wherein the C-terminous of VH₁ and VH₂ are joined by a disulphide bond;
BiTE®, dsBiTE, didsBiTE;
Di-diabody (see Nunez-Prado et al in particular molecule number 25 in FIG. 1 therein), dsdi-diabody, didsdi-diabody;
triabody, dstriabody, didstriabody, tridstriabody;
tetrabodies, dstetrabody, didstetrabody, tridstetrabody, tetradstetrabody; tandab (see Nunez-Prado et al in particular molecule number 22 in FIG. 1 therein);
dstandab, didstandab, tridstandab, tetradstandab;
a ByBe or TrYbe format antibody
[sc(Fv)₂]₂, (see Nunez-Prado et al in particular molecule number 22 in FIG. 1 therein), ds[sc(Fv)₂]₂, dids[sc(Fv)₂]₂, trids[sc(Fv)₂]₂, tetrads[sc(Fv)₂]₂;
Pentabody (see Nunez-Prado et al in particular molecule number 27 in FIG. 1 therein);
Fab-scFv (also referred to as a bibody), Fab′scFv, FabdsscFv (or BYbe),
Fab′dsscFv;
tribody, dstribody, didstribody (also referred to as FabdidsscFv or TrYbe or Fab-(dsscFv)₂), Fab′didsscFv;
Fabdab, FabFv, Fab′dab, Fab′Fv;
Fab single linker Fv (also referred to herein as FabdsFv as disclosed in WO2014/096390), Fab′ single linker Fv (also referred to herein as Fab′dsFv);
FabscFv single linker Fv, Fab′scFv single linker Fv;
FabdsscFv single linker Fv, Fab′dsscFv single linker Fv;
FvFabFv, FvFab′Fv, dsFvFabFv, dsFvFab′Fv, FvFabdsFv, FvFab′dsFv, dsFvFabdsFv, dsFvFab′dsFv;
FabFvFv, Fab′FvFv, FabdsFvFv, Fab′dsFvFv, FabFvdsFv, Fab′FvdsFv, FabdsFvdsFv, Fab′dsFvdsFv;
diFab, diFab′ including a chemically conjugated diFab′;
(FabscFv)₂, (Fab)₂scFvdsFv, (Fab)₂dsscFvdsFv, (FabdscFv)₂;
(Fab′scFv)₂, (Fab′)₂scFvdsFv, (Fab′)₂dsscFvdsFv, (Fab′dscFv)₂;
V_{H}HC_{K} (see Nunez-Prado et al in particular molecule number 6 in FIG. 1 therein);
minibody, dsminibody, didsminibody;
a miniantibody (ZIP) [see Nunez-Prado et al in particular molecule number 7 in FIG. 1 therein], dsminiantibody (ZIP) and didsminiantibody (ZIP);
tribi-minibody [see Nunez-Prado et al in particular molecule number 15 in FIG. 1 therein] dstribi-minibody, didstribi-minibody, tridstribi-minibody;
diabody-CH₃, dsdiabody-CH₃, didsdiabody-CH₃, scdiabody-CH₃, dsscdiabody-CH₃, didsscdiabody-CH₃;
tandemscFv-CH₃, tandemdsscFv-CH₃, tandemdidsscFv-CH₃, tandemtridsscFv-CH₃, tandemtetradsscFv-CH₃,
scorpion molecule (Trubion) i.e. a binding domain, linker —CH₂CH₃ binding domain as described in U.S. Pat. No. 8,409,577;
SMIP (Trubion) i.e. (scFv-CH₂CH₃)₂;
(dsFvCH₂CH₃)₂, tandem scFv-Fc, tandem dsscFvscFv-Fc, tandem dsscFv-Fc, scFv-Fc-scFv, dsscFv-Fc-scFv, scFv-Fc-dsscFv;
diabody-Fc, dsdiabody-Fc, didsdiabody-Fc, triabody-Fc, dstriabody-Fc, didstriabody-Fc, tridstriabody-Fc, tetrabody-Fc, dstetrabody-Fc, didstetrabody-Fc, tridstetrabody-Fc, tetradstetrabody-Fc, dstetrabody-Fc, didstetrabody-Fc, tridstetrabody-Fc, tetradstetrabody-Fc, scdiabody-Fc, dsscdiabody, didsscdiabody;
bi or trifunctional antibody, for example with different heavy chain variable regions and common light chains for example Merus bispecific antibody format (Biclonics®) with common light chains of a fixed sequence and different heavy chains (including different CDRs) and engineered CH₃ domain to drive the dimerization o the different heavy chains;
Duobody (i.e. wherein one full length chain in the antibody has different specificity to the other full length chain in the antibody);
a full-length antibody wherein Fab arm exchange has been employed to create a bispecific format;
bi or tri functional antibody, wherein a full-length antibody has common heavy chain and different light chains also referred to as kappa/lambda body’ or ‘κ/λ-body, see for example WO2012/023053 incorporated herein by reference;
Ig-scFv one, two, three or four from the C terminus of heavy or light chain, scFv-Ig one, two, three or four from the N terminus of heavy or light chain, single linker Ig-Fv, Ig-dsscFv one, two, three or four from the C terminus of heavy or light chain (with one, two, three or four disulfide bonds);
Ig-dsscFv one, two, three or four from the N terminus of heavy or light chain (with one, two, three or four disulfide bonds);
Ig single linker Fv (see PCT/EP2015/064450);
Ig-dab, dab-Ig, scFv-Ig, V-Ig, Ig-V;
scFabFvFc, scFabdsFvFc (single linker version scFavFv), (FabFvFc)₂, (FabdsFvFc)₂, scFab′FvFc, scFab′dsFvFc, (Fab′FvFc)₂, (Fab′dsFvFc)₂; and
DVDIg, which are discussed in more detail below.

In one embodiment antibody formats include those known in the art and those described herein, such as wherein the antibody molecule format is, or comprises, one of those selected from the group comprising or consisting of: diabody, BYbe, scdiabody, triabody, tribody, tetrabodies, TrYbe, tandem scFv, FabFv, Fab′Fv, FabdsFv, Fab-scFv, Fab-dsscFv, Fab-(dsscFv)₂, diFab, diFab′, tandem scFv-Fc, scFv-Fc-scFv, scdiabody-Fc, scdiabody-CH₃, Ig-scFv, scFv-Ig, V-Ig, Ig-V, Duobody and DVDIg, which are discussed in more detail below. A further preferred antibody format for employing in the present invention is a bispecific antibody.

In one preferred embodiment the antibody molecule of the present invention does not comprise an Fc domain, i.e. does not comprise a CH₂ and CH₃ domain. For example, the molecule may be selected from the group comprising a tandem scFv, scFv-dsFv, dsscFv-dsFv didsFv, diabody, dsdiabody, didsdiabody, scdiabody (also referred to as an (scFv)₂), dsscdiabody, triabody, dstriabody, didstriabody, tridstriabody, tetrabodies, dstetrabody, didstetrabody, tridstetrabody, tetradstetrabody, tribody, dstribody, didstribody, Fabdab, FabFv, Fab′dab, Fab′Fv, Fab single linker Fv (as disclosed in WO2014/096390), Fab′ single linker Fv, FabdsFv, Fab′dsFv, Fab-scFv (also referred to as a bibody), Fab′scFv, FabdsscFv, Fab′dsscFv, FabdidsscFv, Fab′didsscFv, FabscFv single linker Fv, Fab′scFv single linker Fv, FabdsscFvs single linker Fv, Fab′dsscFv single linker Fv, FvFabFv, FvFab′Fv, dsFvFabFv, dsFvFab′Fv, FvFabdsFv, FvFab′dsFv, dsFvFabdsFv, dsFvFab′dsFv, FabFvFv, Fab′FvFv, FabdsFvFv, Fab′dsFvFv, FabFvdsFv, Fab′FvdsFv, FabdsFvdsFv, Fab′dsFvdsFv, diFab, diFab′ including a chemically conjugated diFab′, (FabscFv)₂, (Fab)₂scFvdsFv, (Fab)₂dsscFvdsFv, (FabdscFv)₂, minibody, dsminibody, didsminibody, diabody-CH₃, dsdiabody-CH₃, didsdiabody-CH₃, scdiabody-CH₃, dsscdiabody-CH₃, didsscdiabody-CH₃, tandemscFv-CH₃, tandemdsscFv-CH₃, tandemdidsscFv-CH₃, tandemtridsscFv-CH₃ and tandemtetradsscFv-CH₃. In one embodiment, an antibody of the invention is, or comprises, a diabody. In another embodiment it is, or comprises, a duobody.

The following provides further explanation of antibody formats suitable for use in the present invention either as the antibody of the present invention or as part of the overall antibody:
“Single domain antibody” (also referred to herein as a dab and sdAb) as used herein refers to an antibody fragment consisting of a single monomeric variable antibody domain. Examples of single domain antibodies include VH or VL or VHH.
Tandem-sdAb as employed herein refers to two domain antibodies connected by a linker, for example a peptide linker, in particular where the domain antibodies have specificity for different antigens.
Tandem-sdAb-sdAb as employed herein refers to three domain antibodies connected in series by two linkers, for example peptide linkers, in particular where the domain antibodies have specificity for different antigens.
dsFv as employed herein refers to an Fv with an intra-variable disulfide bond. The dsFv may be a component of a larger molecule, for example one of the variable domains may be linked, for example via an amino acid linker to another antibody fragment/component.
(dsFv)₂ as employed herein refers to a dsFv with one domain linked, for example via a peptide linker or a disulfide bond (for example between, the C-terminus of two V_{H}'s) to a domain in a second dsFv, the format resembles a (scFv)₂ described below but each pair of variable regions comprise a intra-variable region disulfide bond.
   Component as employed herein refers to a building block or portion of an antibody of the present invention, in particular where the component is an antibody fragment such as scFv, Fab or other fragment, in particular as described herein, it may be used, in some embodiments, as part of the overall antibody of the present invention.
Single-chain Fv or abbreviated as “scFv”, as used herein refers to an antibody fragment that comprises VH and VL antibody domains linked (for example by a peptide linker) to form a single polypeptide chain. The constant regions of the heavy and light chain are omitted in this format.
dsscFv as employed herein refers to scFv with an intra-variable region disulfide bond.
Tandem scFv (also referred to herein as a discFv or (scFv)₂)) as employed herein refers to two scFvs linked via a single linker such that there is a single inter-Fv linker.
Tandem dsscFv (also referred to herein as a scFvdsscFv or dsscFvscFv) as employed herein refers to two scFvs linked via a single linker such that there is a single inter-Fv linker, and wherein one of the scFv has an intravariable region disulfide bond.
Tandem didsscFv (also referred to herein as a didsscFv) as employed herein refers to two scFvs linked via a single linker such that there is a single inter-Fv linker, and wherein each scFv comprises an intravariable region disulfide bond.
scFv-dsFv as employed herein is a scFv linked, for example by a peptide linker, to an Fv domain which is comprised of two variable domains linked via a disulfide bond to form a dsFv. In this format the VH or VL of the scFv may be linked to the VH or VL of the dsFv.
dsscFv-dsFv as employed herein is a dsscFv linked, for example by a peptide linker, to an Fv domain which is comprised of two variable domains linked via a disulfide bond to form a dsFv. In this format the VH or VL of the dsscFv may be linked to the VH or VL of the dsFv.
Diabody as employed herein refers to two Fv pairs VH/VL and a further VH/VL pair which have two inter-Fv linkers, such that the VH of a first Fv is linked to the VL of the second Fv and the VL of the first Fv is linked to the VH of the second Fv.
dsDiabody as employed herein refers to a diabody comprising an intra-variable region disulfide bond.
didsDiabody as employed herein refers to a diabody comprising two intra-variable region disulfide bonds, i.e. one ds between each pair of variable regions.
Sc-diabody as employed herein refers a diabody comprising an intra-Fv linker, such that the molecule comprises three linkers and forms two normal scFvs, for example VHi₁inkerVL₁ linker VH₂ linker VL₂
dssc-diabody as employed herein refers to a sc-diabody with an intra-variable region disulfide bond.
didssc-diabody as employed herein refers to a sc-diabody with an intra-variable region disulfide bond between each pair of variable regions.
Dart as employed herein refers to VL₁ linker VH₂ linker and VH₁ linker VL₂ wherein the C-terminous of VH₁ and VH₂ are joined by a disulfide bond Paul A. Moore et al Blood, 2011; 117(17):4542-4551.
Bite® as employed herein refers to a molecule comprising two pairs of variable domains in the following format; a domain from pair 1 (e.g. VH₁) connected via a linker to a domain from pair 2 (e.g. VH₂ or VL₂) said second domain connected by a linker to the further domain from pair 1 (e.g. VL₁) in turn connected to the remaining domain from pair two (i.e. VL₂ or VH₂).
Di-diabody see Nunez-Prado et al in particular molecule number 25 in FIG. 1 therein.
Dsdi-diabody as employed herein is a di-diabody with an intra-variable region disulfide bond.
Didsdi-diabody as employed herein is a di-diabody with an intra-variable region disulfide bond between each pair of variable regions.
Triabody as employed herein refers to a format similar to the diabody comprising three Fvs and three inter-Fv linkers.
dstriabody as employed herein refers to a triabody comprising an intra-variable region disulfide bond between one of the variable domain pairs.
Didstriabody as employed herein refers to a triabody comprising two intra-variable region disulfide bonds, i.e. one ds between each of two variable domain pairs.
Tridstriabody as employed herein refers to a triabody comprising three intra-variable region disulfide bonds i.e. one ds between each pair of variable regions.
Tetrabody as employed herein refers to a format similar to the diabody comprising four Fvs and four inter-Fv linkers.
dstetrabody as employed herein refers to a tetrabody comprising an intra-variable region disulfide bond between one of the variable domain pairs.
Didstetrabody as employed herein refers to a tetrabody comprising two intra-variable region disulfide bonds, i.e. one ds between each of two variable domain pairs.
Tridstetrabody as employed herein refers to a tetrabody comprising three intra-variable region disulfide bonds i.e. one ds between each of three pairs of variable regions.
Tetradstetrabody as employed herein refers to a tetrabody comprising four intra-variable region disulfide bonds i.e. one ds between each variable domain.
Tribody (also referred to a Fab(scFv)₂) as employed herein refers to a Fab fragment with a first scFv appended to the C-terminal of the light chain and a second scFv appended to the C-terminal of the heavy the chain.
dstribody as employed herein refers to a tribody comprising a dsscFv in one of the two positions.
didstribody or TrYbe as employed herein refers to a tribody comprising two dsscFvs.
dsFab as employed herein refers to a Fab with an intra-variable region disulfide bond.
dsFab′ as employed herein referst to a Fab′ with an intra-variable region disulfide bond.
scFab is a single chain Fab fragment.
scFab′ is a single chain Fab′ fragment.
dsscFab is a dsFab as a single chain.
dsscFab′ is a dsFab′ as a single chain.
Fabdab as employed herein refers to a Fab fragment with a domain antibody appended to the heavy or light chain thereof, optionally via a linker.
Fab′dab as employed herein refers to a Fab′ fragment with a domain antibody appended to the heavy or light chain thereof, optionally via a linker.
FabFv as employed herein refers to a Fab fragment with an additional variable region appended to the C-terminal of each of the following, the CH₁ of the heavy chain and CL of the light chain see for example WO2009/040562. The format may be provided as a PEGylated version thereof see for example WO2011/061492,
Fab′Fv as employed herein is similar to FabFv, wherein the Fab portion is replaced by a Fab′. The format may be provided as a PEGylated version thereof.
FabdsFv as employed herein refers to a FabFv wherein an intra-Fv disulfide bond stabilises the appended C-terminal variable regions, see for example WO2010/035012. The format may be provided as a PEGylated version thereof
Fab single linker Fv and Fab′ single linker as employed herein refers to a Fab or Fab′ fragment linked to a variable domain, for example by a peptide linker, and said variable domain is linked to a second variable domain via an intra-variable domain disulfide bond thereby forming a dsFv, see for example WO2014/096390.
Fab-scFv (also referred to as a bibody) as employed herein is a Fab molecule with a scFv appended on the C-terminal of the light or heavy chain, optionally via a linker.
Fab′-scFv as employed herein is a Fab′ molecule with a scFv appended on the C-terminal of the light or heavy chain, optionally via a linker.
FabdsscFv or BYbe as employed herein is a FabscFv with a disulfide bond between the variable regions of the single chain Fv.
Fab′dsscFv as employed herein is a Fab′scFv with a disulfide bond between the variable regions of the single chain Fv.
FabscFv-dab as employed herein refers to a Fab with a scFv appended to the C-terminal of one chain and domain antibody appended to the C-terminal of the other chain.
Fab′scFv-dab as employed herein refers to a Fab′ with a scFv appended to the C-terminal of one chain and domain antibody appended to the C-terminal of the other chain.
FabdsscFv-dab as employed herein refers to a Fab with a dsscFv appended to the C-terminal of one chain and domain antibody appended to the C-terminal of the other chain.
Fab′dsscFv-dab as employed herein refers to a Fab′ with a dsscFv appended to the C-terminal of one chain and domain antibody appended to the C-terminal of the other chain.
FabscFv single linker Fv as employed herein refers to a Fab single linker Fv wherein a domain of the Fv is linked to the heavy or light chain of the Fab and a scFv is linked to the other Fab chain and the domains of the Fv are connected by an intra-variable region disulfide.
FabdsscFv single linker Fv as employed herein refers to a FabscFv single linker Fv wherein the scFv comprises an intra-variable region disulfide bond.
Fab′scFv single linker Fv as employed herein refers to a Fab′ single linker Fv wherein a domain of the Fv is linked to the heavy or light chain of the Fab and a scFv is linked to the other Fab chain and the domains of the Fv are connected by an intra-variable region disulfide.
Fab′dsscFv single linker Fv as employed herein refers to a Fab′scFv single linker Fv wherein the scFv comprises an intra-variable region disulfide bond.
FvFabFv as employed herein refers to a Fab with the domains of a first Fv appended to the N-terminus of the heavy and light chain of the Fab and the domains of a second Fv appended to the C-terminus of the heavy and light chain.
FvFab′Fv as employed herein refers to a Fab′ with the domains of a first Fv appended to the N-terminus of the heavy and light chain of the Fab′ and the domains of a second Fv appended to the C-terminus of the heavy and light chain.
dsFvFabFv as employed herein refers to a Fab with the domains of a first Fv appended to the N-terminus of the heavy and light chain of the Fab wherein the first Fv comprises an intra-variable region disulfide bond and the domains of a second Fv appended to the C-terminus of the heavy and light chain.
FvFabdsFv as employed herein refers to a Fab with the domains of a first Fv appended to the N-terminus of the heavy and light chain of the Fab and the domains of a second Fv appended to the C-terminus of the heavy and light chain and wherein the second Fv comprises an intra-variable region disulfide bond.
dsFvFab′Fv as employed herein refers to a Fab′ with the domains of a first Fv appended to the N-terminus of the heavy and light chain of the Fab′ wherein the first Fv comprises an intra-variable region disulfide bond and the domains of a second Fv appended to the C-terminus of the heavy and light chain.
FvFab′dsFv as employed herein refers to a Fab′ with the domains of a first Fv appended to the N-terminus of the heavy and light chain of the Fab′ and the domains of a second Fv appended to the C-terminus of the heavy and light chain and wherein the second Fv comprises an intra-variable region disulfide bond.
dsFvFabdsFv as employed herein refers to a Fab with the domains of a first Fv appended to the N-terminus of the heavy and light chain of the Fab wherein the first Fv comprises an intra-variable region disulfide bond and the domains of a second Fv appended to the C-terminus of the heavy and light chain and wherein the second Fv also comprises an intra-variable region disulfide bond.
dsFvFab′dsFv as employed herein refers to a Fab′ with the domains of a first Fv appended to the N-terminus of the heavy and light chain of the Fab′ wherein the first Fv comprises an intra-variable region disulfide bond and the domains of a second Fv appended to the C-terminus of the heavy and light chain and wherein the second Fv also comprises an intra-variable region disulfide bond.
FabFvFv as employed herein refers to a Fab fragment with two pairs of Fvs appended in series to the C-terminal of the heavy and light chain, see for example WO2011/086091.
Fab′FvFv as employed herein refers to a Fab′ fragment with two pairs of Fvs appended in series to the C-terminal of the heavy and light chain, see for example WO2011/086091.
FabdsFvFv as employed herein refers to a Fab fragment with two pairs of Fvs appended in series to the C-terminal of the heavy and light chain, see for example WO2011/086091, wherein the first Fv pair attached directly to the C-terminal comprise an intra-variable region disulfide bond.
Fab′dsFvFv as employed herein refers to a Fab′ fragment with two pairs of Fvs appended in series to the C-terminal of the heavy and light chain, see for example WO2011/086091, wherein the first Fv pair attached directly to the C-terminal comprise an intra-variable region disulfide bond.
FabFvdsFv as employed herein refers to a Fab fragment with two pairs of Fvs appended in series to the C-terminal of the heavy and light chain, wherein the second Fv pair at the “C”-terminal of the molecule comprise an intra-variable region disulfide bond.
Fab′FvdsFv as employed herein refers to a Fab′ fragment with two pairs of Fvs appended in series to the C-terminal of the heavy and light chain, wherein the second Fv pair at the “C”-terminal of the molecule comprise an intra-variable region disulfide bond.
FabdsFvdsFv as employed herein refers to a Fab fragment with two pairs of Fvs appended in series to the C-terminal of the heavy and light chain, wherein the first and second Fv pair comprise an intra-variable region disulfide bond.
Fab′ dsFvdsFv as employed herein refers to a Fab′ fragment with two pairs of Fvs appended in series to the C-terminal of the heavy and light chain, wherein the first and second Fv comprise an intra-variable region disulfide bond.
DiFab as employed herein refers to two Fab molecules linked via their C-terminus of the heavy chains.
DiFab′ as employed herein refers to two Fab′ molecules linked via one or more disulfide bonds in the hinge region thereof.
DiFab and DiFab′ molecules include chemically conjugated forms thereof.
(FabscFv)₂ as employed herein refers to a diFab molecule with two scFvs appended thereto, for example appended to the C-terminal of the heavy or light chain, such as the heavy chain.
(Fab′scFv)₂ as employed herein refers to a diFab′ molecule with two scFvs appended thereto, for example appended to the C-terminal of the heavy or light chain, such as the heavy chain.
(Fab)₂scFvdsFv as employed herein refers to a diFab with a scFv and dsFv appended, for example one from each of the heavy chain C-terminal.
(Fab′)₂scFvdsFv as employed herein refers to a diFab′ with a scFv and dsFv appended, for example one from each of the heavy chain C-terminal.
(Fab)₂dsscFvdsFv, as employed herein refers to a diFab with a dsscFv and dsFv appended, for example from the heavy chain C-terminal.
(Fab′)₂dsscFvdsFv as employed herein refers to the a diFab′ with a dsscFv and dsFv appended, for example from the heavy chain C-terminal.
Minibody as employed herein refers to (VL/VH-CH₃)₂.
dsminibody as employed herein refers to (VL/VH-CH₃)₂ wherein one VL/VH comprises an intra-variable region disulfide bond.
didsminibody as employed herein refers to a (dsFv-CH₃)₂
scFv-Fc as employed herein refers to a scFv appended to the N-terminus of a CH₂ domain, for example via a hinge, of constant region fragment —(CH₂CH₃), such that the molecule has 2 binding domains.
dsscFv-Fc as employed herein refers to a dsscFv appended to the N-terminus of a CH₂ domain and a scFv appended to the N-terminus of a second CH₂ domain, for example via a hinge, of constant region fragment —(CH₂CH₃)₂, such that the molecule has 2 binding domains.
didsscFv-Fc as employed herein refers to a scFv appended to the N-terminus of a CH₂ domain, for example via a hinge, of constant region fragment —(CH₂CH₃)₂, such that the molecule has 2 binding domains
Tandem scFv-Fc as employed herein refers to two tandem scFvs, wherein each one is appended in series to the N-terminus of a CH₂ domain, for example via a hinge, of constant region fragment —(CH₂CH₃), such that the molecule has 4 binding domains.
Scdiabody-Fc as employed herein is two scdiabodies, wherein each one is appended to the N-terminus of a CH₂ domain, for example via a hinge, of constant region fragment —CH₂CH₃.
ScFv-Fc-scFv as employed herein refers to four scFvs, wherein one of each is appended to the N-terminus and the C-terminus of both the heavy and light chain of a —CH₂CH₃ fragment.
Scdiabody-CH₃ as employed herein refers to two scdiabody molecules each linked, for example via a hinge to a CH₃ domain.
kappa/lambda body’ or ‘κ/λ-body is in the format of a normal IgG with two heavy chains and two light chains, wherein the two light chains are different to each other, one is a lambda light chain (VL-CL) and the other is a kappa light chain (VK-CK). The heavy chain is identical, even at the CDRs as described in WO2012/023053.
IgG-scFv as employed herein is a full length antibody with a scFv on the C-terminal of each of the heavy chains or each of the light chains.
scFv-IgG as employed herein is a full length antibody with a scFv on the N-terminal of each of the heavy chains or each of the light chains.
V-IgG as employed herein is a full length antibody with a variable domain on the N-terminal of each of the heavy chains or each of the light chains.
IgG-V as employed herein is a full length antibody with a variable domain on the C-terminal of each of the heavy chains or each of the light chains
DVD-Ig (also known as dual V domain IgG) is a full length antibody with 4 additional variable domains, one on the N-terminus of each heavy and each light chain.
Duobody or ‘Fab-arm exchange’ as employed herein is a bispecific IgG format antibody where matched and complementary engineered amino acid changes in the constant domains (typically CH3) of two different monoclonal antibodies lead, upon mixing, to the formation of heterodimers. A heavy:light chain pair from the first antibody will, as a result of the residue engineering, prefer to associate with a heavy:light chain pair of a second antibody. See for example WO2008/119353, WO2011/131746 and WO2013/060867

An antibody of the present invention may be an antibody fragment, hence reference herein to an antibody also includes antibody fragments. In one embodiment, an antibody of the present invention may be any of the antibody fragments disclosed herein that comprises at least two different paratopes against CD45. In another embodiment, an antibody of the present invention may comprise an antibody fragment discussed herein that comprises only a single antigen-binding site against CD45, but be employed either as part of a binding molecule of the invention, or one of a mixture of antibodies as discussed herein. In another embodiment, monovalent antibody fragments may be employed in the present invention, preferably in antibody mixtures as set out herein. A “binding fragment” as employed herein refers to a fragment capable of binding a target peptide or antigen with sufficient affinity to characterise the fragment as specific for the peptide or antigen.

The term “Fab fragment” as used herein refers to an antibody fragment comprising a light chain fragment comprising a V_{L} (variable light) domain and a constant domain of a light chain (C_{L}), and a V_{H} (variable heavy) domain and a first constant domain (CH₁) of a heavy chain. The term “Fv” refers to two variable domains, for example co-operative variable domains, such as a cognate pair or affinity matured variable domains, i.e. a V_{H} and V_{L} pair. In one embodiment such fragments are used as an antibody molecule of the present invention. Co-operative variable domains as employed herein are variable domains that complement each other and/or both contribute to antigen binding to render the Fv (V_{H}/V_{L} pair) specific for the antigen in question.

An antibody of the present invention may comprise any of the antibody formats discussed herein, including in particular the Fab-X/Fab-Y, ByBe, TrYbe, and IgG formats discussed herein. BYbe, TrYbe, and IgG format antibodies are particularly useful in therapy. An antibody of the invention may comprise formats comprise heavy and/or light chain variable regions and, optionally linkers or other entities joining together different portions of the antibody. Such antibodies may be also referred to as molecules. In one particularly preferred embodiment, the antibody of the invention is in the IgG format. In another particularly preferred embodiment, the antibody of the invention is in the BYbe format. In another particularly preferred embodiment, an antibody of the invention is in the TrYbe format.

An antibody of the invention may also be an IgA, IgE, IgD, or IgM class antibody.

A degree of specificity (or specific) for a target molecule, in particular for CD45, as employed herein may refer to where the partners or a relevant part thereof in the interaction only recognise each other or have significantly higher affinity for each other in comparison to non-partners, for example at least 10 times, at least 100 times, at least 1000 times, at least 10,000 times, at least 100,000 times or at least 1,000,000 times higher affinity than for example a background level of binding or binding to another unrelated protein (e.g. hen egg white lysozyme). In one embodiment, such degrees of specificity are for CD45. In another embodiment, such specificity is not only for CD45, but also for particular a epitope of CD45 bound by an antigen binding site, and in particular a paratope, of the antibody, as compared to other epitopes of CD45.

A ‘binding site’ as employed herein refers to a binding region, typically a polypeptide, capable of binding a target antigen, for example with sufficient affinity to characterise the site as specific for the antigen. In a preferred embodiment, a binding site binds CD45. In one embodiment the binding site contains at least one variable domain or a derivative thereof, for example a pair of variable domains or derivatives thereof, such as a cognate pair of variable domains or a derivative thereof. Typically this is a VH/VL pair.

Any suitable antigen binding site may be used in the antibodies of the present invention. In one embodiment a binding site, in particular the paratope, contains at least one variable domain or a derivative thereof, for example a pair of variable domains or derivatives thereof, such as a cognate pair of variable domains or a derivative thereof. Typically this is a VH/VL pair.

Variable regions (also referred to herein as variable domains) generally comprise 3 CDRs and a suitable framework. In one embodiment, an antigen-binding site comprises two variable regions, a light chain variable region and a heavy chain variable region and together these elements contribute to the specificity of the binding interaction of the antibody or binding fragment for CD45 and in particular for the specificity in terms of where on CD45 the binding site binds. In one embodiment, the variable domains employed in an antigen binding site of an antibody molecule of the present invention are a cognate pair. A “cognate pair” as employed herein refers to a heavy and light chain pair of variable domains (or a derivative thereof, such as a humanised version thereof) isolated from a host as a pre-formed couple. This definition does not include variable domains isolated from a library, wherein the original pairing from a host is not retained. Cognate pairs may be advantageous because they are often affinity matured in the host and therefore may have higher affinity for the antigen to which they are specific, than a combination of variable domain pairs selected from a library, such as phage library. In another embodiment, the heavy and light chain in a binding site of an antibody of the present invention may not be a cognate pair. In one embodiment, for instance where a common light chain is used, the light chain is not cognate with at least one of the heavy chain variable regions, but is still able to form a functional antigen-binding site.

### Derivatives, Modifications, and Humanization

A “derivative” as employed herein is intended to refer to where one, two, three, four or five amino acids in a naturally occurring sequence have been replaced or deleted, for example to optimize properties such as by eliminating undesirable properties but wherein the characterizing feature(s) is/are retained. Examples of modifications are those to remove glycosylation sites, GPI anchors, or solvent exposed lysines. These modifications can be achieved by replacing the relevant amino acid residues with a conservative amino acid substitution.

Other modification in the CDRs may, for example, include replacing one or more cysteines with, for example a serine residue. Asn can be the substrate for deamination and this propensity can be reduced by replacing Asn and/or a neighbouring amino acid with an alternative amino acid, such as a conservative substitution. The amino acid Asp in the CDRs may be subject to isomerization. The latter can be minimized by replacing Asp and/or a neighboring amino acid with an alternative amino acid, for example a conservative substitution.

In one embodiment, a variable region or variable regions, for example in an antigen-binding site in an antibody molecule of the present invention, are humanized. Humanised versions of a variable region are also a derivative thereof, in the context of the present specification. Humanisation may include the replacement of a non-human framework for a human framework and optionally the back-mutation of one or more residues to “donor residues”. Donor residues as employed herein refers to residues found in the original variable region isolated from the host, in particular replacing a given amino acid in the human framework with the amino acid in the corresponding location in the donor framework. In one embodiment, any non-human variable region disclosed herein may also be present in an antibody molecule of the invention in humanized form. In one embodiment, CDRs as disclosed herein are present in human variable region frameworks. In another embodiment, framework donor residues may also be transferred as well as the CDRs. In another embodiment, an antibody of the present invention comprises fully human variable regions. In another embodiment, an antibody of the present invention is fully human.

### Antibody Constant Regions and Fc Region Functions

In one preferred embodiment, an antibody of the present invention does not comprise an Fc domain.

In one embodiment, an antibody of the present invention comprises an altered Fc domain as described herein below. In another preferred embodiment an antibody of the present invention comprises an Fc domain, but the sequence of the Fc domain has been altered to remove one or more Fc effector functions. In another embodiment, the Fc region of an antibody of the present invention has been modified to optimise a particular property of the antibody, such as any of those discussed herein.

In one embodiment, an antibody of the present invention comprises a “silenced” Fc region. For example, in one embodiment an antibody of the present invention does not display the effector function or functions associated with a normal Fc region.

Fc domain as employed herein generally refers to —(CH₂CH₃)₂, unless the context clearly indicates otherwise.

In one embodiment, an antibody of the present invention does not comprise a —CH₂CH₃ fragment.

In one embodiment, an antibody of the present invention does not comprise a CH₂ domain.

In one embodiment, an antibody of the present invention does not comprise a CH₃ domain.

In one embodiment, an antibody of the present invention does not bind Fc receptors.

In one embodiment, an antibody of the present invention does not bind complement. In one preferred embodiment, an antibody of the present invention does not bind the first complement factor, C1q or C1. In one embodiment, an antibody of the invention does not bind those factors because, for example, it lacks an Fc region. In another embodiment, an antibody of the present invention does not bind those factors because it has a modification in the constant region preventing its ability to do so. In an alternative embodiment, an antibody of the invention does not bind FcγR, but does bind complement. For example, in one embodiment, an antibody of the invention does not bind FcγR, but does bind C1q and/or C1.

In one embodiment the antibody of the present invention does not comprise an active Fc region in the sense that the antibody does not trigger the release of one or more cytokines which a normal Fc region would trigger the release of. For instance, the Fc region of an antibody of the invention may not trigger the release of cytokines when it binds to an Fc receptor or may not significantly do so.

In one embodiment, binding molecules of the present invention in general may comprise modifications that alter serum half-life of the binding molecule. Hence, in another embodiment, an antibody of the present invention has Fc region modification(s) that alter the half-life of the antibody. Such modifications may be present as well as those that alter Fc functions. In one preferred embodiment, an antibody of the present invention has modification(s) that alter the serum half-life of the antibody. In one particularly preferred embodiment, an antibody of the present invention has modification(s) that decrease serum half-life of the antibody compared to an antibody lacking such modifications. In another preferred embodiment, an antibody of the present invention comprises modification(s) that collectively both silence the Fc region and decrease the serum half-life of the antibody compared to an antibody lacking such modifications.

The antibody constant region domains of an antibody molecule of the present invention, if present, may be selected having regard to the proposed function of the antibody molecule, and in particular the effector functions which may be required. In a preferred embodiment, an antibody is one that lacks an Fc or lacks one or more effector function of an Fc region and preferably all of them. In other embodiments of the invention, the effector function(s) of the Fc region of the antibody may be still present. In one embodiment, an antibody of the invention may comprise a human constant region, for instance IgA, IgD, IgE, IgG or IgM domains. In particular, human IgG constant region domains may be used, especially of the IgG1 and IgG3 isotypes when the antibody molecule is intended for therapeutic uses where antibody effector functions are required. Alternatively, IgG2 and IgG4 isotypes may be used when the antibody molecule is intended for therapeutic purposes and antibody effector functions are not required. Particularly preferred IgG isotypes are IgG2 and IgG4. The constant region may have been modified in a preferred embodiment so that the antibody does not have effector functions. Hence, it will be appreciated that sequence variants of these constant region domains may also be used. For example IgG4 molecules in which the serine at position 241 has been changed to proline as described in Angal et al., 1993, Molecular Immunology, 1993, 30:105-108 may be used. Accordingly, in the embodiment, where the antibody is an IgG4 antibody, the antibody may include the mutation S241P. In another embodiment, an antibody of the invention may lack an Fc region.

An antibody of the invention may have, in one embodiment, a silenced Fc region. The term “silent”, “silenced”, or “silencing” as used herein refers to an antibody having a modified Fc region described herein that has decreased binding to an Fc gamma receptor (FcgR) relative to binding of an identical antibody comprising an unmodified Fc region to the FcgR (e.g., a decrease in binding to a FcgR by at least 70%, at least 80%, at least 90%, at least 95%, at least 98%, at least 99%, or 100% relative to binding of the identical antibody comprising an unmodified Fc region to the FcgR as measured by, e.g., BLI). In some embodiments, the Fc silenced antibody has no detectable binding to an FcgR. Binding of an antibody having a modified Fc region to an FcgR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). In another embodiment, an antibody of the present invention may have been modified to reduce or eliminate binding to the FcgR, but still allow activation of complement. In another embodiment, an antibody of the present invention may have a modified Fc region such that it does not activate cytokine release, but is still able to activate complement.

In one embodiment, the antibody heavy chain comprises a CH₁ domain and the antibody light chain comprises a CL domain, either kappa or lambda. In one embodiment, the antibody heavy chain comprises a CH₁ domain, a CH₂ domain and a CH₃ domain and the antibody light chain comprises a CL domain, either kappa or lambda.

The four human IgG isotypes bind the activating Fcγ receptors (FcγRI, FcγRIIa, FcγRIIc, FcγRIIIa), the inhibitory FcγRIIb receptor, and the first component of complement (C1q) with different affinities, yielding very different effector functions (Bruhns P. et al., 2009. Specificity and affinity of human Fcgamma receptors and their polymorphic variants for human IgG subclasses. Blood. 113(16):3716-25), see also Jeffrey B. Stavenhagen, et al. Cancer Research 2007 Sep. 15; 67(18):8882-90. In one embodiment, an antibody of the invention does not bind to Fc receptors. In another embodiment of the present invention, the antibody does bind to one or more type of Fc receptor.

Binding of IgG to the FcγRs or C1q depends on residues located in the hinge region and the CH₂ domain. Two regions of the CH₂ domain are critical for FcγRs and C1q binding, and have unique sequences in IgG2 and IgG4. Substitutions into human IgG1 of IgG2 residues at positions 233-236 and IgG4 residues at positions 327, 330 and 331 have been shown to greatly reduce ADCC and CDC (Armour K L. et al., 1999. Recombinant human IgG molecules lacking Fcgamma receptor I binding and monocyte triggering activities. Eur J Immunol. 29(8):2613-24 and Shields R L. et al., 2001. High resolution mapping of the binding site on human IgG1 for Fc gamma RI, Fc gamma RII, Fc gamma RIII, and FcRn and design of IgG1 variants with improved binding to the Fc gamma R. J Biol Chem. 276(9):6591-604). Furthermore, Idusogie et al. demonstrated that alanine substitution at different positions, including K322, significantly reduced complement activation (Idusogie E E. et al., 2000. Mapping of the C1q binding site on rituxan, a chimeric antibody with a human IgG1 Fc. J Immunol. 164(8):4178-84). Similarly, mutations in the CH₂ domain of murine IgG2A were shown to reduce the binding to FcγRI, and C1q (Steurer W. et al., 1995. Ex vivo coating of islet cell allografts with murine CTLA4/Fc promotes graft tolerance. J Immunol. 155(3):1165-74).

In one embodiment the Fc region employed is mutated, in particular a mutation described herein. In one embodiment the mutation is to remove binding and/or effector function. In one preferred embodiment the antibody of the invention has been mutated so that it does not bind Fc receptors. In another preferred embodiment, an antibody of the present invention does not comprise an Fc region and so does not display Fc effector activity for that reason. In one embodiment the Fc mutation is selected from the group comprising a mutation to remove or enhance binding of the Fc region to an Fc receptor, a mutation to increase or remove an effector function, a mutation to increase or decrease half-life of the antibody and a combination of the same. In a preferred embodiment, the modification eliminates or reduces binding to Fc receptors. In another preferred embodiment, the modification eliminates or reduces an Fc effector function. In another preferred embodiment, the modification reduces serum half-life. In another preferred embodiment, the constant region of the antibody comprises a modification or modifications that reduce or eliminate Fc receptor binding, and Fc effector function, as well as reducing serum half-life. In one embodiment, where reference is made to the impact of a modification it may be demonstrated by comparison to the equivalent antibody but lacking the modification.

In another embodiment of the present invention, an antibody may have heavy chain modifications that modify the ability to bind Protein A and in particular to eliminate Protein A binding. As discussed herein, such an approach may be preferably used to facilitate purification of bispecific antibodies. However, in other embodiments, any antibody of the invention may be modified, if it has an Fc region, to alter Protein A binding. For example, both heavy chains may include the modification. Alternatively, both heavy chains may lack the modification. In a preferred embodiment though, one has the modification and the other not.

Some antibodies that selectively bind FcRn at pH 6.0, but not pH 7.4, exhibit a higher half-life in a variety of animal models. Several mutations located at the interface between the CH₂ and CH₃ domains, such as T250Q/M428L (Hinton P R. et al., 2004. Engineered human IgG antibodies with longer serum half-lives in primates. J Biol Chem. 279(8):6213-6) and M252Y/S254T/T256E+H433K/N434F (Vaccaro C. et al., 2005. Engineering the Fc region of immunoglobulin G to modulate in vivo antibody levels. Nat Biotechnol. 23(10):1283-8), have been shown to increase the binding affinity to FcRn and the half-life of IgG1 in vivo. Hence, modifications may be present at M252/5254/T256+H44/N434 that alter serum half-life and in particular M252Y/S254T/T256E+H433K/N434F may be present. However, there is not always a direct relationship between increased FcRn binding and increased half-life (Datta-Mannan A. et al., 2007. Humanized IgG1 Variants with Differential Binding Properties to the Neonatal Fc Receptor: Relationship to Pharmacokinetics in Mice and Primates. Drug Metab. Dispos. 35: 86-94). In one embodiment, it is desired to increase half-life. In another embodiment, it may be actually desired to decrease serum half-life of the antibody and so modifications may be present that decrease serum half-life.

IgG4 subclass show reduced Fc receptor (FcγRIIIa) binding, antibodies of other IgG subclasses generally show strong binding. Reduced receptor binding in these other IgG subtypes can be effected by altering, for example replacing one or more amino acids selected from the group comprising Pro238, Aps265, Asp270, Asn270 (loss of Fc carbohydrate), Pro329, Leu234, Leu235, Gly236, Gly237, Ile253, Ser254, Lys288, Thr307, Gln311, Asn434 and His435. In one embodiment a molecule according to the present invention has an Fc of IgG subclass, for example IgG1, IgG2 or IgG3 wherein the Fc is mutated in one, two or all following positions 5228, L234 and/or D265. In one embodiment the mutations in the Fc region are independently selected from S228P, L234A, L235A, L235A, L235E and combinations thereof.

In one embodiment, an antibody of the present invention may comprise modifications that influence whether an antibody brings about cytokine release. In particular, the L234F and K274Q modifications are shown to reduce the ability of the antibody to bring about cytokine release. Hence, in one embodiment, an antibody of the present invention may comprise modifications at L234 and/or K274 that alter cytokine release and in particular the L234F and K274Q modifications. Further, the L234 residue may have an impact on platelet activation and that residue may be additionally or alternatively modified. In one embodiment of the invention, for example a L234 modification that alters platelet binding and in particular an L234F modification may be introduced. P331 is also shown to play a role in C1q binding, so in one embodiment P331 may be unmodified in order to retain complement activation. In another it may be modified to reduce or eliminate complement activation; for instance the heavy chains may comprise a P331S modification. In another embodiment, a P329 modification is present that reduces or eliminates complement binding, in particular a P329A modification. In another embodiment, the antibody may comprise one or more of the modifications at positions P329, P331, K332 and/or D265. In one preferred embodiment, an antibody may comprise modifications at P329A, P331S, K332A, and D265A to influence complement binding and in particular to reduce C1q binding.

It may be desired to either reduce or increase the effector function of an Fc region. In one preferred embodiment, it is desired to decrease such effector functions. In another, it is desired to optimise it. With antibodies that target cell-surface molecules, especially those on immune cells, abrogating effector functions is typically required. In other instances, particularly where the aim is to deplete cells, it may be desirable for Fc effector functions to have been eliminated or reduced to as low a level as possible. For instance, in a particularly preferred embodiment, an antibody of the present invention is able to induce cell death (preferably apoptosis) in target cells expressing CD45, but does not display Fc effector functions. Hence, in one preferred embodiment, an antibody of the invention lacks an active Fc region. For instance, the antibody may not physically have an Fc region or the antibody may comprise modifications that render the Fc region inactive. The latter may be, for instance, referred to as Fc silencing. In one embodiment, the Fc silencing may mean that an antibody of the invention is less able, or does not, bring about release of one or more cytokine which an antibody with an unmodified Fc region would usually trigger release of. In one preferred embodiment, an antibody of the invention is able to stimulate cell death (preferably apoptosis), but does not display Fc functions. Further examples of Fc functions include the stimulation of degranulation of Mast cells and again that function may be reduced or absent in an antibody of the invention. The degree in reduction of Fc function may be, for instance, at least 65%, and, for example, at least 75%. In one embodiment, the reduction is at least 80%. In another embodiment, the reduction is at least 90%. The reduction may be, for instance, at least 95%. In one preferred embodiment, the reduction is by at least 99%. In another embodiment, the reduction may be 100%, meaning that Fc function is completely eliminated in such instances.

Numerous mutations have been made in the CH₂ domain of human IgG1 and their effect on ADCC and CDC tested in vitro (Idusogie E E. et al., 2001. Engineered antibodies with increased activity to recruit complement. J Immunol. 166(4):2571-5). Notably, alanine substitution at position 333 was reported to increase both ADCC and CDC. Hence, in one embodiment a modification at position 333 may be present, and in particular one that alters ability to recruit complement. Lazar et al. described a triple mutant (S239D/I332E/A330L) with a higher affinity for FcγRIIIa and a lower affinity for FcγRIIb resulting in enhanced ADCC (Lazar G A. et al., 2006). Hence, modifications at S2394332/A330 may be present, particularly those that alter affinity for Fc receptors and in particular S239D/I332E/A330L. Engineered antibody Fc variants with enhanced effector function. PNAS 103(11): 4005-4010). The same mutations were used to generate an antibody with increased ADCC (Ryan M C. et al., 2007. Antibody targeting of B-cell maturation antigen on malignant plasma cells. Mol. Cancer Ther., 6: 3009-3018). Richards et al. studied a slightly different triple mutant (S239D/I332E/G236A) with improved FcγRIIIa affinity and FcγRIIa/FcγRIIb ratio that mediates enhanced phagocytosis of target cells by macrophages (Richards J O et al (2008) Optimization of antibody binding to Fcgamma RIIa enhances macrophage phagocytosis of tumor cells. Mol Cancer Ther. 7(8):2517-27). In one embodiment, S239D/I332E/G236A modifications may be therefore present.

Due to their lack of effector functions, IgG4 antibodies represent a suitable IgG subclass for receptor blocking. IgG4 molecules can exchange half-molecules in a dynamic process termed Fab-arm exchange. This phenomenon can occur between therapeutic antibodies and endogenous IgG4. In one preferred embodiment, an antibody of the present invention has a modification at S228 and in particular S228P. The S228P mutation has been shown to prevent this recombination process allowing the design of less unpredictable therapeutic IgG4 antibodies (Labrijn A F. et al., 2009. Therapeutic IgG4 antibodies engage in Fab-arm exchange with endogenous human IgG4 in vivo. Nat Biotechnol. 27(8):767-71). This technology may be employed to create bispecific antibody molecules. The modifications set out herein may, in a preferred embodiment, be employed in the context of IgG4.

WO 2008/145142 discloses examples of modifications and in particular modifications for IgG4 isotype antibodies that may be employed in the present invention. In one embodiment, the heavy chains of an antibody of the present invention may comprise a human IgG4 constant region having a substitution of the Arg residue at position 409, the Phe residue at position 405 and/or the Lys residue at position 370. For example, in one preferred embodiment the heavy chains of the antibody comprise a modification at position 409 and in particular one selected from the introduction of a Lys, Ala, Thr, Met, or Leu residue at that position. In one embodiment, the modification is the introduction of a Lys, Thr, Met, or Leu residue at position 409. In another embodiment, the modification may be the introduction of a Lys, Met or Leu residue at position 409. In one embodiment, the antibody does not comprise a Cys-Pro-Pro-Cys in the hinge region. In one embodiment, the antibody shows reduced ability to induce Fab arm exchange in vivo. In one embodiment, the hinge region of the antibody comprises a CXPC or CPXC sequence where X is any amino acid except proline. In one embodiment, an antibody of the invention may employ the ability of a particular antibody class, antibody isotype, or antibody allotype to display a particular property. Such natural diversity may be used to confer a particular property. For example, IgG1 has R409 whereas IgG4 has K409 at position 409 of the heavy chain which may naturally influence the ability of the antibody. A review of various naturally occurring sequence variations is provided in Jefferis et al (2009) mAbs, 1(4): 332-338, which is incorporated by reference in its entirety in particular in relation to the sequence variations discussed therein.

It will also be understood by one skilled in the art that antibodies may undergo a variety of post-translational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995). Accordingly, the C-terminal lysine of the antibody heavy chain may be absent.

In one embodiment, an antibody of the present invention may be an aglycosyl IgG, for example to bring about reduced Fc function and in particular a nearly Fc-null phenotype. In one embodiment, an antibody of the invention has a modification at N297 and in particular N297A. In one embodiment an antibody of the invention has modifications at F243 and/or F244, in particular ones that mean that the antibody is an aglycosyl IgG. In one embodiment, an antibody of the present invention may comprise the F243A and/or F244A heavy chain modifications. In another embodiment, one or more of F241, F243, V262 and V264 may be modified and particularly to amino acids that influence glycosylation. In one embodiment, an antibody of the present invention may have modifications at F241A, F243A, V262E and V264E. Such modifications are discussed in Yu et al (2013) 135(26): 9723-9732, which is incorporated by reference in its entirety, particularly in relation to the modifications discussed therein. Such modifications provide a way to modulate, for example, Fc receptor binding. A modification which influences the glycosylation of the antibody may be present. Further, an antibody of the invention may be produced in a cell type that influences glycosylation as a further approach for sugar engineering. In one embodiment, the fucosylation, sialylation, galactosylation, and/or mannosylation of an antibody of the present invention may be altered either by sequence modifications and/or via the type of cell used to produce the antibody.

In one embodiment, an antibody of the present invention has modifications at position 297 and/or 299. For example, in one embodiment, an antibody of the present invention comprises a N297A modification in its heavy chains, preferably N297Q or mutation of Ser or Thr at 299 to other residues. In one embodiment it has both those modifications.

In one embodiment, an antibody of the present invention may have modifications that favour the formation of an antibody of the invention over unwanted species. For example, in one embodiment the production of an antibody of the invention may involve two different antigen sites, in particular two different paratopes, being on different units and associated. Hence, it may be desirable to form heterodimers which include both paratopes in preference to homodimers which only include one of the paratopes. An example of an approach that favours heterodimer formation is employing heavy chain modifications that favour two different heavy chains, rather than two of the same heavy chains associating. In one embodiment one (or at least one) of the binding partners is incapable of forming a homodimer, for example an amino acid sequence of the binding partner is mutated to eliminate or minimise the formation of homodimers. Examples of such modifications include so called “knobs-into-holes” modifications. Possible knobs-into-holes modifications are set out, for instance, in Merchant et al (1998) Nature Biotechnology 16(7): 677-681 and Carter et al (2001) J Immunol Methods, 248(1-2): 7-15, which are both incorporated by reference in particular in relation to the knobs-into-holes modifications discussed therein. Charge modifications may be alternatively or additionally employed to favour formation of heterodimers over homodimers, for example such modifications may be present in the heavy chains. In another embodiment, charge modifications are used to bring about pairing of a particular light chain with a particular heavy chain.

In one embodiment, such approaches for favouring heterodimer formation are used in combination with a common light chain approach. In another embodiment, it may be that rather favouring the formation of heterodimer over homodimers, modifications are present that mean the heterodimers can be separated from the homodimers more easily, for instance by chromatography. Again, such an approach may be, in some embodiments, employed with a common light chain approach. In another embodiment, the portions of the antibody carrying a particular paratope against CD45 are only able to associate with those portions of the antibody which comprise the different paratope of the antibody.

In one embodiment both of the binding partners are incapable of forming a homodimer, for example one of the binding partners is a peptide and the other binding partner is a V_{HH} specific to said peptide. In one embodiment a scFv employed in the molecules of the present invention is incapable of forming a homodimer.

Incapable of forming homodimers as employed herein, refers to a low or zero propensity to form homodimers. Low as employed herein refers to 5% or less, such as 4, 3, 2, 1, 0.5% or less aggregate.

In another embodiment, an antibody of the present invention may have a modified hinge region and/or CH1 region. Alternatively, the isotype employed may be chosen as it has a particular hinge regions. As described in White et al (2015) Cancer Cell 27(1): 138-148, the IgG2 CH1 and hinge regions confer particular properties, particularly in relation to disulphide bridges between the heavy and light chains. The use of modifications to favour flexibility in the hinge region or reduced flexibility may also be employed, for example, in an antibody of the present invention. Approaches to alter hinge region flexibility are disclosed in Liu et al (2019) Nature Communications 10: 4206. White et al (2015) and Liu et al (2019) are incorporated by reference in their entirety, particularly in relation to the modifications discussed. In one embodiment, a heavy chain of an antibody of the present invention has an IgG2 CH1 and/or hinge region and in another embodiment both heavy chains do so. In one embodiment, the antibody employed is an h2 antibody. In a particularly preferred embodiment, the antibody employed may be an IgG2 or IgG4 antibody with a hinge or CH1 modification, in particular one with a modified hinge region, for example one engineered to alter disulphide bond formation. In another embodiment, an IgG2 or IgG4 isotype antibody is employed, as the hinge regions of those isotypes show less flexibility than an IgG3 isotype antibody. In one embodiment, an IgG4 isotype antibody is employed in a form that may be able to bring about CD32 cross-linking.

In another embodiment, the antibody shows the best ability sterically to bring about cross-linking of CD45 molecules.

### Bispecific Antibodies

In one preferred embodiment, a binding molecule, and in particular an antibody of the present invention, is bispecific. Hence, in a preferred embodiment, a bispecific antibody is employed in the present invention and in a particularly preferred embodiment a bispecific antibody with two different specificities for CD45. A variety of bispecific antibody formats are available for favouring formation, or purification, of bispecific antibodies over monospecific antibodies when the different heavy and light chains for the specificities are expressed together, and these may be employed in the present invention.

For example, shape or charge modifications may be present in the heavy chain for one or both specificities that favour heterodimer formation over homodimer formation. Examples of such modifications include knob-in-hole heavy chain modifications that mean the two different heavy chains for the different specificities are more likely to interact and hence favour the formation of heterodimers. The strand-exchange engineered domains (SEEDbody) approach may also be used to favour heterodimer formation.

Heavy chain modifications may also be employed so that one heavy chain has a different affinity for a binding agent compared to the other. For example, the two different heavy chains may have different affinity for Protein A. In one embodiment, one heavy chain has a modification that eliminates Protein A binding or is of an isotype that does not bind Protein A, whilst the other heavy chain does still bind Protein A. Whilst such an approach does not alter the proportion of heterodimer formed, it does allow the purification of the heterodimeric antibody from either of the homodimeric antibodies based on Protein A affinity. An antibody of the present invention may have modifications at positions 95 and 96 of one of the heavy chains that influence Protein A binding. Examples of such modifications that may be employed include employing a H95R modification for one heavy chain or the H95R and Y96F modifications both in the IMGT exon numbering system. Those modifications are the H435R modification and H435R and Y436F modification in the EU numbering system. In one embodiment, an antibody of the present invention may also have modifications at D16, L18, N44, K52, V57 and V82. In one embodiment, such modifications are present in the heavy chain as well as one or more of the D16E, L18M, N44S, K52N, V57M and V82l modifications in the IMGT numbering system. In one embodiment, such modifications are employed where the IgG is IgG1, IgG2 or IgG4. In a particularly preferred embodiment, they are employed for one of the two heavy chains where both heavy chains are the IgG4 isotype. The approach of such modifications to influence Protein A binding is described in, for instance, US 2010/0331527 A1, which is incorporated by reference in its entirety and in particular in relation to the modifications it discloses that relate to Protein A binding.

In a further embodiment, the isotype of the heavy chains employed may be chosen based on their ability to bind Protein A. For example, in humans IgG1, IgG2, and IgG4 in their wild type form all bind Protein A, whereas wild type human IgG3 does not. In a particularly preferred embodiment, both heavy chains are IgG4, but one has modification(s) to reduce or eliminate Protein A binding. That means the heterodimeric form of the antibody will be able to be separated from the unwanted homodimeric forms more readily based on Protein A affinity.

In one embodiment, modifications to promote heterodimer formation may be combined with those that allow purification of the heterodimer. In one embodiment, the modifications may be at positions F405 and K409. For example, one example of a pair of modifications that may be introduced into the two heavy chains to favour heterodimer formation are F405L and K409R. Those modifications may be employed on their own or in combination with heavy chain modifications allowing preferential purification of the heterodimer. In one embodiment, one heavy chain has modifications at positions 405, 409, 435, and 436 and the other heavy chain at position 409. In one embodiment, one heavy chain has the F405L modification with the other having the K409R, H435R and Y436F modifications. In another embodiment, one heavy chain has the F405L, H435R and Y436F modification and the other heavy chain has the K409R modification. Examples of such approaches are described in Steinhardt et al (2020) Pharmaceutics, 12, 3, which is incorporated by reference in its entirety, in particular in relation to the bispecific antibody formats and heavy chain modifications described. In another embodiment, approaches concerned with the light chain may be employed and in particular in addition to the approaches for the heavy chain discussed above. For example, for one light chain portions of the light and heavy chain it is desired to pair with may be swapped with each other to favour formation of that light chain heavy pairing, whilst the heavy chain for the other specificity and light chain are unmodified. In one embodiment, the Roche Cross-Mab approach is therefore applied. In another embodiment a common light chain may be employed so that the same light chain is employed for both specificities. Various bispecific antibody formats are reviewed in Spiess et al (2015) Molecular Immunology 67: 95-106 and may be employed in the present invention, including in particular those shown in FIG. 1 of that reference. Spiess et al (2015) is incorporated by reference, including in particular for the types of antibody format shown in FIG. 1 of that reference.

### Antibody Generation and Screening

In one embodiment, the antibodies of the present invention or antibody/fragment components thereof are processed to provide improved affinity for a target antigen or antigens and in particular for CD45. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al J. Mol. Biol., 250, 359-368, 1996), DNA shuffling (Patten et al Curr. Opin. Biotechnol., 8, 724-733, 1997), phage display (Thompson et al., J. Mol. Biol., 256, 77-88, 1996) and sexual PCR (Crameri et al Nature, 391, 288-291, 1998). Vaughan et al (supra) discusses these methods of affinity maturation. Binding domains for use in the present invention may be generated by any suitable method known in the art, for example CDRs may be taken from non-human antibodies including commercially available antibodies and grafted into human frameworks or alternatively chimeric antibodies can be prepared with non-human variable regions and human constant regions etc.

Examples of CD45 antibodies are known in the art and a paratope from such antibody may be employed in an antibody of the present invention which has more than one specificity for CD45 or screened for suitability using the methods described herein, and subsequently modified if necessary, for example humanised, using the methods described herein. Therapeutic anti-CD45 antibodies have been described in the art, for example anti-CD45 antibodies disclosed in US2011/0076270. Examples of CD45 antibodies include rat monoclonal YTH54, YTH25.4, mouse monoclonal from Miltenyi clone 5B1 and clone 30F11, rat monoclonal YAML568, from BD Bioscience mouse monoclonal clone 2D1 catalog No. 347460, from Novus mouse monoclonal antibody 5D3A3 catalog No. NBP2-37293, mouse monoclonal HI30 catalog No. NBP1-79127, mouse monoclonal 4A8A4C7A2 catalog No. NBP1-47428, mouse monoclonal 2B11 catalog No. NBP2-32934, rat monoclonal YTH24.5 catalog No. NB100-63828, rabbit monoclonal Y321 catalog No. NB110-55701, mouse monoclonal PD7/26/16 catalog No. NB120-875, from Santa Cruz mouse monoclonal from clone B8 catalog No. sc-28369, mouse monoclonal from clone F10-89-4 catalog No. sc-52490, rabbit monoclonal from clone H-230 catalog No. sc-25590, goat monoclonal from clone N-19 catalog No. sc-1123, mouse monoclonal from clone OX1 catalog No. sc-53045, rat monoclonal (T29/33) catalog No sc-18901, rat monoclonal (YAML 501.4) catalog No. sc65344, rat monoclonal (YTH80.103) catalog No sc-59071, mouse monoclonal (35105) catalog No. sc-53201, mouse monoclonal (35-Z6) catalog No. sc-1178, mouse monoclonal (158-4D3) catalog No. sc-52386, mouse monoclonal to CD45RO (UCH-L1) catalog No. sc-1183, mouse monoclonal to CD45RO (2Q1392) catalog No. sc-70712. CD45 antibodies are also disclosed in WO2005/026210, WO02/072832 and WO2003/048327 incorporated herein by reference. Such commercially available antibodies may be useful tools in the discovery of therapeutic antibodies. In one particularly preferred embodiment the antibody of the invention is a human antibody or is one that has been humanised. Hence, commercial antibodies may be humanised in one embodiment. The present application though sets out examples of particular preferred antibodies, as well as methods for identifying further antibodies.

The skilled person may generate antibodies for use in the antibodies of the invention using any suitable method known in the art. Antigen polypeptides, for use in generating antibodies for example for use to immunize a host or for use in panning, such as in phage display, may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The antigen polypeptide may in some instances be part of a larger protein such as a fusion protein for example fused to an affinity tag or similar. In one embodiment, the host may be immunised with a cell transfected with CD45, for instance expressing CD45 on its surface.

Antibodies generated against an antigen polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable. Monoclonal antibodies may be prepared by any method known in the art such as the hybridoma technique (Kohler & Milstein, 1975, Nature, 256:495-497), the trioma technique, the human B-cell hybridoma technique (Kozbor et al 1983, Immunology Today, 4:72) and the EBV-hybridoma technique (Cole et al Monoclonal Antibodies and Cancer Therapy, pp 77-96, Alan R Liss, Inc., 1985).

Antibodies may also be generated using single lymphocyte antibody methods by cloning and expressing immunoglobulin variable region cDNAs generated from single lymphocytes selected for the production of specific antibodies by, for example, the methods described by Babcook, J. et al 1996, Proc. Natl. Acad. Sci. USA 93(15):7843-78481; WO92/02551; WO2004/051268 and WO2004/106377. The antibodies for use in the present invention can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO90/02809; WO91/10737; WO92/01047; WO92/18619; WO93/11236; WO95/15982; WO95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; 5,969,108, and WO20011/30305. In one preferred embodiment, an antibody of the present invention has at least two different paratopes specific for CD45 and it may be that antibodies recognising one paratope of CD45 are first raised and then, for instance, two of those antibodies are used to generate an antibody of the present invention able to bind at least two different paratopes of CD45. It may be, for instance, that multiple antibodies against CD45 are raised using the methods discussed herein and then screened for desirable properties, such as binding affinities. Then the best candidates may be used to generate an antibody of the present invention.

In one example an antibody of the present invention is fully human, in particular one or more of the variable domains are fully human. Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP 0438474 and EP0463151. Monoparatopic antibodies may be first raised and then used to generate an antibody of the invention that comprises at least two different paratopes against CD45.

In one example, the antigen-binding sites, and in particular the variable regions, of the antibodies according to the invention are humanised. Humanised (which include CDR-grafted antibodies) as employed herein refers to molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). In a preferred embodiment though, the whole CDR or CDRs is/are transplanted. Humanised antibodies may optionally further comprise one or more framework residues derived from the non-human species from which the CDRs were derived. As used herein, the term “humanised antibody molecule” refers to an antibody molecule wherein the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment, rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment, only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the humanised antibody according to the present invention has a variable domain comprising human acceptor framework regions as well as one or more of the CDRs provided herein. Examples of human frameworks which can be used in the present invention are KOL, NEWM, REI, EU, TUR, TEI, LAY and POM (Kabat et al supra). For example, KOL and NEWM can be used for the heavy chain, REI can be used for the light chain and EU, LAY and POM can be used for both the heavy chain and the light chain. Alternatively, human germline sequences may be used; these are available at: http://www2.mrc-lmb.cam.ac.uk/vbase/list2.php.

In a humanised antibody molecule of the present invention, the acceptor heavy and light chains do not necessarily need to be derived from the same antibody and may, if desired, comprise composite chains having framework regions derived from different chains. The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al 1998, Nature, 332, 323-324). Such changes should be kept to the minimum necessary to recover the affinity of the donor antibody. A protocol for selecting residues in the acceptor framework regions which may need to be changed is set forth in WO 91/09967. Derivatives of frameworks may have 1, 2, 3, 4, 5, 6, 7, 8, 9, 10 or more amino acids replaced with an alternative amino acid, for example with a donor residue. Donor residues are residues from the donor antibody, i.e. the antibody from which the CDRs were originally derived, in particular the residue in a corresponding location from the donor sequence is adopted. Donor residues may be replaced by a suitable residue derived from a human receptor framework (acceptor residues).

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated. The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition. The CDRs of the light chain variable domain are located at residues 24-34 (CDR-L1), residues 50-56 (CDR-L2) and residues 89-97 (CDR-L3) according to the Kabat numbering system.

In one embodiment the invention extends to an antibody sequence disclosed herein, in particular humanised sequences disclosed herein.

In one example the binding domains are humanised.

In one example one or more CDRs provided herein may be modified to remove undesirable residues or sites, such as cysteine residues or aspartic acid (D) isomerisation sites or asparagine (N) deamidation sites. In one example an Asparagine deamidation site may be removed from one or more CDRs by mutating the asparagine residue (N) and/or a neighbouring residue to any other suitable amino acid. In one example an asparagine deamidation site such as NG or NS may be mutated, for example to NA or NT.

In one example an Aspartic acid isomerisation site may be removed from one or more CDRs by mutating the aspartic acid residue (D) and/or a neighbouring residue to any other suitable amino acid. In one example an aspartic acid isomerisation site such as DG or DS may be mutated, for example to EG, DA or DT.

For example one or more cysteine residues in any one of the CDRs may be substituted with another amino acid, such as serine.

In one example an N-glycosylation site such as NLS may be removed by mutating the asparagine residue (N) to any other suitable amino acid, for example to SLS or QLS. In one example an N-glycosylation site such as NLS may be removed by mutating the serine residue (S) to any other residue with the exception of threonine (T).

The skilled person is able to test variants of CDRs or humanised sequences in any suitable assay such as those described herein to confirm activity is maintained.

Specific binding to antigen may be tested using any suitable assay including for example ELISA or surface plasmon resonance methods such as BIAcore where binding to antigen (CD45) may be measured. Such assays may use isolated natural or recombinant CD45 or a suitable fusion protein/polypeptide. In one example, binding is measured using recombinant CD45 (SEQ ID NO: 41 or amino acids 23-1304 of SEQ ID NO:41) by, for example, surface plasmon resonance, such as BIAcore. Alternatively the proteins may be expressed on a cell, such as a HEK cell and affinity measured employing a flow cytometry based affinity determination. In one embodiment, where it is desired to determine the properties of one antigen-binding site, in particular paratope, on its own, an antibody is generated with just that paratope. For example, the same format antibody as an antibody of the invention with two different specificities is generated, but with just one of the specificities for CD45 present. In one embodiment, antibodies for each of the paratopes against CD45 from an antibody of the present invention with at least two paratopes may be generated, for instance to allow the affinity of each paratope to be determined or to determine whether or not the paratopes display cross-blocking against each other. In one embodiment the ability to bind the extracellular region of CD45 is measured, for instance using the protein of SEQ ID NO: 113. In one embodiment, monovalent antibodies, such as ScFv are generated to perform the comparison.

Antibodies which include a paratope that cross-blocks the binding of a paratope of an antibody molecule according to the present invention may be similarly useful in binding CD45 and therefore similarly useful antibodies, for example, in the antibodies of the present invention. Hence, employing such cross-blocking or competition assays may be a useful way of identifying and generating antibodies of the present invention. In one embodiment, an individual paratope from an antibody of the invention may be used to generate a bivalent antibody where both antigen-binding sites comprise that paratope, with that bivalent antibody then used in cross-blocking assays.

In another embodiment, an antibody which is able to cross-block at least one of the paratopes of one of the antibodies disclosed herein may be employed in generating an antibody molecule of the present invention. In one embodiment, an antibody of the invention may comprise one of the paratopes set out herein or one that is able to cross-block or compete with it. Accordingly, the present invention also provides an antibody molecule comprising a binding domain specific to the antigen CD45, wherein the binding domain for CD45 cross-blocks the binding of at least one of the paratopes specific for CD45 of any one of the antibody molecules described herein above to CD45 and/or is cross-blocked from binding CD45 by any one of those paratopes. Overall, typically though the different paratopes specific for CD45 in an antibody of the invention with different paratopes for CD45 will not cross-block or compete with each other for binding to CD45 or will not do so significantly. For instance, less than 30%, preferably less than 25%, more preferably less than 10% cross-blocking will be seen. In one embodiment, less than 5% and in particular less than 1% cross-blocking will be seen. In one embodiment, a cross-blocking paratope is wanted as a way to identify further paratopes specific for CD45 to be employed in an antibody of the invention, for instance to replace an existing paratope that cross-blocks. In another embodiment the cross-blocking antibody binds to an epitope which borders and/or overlaps with the epitope bound by the paratope specific for CD45 of an antibody described herein above. In another embodiment, the cross-blocking neutralising antibody binds to an epitope which borders and/or overlaps with the epitope bound by the paratope against CD45 an antibody described herein above. Cross-blocking assays may be also employed to checked that the at least two different paratopes against CD45 of an antibody of the present invention bind to different epitopes of CD45 and so do not cross-block the binding of each other. In one embodiment, two antibodies each comprising just one of the paratopes against CD45 may be generated and the ability of each to cross-block the other measured. Whilst antibodies and/or specificities for CD45 should typically not cross-block or compete with other, they should be able to both still bind CD45 at the same time.

Cross-blocking antibodies can be identified using any suitable method in the art, for example by using competition ELISA or BIAcore assays where binding of the cross blocking antibody to antigen (CD45) prevents the binding of an antibody of the present invention or vice versa. Such cross blocking assays may use isolated natural or recombinant CD45 or a suitable fusion protein/polypeptide. In one example binding and cross-blocking is measured using recombinant CD45 (SEQ ID NO: 41), for example cross-blocking by any one of those antibodies is by 80% or greater, for example by 85% greater, such as 90% or greater, in particular by 95% or greater. In one embodiment, a cross-blocking assay may be performed using an antibody with just one of the paratopes specific for CD45 from an antibody of the present invention.

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656,).

The present invention also extends to novel polypeptide sequences disclosed herein and sequences at least 80% similar or identical thereto, for example 85% or greater, such 90% or greater, in particular 95%, 96%, 97%, 98% or 99% or greater similarity or identity. In one embodiment a sequence may have at least 99% sequence identity to at least one of the specific sequences provided herein. “Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).
   Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656,).

It will be appreciated that this aspect of the invention also extends to variants of these anti-CD45 antibodies including humanised versions and modified versions, including those in which amino acids have been mutated in the CDRs to remove one or more isomerisation, deamidation, glycosylation site or cysteine residue as described herein above. In one embodiment, one or both of the paratopes against CD45 may be from already known antibodies, but which have not been employed in a biparatopic antibody format.

Preferred Antibodies with FALA and Knob-In-Hole Modifications

In one particularly preferred embodiment of the invention, the antibody employed comprises heavy chains with FALA modifications. In particular, FALA modifications alter Fc receptor binding. In a further preferred embodiment, the antibody comprises a modification in the hinge region of the antibody and in particular a modification at position 228, preferably 228P. In one embodiment, an antibody has a heavy chain comprising modifications at position 228, 234, and 235. In a particularly preferred embodiment, the heavy chains of an antibody of the present invention will comprise S228P, F234A, and L235A FALA modifications. In an especially preferred embodiment of the present invention the antibody provided will be an IgG4(P) isotype antibody and comprise such modifications.

In another particularly preferred embodiment, an antibody of the present invention will comprise so called “knob-in-hole” modifications. In one embodiment, one heavy chain of the antibody comprises a modification at T355 and the other at T366, 368, and 407 and in particular to make complementary shapes in the two different heavy chains that mean they preferentially pair, rather than two identical heavy chains pairing. In particular, the heavy chain for one specificity may have a T355W “knob” modification, whilst the other has T366S, L368A, Y407V “hole” modifications. In a particularly preferred embodiment, the antibody of the present invention is an IgG4 isotype antibody and has such modifications.

In another especially preferred embodiment of the present invention, the FALA, hinge, and “knob-in-hole” modifications are combined. In a preferred embodiment, they are combined in the context of an IgG4 isotype antibody. In one embodiment, one heavy chain of the antibody has modifications at positions 228, 234, 235 and 355. In another embodiment, one heavy chain comprises modifications at positions 228, 234, 235, 366, 368 and 407. For example, in one embodiment one heavy chain has S228P, F234A, L235A, T355W modifications (so both FALA and a “knob” modification) and preferably the other has S228P, F234A, L235A, T366S, L368A, Y407V modifications (so both FALA and “hole” modifications).

In an especially preferred embodiment, an antibody of the present invention is a FALA IgG4(P) antibody. In a further especially preferred embodiment, it is a FALA knobs-in-holes IgG4(P) antibody.

In another embodiment, the above formats may be combined with other formats/modifications discussed herein. For example, they may also include the modifications discussed herein to remove Protein A binding at positions 95 and 96. In a further embodiment, they may include a common light chain and may also comprise the Protein A binding modification as well.

### Further Preferred Antibody Formats Including BYbe and TrYbe

In one aspect, there is provided an antibody molecule comprising or consisting of:
a) a polypeptide chain of formula (VII):

V_{H}-CH₁—W—(V₁)ₚ;
b) a polypeptide chain of formula (VIII):

V_{L}-C_{L}—Z—(V₂)_{q};
wherein:
V_{H} represents a heavy chain variable domain;
CH₁ represents a domain of a heavy chain constant region, for example domain 1 thereof;
W represents a bond or linker, for example an amino acid linker, except if p or q is zero in which case they will also be zero;
Z represents a bond or linker, for example an amino acid linker;
V₁ represents a dab, scFv, dsscFv or dsFv;
V_{L} represents a variable domain, for example a light chain variable domain;
C_{L} represents a domain from a constant region, for example a light chain constant region domain, such as Ckappa;
V₂ represents a dab, scFv, dsscFv or dsFv;
p is 0 or 1;
q is 0 or 1; and
   when p is 1 q is 0 or 1 and when q is 1 p is 0 or 1 i.e. p and q do not both represent 0,
where at least two of the antigen binding sites of the antibody are different paratopes against CD45, each recognising different epitopes against CD45.

In one example the binding domains specific for CD45 are selected from at least two of V₁, V₂ or V_{H}/V_{L}.

In one embodiment q is 0 and p is 1.

In one embodiment q is 1 and p is 1.

In one embodiment V₁ is a dab and V₂ is a dab and together they form a single binding domain of a co-operative pair of variable regions, such as a cognate V_{H}/V_{L} pair, which are optionally linked by a disulphide bond.

In one embodiment V_{H} and V_{L} are specific to CD45.

In one embodiment the V₁ is specific to CD45.

In one embodiment the V₂ is specific to CD45.

In one embodiment the V₁ and V₂ together (e.g. as binding domain) are specific to CD45 and V_{H} and V_{L} are specific to CD45.

In one embodiment the V₁ is specific to CD45.

In one embodiment the V₂ is specific to, CD45.

In one embodiment the V₁ and V₂ together (e.g. as one binding domain) are specific to CD45 and V_{H} and V_{L} are specific to CD45.

In one embodiment the V₁ is specific to CD45, V₂ is specific to CD45 and V_{H} and V_{L} are specific to CD45.

V₁, V₂, V_{H} and V_{L} in the constructs above may each represent a binding domain and incorporate any of the sequences provided herein.

W and Z may represent any suitable linker, for example W and Z may be independently SGGGGSGGGGS (SEQ ID NO: 67) or SGGGGTGGGGS (SEQ ID NO:114).

In one embodiment, when V₁ and/or V₂ are a dab, dsFv or a dsscFv, the disulfide bond between the variable domains V_{H} and V_{L} of V₁ and/or V₂ is formed between positions V_{H}44 and V_{L}100.

In a preferred embodiment of the invention, an antibody of the invention is in the BYbe antibody format. A BYbe format antibody comprises a Fab linked to only one scFv or dsscFv, as described for example in WO 2013/068571, and Dave et al, (2016)Mabs, 8(7): 1319-1335. Hence, for example in one preferred embodiment in the formula given above one of (V1)p and (V2)q will be a ScFv or a dsscFv and the other will be nothing, so that the BYbe format antibody comprises a Fab and only one scFv or dsscFv. For whichever of (V1)p and (V2)q is zero the corresponding W or Z will also be nothing and the other will be a bond or a linker. Preferably the BYbe format antibody comprises a Fab and dsscFv. In such BYbe format antibodies the two antigen-binding sites may be preferably both specific for CD45, with the two corresponding to the two different paratopes for different epitopes of CD45.

In a further especially preferred embodiment of the invention the antibody is in the TrYbe format. A TrYbe format comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibody fragments are described in International Patent Application Publication No WO 2015/197772, which is hereby incorporated by reference in its entirety and particularly with respect to the structure and discussion of antibody fragments. In respect of the formula given above, for a TrYbe antibody, p and q will both be one, with V1 and V2 being independently selected from a ScFv and dsscFv. In a preferred embodiment, V1 and V2 will both by a ScFv. In another embodiment, V1 and V2 will both be a dsscFv. In another embodiment one of V1 and V2 will be a ScFv and the other a dsscFv. At least two of the antigen-binding sites of the TrYbe will be specific for CD45, with the antibody comprising two different paratopes each specific for a different epitope of CD45. In one particularly preferred embodiment, the third antigen-binding site will be specific for albumin and in particular one of V1 and V2 will be specific for albumin. For example, VH/VL may be specific for CD45 (e.g. for a first epitope of CD45), one of V1 and V2 may be specific for CD45 (e.g. for a second epitope of CD45), and the other of V1 and V2 may be specific for albumin.

In one preferred embodiment, an antibody of the invention will comprise at least one paratope specific for albumin. In one embodiment, the antibody will be a TrYbe format antibody comprising the two paratopes specific for a different epitope of CD45 and also a third paratope specific for albumin. Examples of albumin binding antibody sequences which may be used to bind albumin include those disclosed in WO 2017/191062 the entirety of which is incorporated by reference, particularly so far as it relates to albumin binding sequences. Hence, an antibody of the invention may comprise a paratope from one of the albumin specific antibodies in WO 2017191062.

In an alternative embodiment, an antibody as discussed above has only one specificity for CD45, rather than at least two different ones. For example, one of the antigen binding sites of the antibody may be specific for CD45. In another embodiment, two of the antigen binding sites are specific for CD45, but have the same specificity. In a further embodiment, all three antigen-binding sites of an antibody set out above have the same specificity for CD45. In another embodiment, two of the antigen-binding sites have the same specificity for CD45 and the third is specific for serum albumin. In a preferred embodiment, such antibodies are used in mixtures of antibodies of the present invention.

### Disulphide Bridges

Where one or more pairs of variable regions in the antibody of the present invention comprise a disulphide bond between VH and VL this may be in any suitable position such as between two of the residues listed below (unless the context indicates otherwise Kabat numbering is employed in the list below). Wherever reference is made to Kabat numbering the relevant reference is Kabat et al., 1987, in Sequences of Proteins of Immunological Interest, US Department of Health and Human Services, NIH, USA.

In one embodiment, when V1 and/or V2 are a dsFv or a dsscFv in the formulae discussed above, the disulfide bond between the variable domains VH and VL of VI and/or V2 is between two of the residues listed below (unless the context indicates otherwise Kabat numbering is employed in the list below). Wherever reference is made to Kabat numbering the relevant reference is Kabat et al., 1987, in Sequences of Proteins of Immunological Interest, US Department of Health and Human Services, NIH, USA.

In one embodiment the disulfide bond is in a position selected from the group comprising:
V_{H}37+V_{L}95C see for example Protein Science 6, 781-788 Zhu et al (1997);
V_{H}44+V_{L}100 see for example; Biochemistry 33 5451-5459 Reiter et al (1994); or Journal of Biological Chemistry Vol. 269 No. 28 pp. 18327-18331 Reiter et al (1994); or Protein Engineering, vol. 10 no. 12 pp. 1453-1459 Rajagopal et al (1997);
V_{H}44+V_{L}105 see for example J Biochem. 118, 825-831 Luo et al (1995);
V_{H}45+V_{L}87 see for example Protein Science 6, 781-788 Zhu et al (1997);
V_{H}55+V_{L}101 see for example FEBS Letters 377 135-139 Young et al (1995);
V_{H}100+V_{L}50 see for example Biochemistry 29 1362-1367 Glockshuber et al (1990);
V_{H}100b+V_{L}49;
V_{H}98+V_{L}46 see for example Protein Science 6, 781-788 Zhu et al (1997);
V_{H}101+V_{L}46;
V_{H}105+V_{L}43 see for example; Proc. Natl. Acad. Sci. USA Vol. 90 pp. 7538-7542 Brinkmann et al (1993); or Proteins 19, 35-47 Jung et al (1994), and
V_{H}106+V_{L}57 see for example FEBS Letters 377 135-139 Young et al (1995) and a position corresponding thereto in variable region pair located in the molecule.

In one embodiment, the disulphide bond is formed between positions V_{H}44 and V_{L}100.

The amino acid pairs listed above are in the positions conducive to replacement by cysteines such that disulfide bonds can be formed. Cysteines can be engineered into these desired positions by known techniques. In one embodiment therefore an engineered cysteine according to the present disclosure refers to where the naturally occurring residue at a given amino acid position has been replaced with a cysteine residue.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, N Y, 1989; Ausbel et al., Current Protocols in Molecular Biology, Greene Publishing & Wiley-Interscience, N Y, 1993). Site-directed mutagenesis kits are commercially available, e.g. QuikChange® Site-Directed Mutagenesis kit (Stratagen, La Jolla, CA). Cassette mutagenesis can be performed based on Wells et ah, 1985, Gene, 34:315-323. Alternatively, mutants can be made by total gene synthesis by annealing, ligation and PCR amplification and cloning of overlapping oligonucleotides.

WO 2015/197772 sets out in detail preferred locations for disulphide bridges in relation to BYbe and TrYbe format antibodies. WO 2015/197772 is incorporated by reference in its entirety, particularly in relation to the locations of the disulphide bridges.

As discussed herein, alteration of the ability of residues in the hinge regions of antibodies is one potential way to influence binding to CD45 and may be employed in the present invention.

### Linkers

The teaching herein of linkers in one context can equally be applied to linkers in different contexts where a linker is employed, such as in any binding molecule, an in particular antibody, of the present invention, particularly those that involve linking entities which each have different antigen-binding sites on them. In one embodiment, a linker may be employed to join together constituent parts of a binding molecule, in particular of an antibody of the invention. For example, in one embodiment a linker may be used to join a constituent part of a binding molecule, and in particular an antibody to one part of a heterodimeric tether, for example a Fab or ScFv may be joined to one of the two units of the heterodimeric tether via a linker.

In one embodiment, the linker employed in a molecule of the invention is an amino acid linker 50 residues or less in length, for example selected from a sequence shown in sequence 149 to 214.

TABLE 1
Hinge linker sequences

| SEQ ID NO: | SEQUENCE |
|---|---|
| 42 | DKTHTCAA |
| 43 | DKTHTCPPCPA |
| 44 | DKTHTCPPCPATCPPCPA |
| 45 | DKTHTCPPCPATCPPCPATCPPCPA |
| 46 | DKTHTCPPCPAGKPTLYNSLVMSDTAGTCY |
| 47 | DKTHTCPPCPAGKPTHVNVSVVMAEVDGTCY |
| 48 | DKTHTCCVECPPCPA |
| 49 | DKTHTCPRCPEPKSCDTPPPCPRCPA |
| 50 | DKTHTCPSCPA |

TABLE 2
Flexible linker sequences

| SEQ ID NO: | SEQUENCE |
|---|---|
| 51 | SGGGGSE |
| 52 | DKTHTS |
| 53 | (S)GGGGS |
| 54 | (S)GGGGSGGGGS |
| 55 | (S)GGGGSGGGGSGGGGS |
| 56 | (S)GGGGSGGGGSGGGGSGGGGS |
| 57 | (S)GGGGSGGGGSGGGGSGGGGSGGGGS |
| 58 | AAAGSG-GASAS |
| 59 | AAAGSG-XGGGS-GASAS |
| 60 | AAAGSG-XGGGSXGGGS-GASAS |
| 61 | AAAGSG-XGGGSXGGGSXGGGS-GASAS |
| 62 | AAAGSG-XGGGSXGGGSXGGGSXGGGS-GASAS |
| 63 | AAAGSG-XS-GASAS |
| 64 | PGGNRGTTTTRRPATTTGSSPGPTQSHY |
| 65 | ATTTGSSPGPT |
| 66 | ATTTGS |
| (no SEQ ID NO | GS |
| owing to | |
| length) | |
| 68 | EPSGPISTINSPPSKESHKSP |
| 69 | GTVAAPSVFIFPPSD |
| 70 | GGGGIAPSMVGGGGS |
| 71 | GGGGKVEGAGGGGGS |
| 72 | GGGGSMKSHDGGGGS |
| 73 | GGGGNLITIVGGGGS |
| 74 | GGGGVVPSLPGGGGS |
| 75 | GGEKSIPGGGGS |
| 76 | RPLSYRPPFPFGFPSVRP |
| 77 | YPRSIYIRRRHPSPSLTT |
| 78 | TPSHLSHILPSFGLPTFN |
| 79 | RPVSPFTFPRLSNSWLPA |
| 80 | SPAAHFPRSIPRPGPIRT |
| 81 | APGPSAPSHRSLPSRAFG |
| 82 | PRNSIHFLHPLLVAPLGA |
| 83 | MPSLSGVLQVRYLSPPDL |
| 84 | SPQYPSPLTLTLPPHPSL |
| 85 | NPSLNPPSYLHRAPSRIS |
| 86 | LPWRTSLLPSLPLRRRP |
| 87 | PPLFAKGPVGLLSRSFPP |
| 88 | VPPAPVVSLRSAHARPPY |
| 89 | LRPTPPRVRSYTCCPTP- |
| 90 | PNVAHVLPLLTVPWDNLR |
| 91 | CNPLLPLCARSPAVRTFP |

(S) is optional in sequences 160 to 164.

Examples of rigid linkers include the peptide sequences GAPAPAAPAPA (SEQ ID NO: 92), PPPP (SEQ ID NO: 93) and PPP.

Other linkers are shown in Table 3:

TABLE 3
Other Hinge linker sequences

| SEQ ID NO: | SEQUENCE |
|---|---|
| 94 | DLCLRDWGCLW |
| 95 | DICLPRWGCLW |
| 96 | MEDICLPRWGCLWGD |
| 97 | QRLMEDICLPRWGCLWEDDE |
| 98 | QGLIGDICLPRWGCLWGRSV |
| 99 | QGLIGDICLPRWGCLWGRSVK |
| 100 | EDICLPRWGCLWEDD |
| 101 | RLMEDICLPRWGCLWEDD |
| 102 | MEDICLPRWGCLWEDD |
| 103 | MEDICLPRWGCLWED |
| 104 | RLMEDICLARWGCLWEDD |
| 105 | EVRSFCTRWPAEKSCKPLRG |
| 106 | RAPESFVCYWETICFERSEQ |
| 107 | EMCYFPGICWM |

### Tether Format Binding Molecules and Antibodies

In one embodiment, a binding molecule, and in particular an antibody, of the invention may comprise two parts brought together by a heterodimeric tether. For example, an antibody of the invention may comprise two parts with each comprising a different antibody fragment having a different paratope for CD45 and also the tether region which allows it to form the overall antibody molecule with the other half of the antibody. In one embodiment, the antibody of the invention is in the Fab-X/Fab-Y antibody format (also referred to as the Fab-Kd-Fab format). The Fab-X/Fab-Y antibody format is particularly useful for screening because it allows permutations of different paratopes for CD45 to be rapidly screened.

Hence, in one embodiment an antibody molecule according to the present invention is an antibody comprising at least two different paratopes specific for different epitopes of CD45 having the formula A-X:Y-B wherein:
A-X is a first fusion protein;
Y-B is a second fusion protein;
X:Y is a heterodimeric-tether;
: is a binding interaction between X and Y;
A is a first protein component of the antibody selected from a Fab or Fab′ fragment;
B is a second protein component of the antibody selected from a Fab or Fab′;
X is a first binding partner of a binding pair independently selected from an antigen or an antibody or binding fragment thereof; and
Y is a second binding partner of the binding pair independently selected from an antigen or an antibody or a binding fragment thereof;
   with the proviso that when X is an antigen Y is an antibody or binding fragment thereof specific to the antigen represented by X and when Y is an antigen X is an antibody or binding fragment thereof specific to the antigen represented by Y.

### Illustrative Examples of CD45 Antibodies and Sequences

Any suitable paratopes specific for CD45 may be employed in the present invention. Illustrative examples of such antibodies are set out herein.

In one embodiment, an antibody of the present invention may comprise at least one of the following CDRs:

CDRH1
SEQ ID NO: 1
GFSFSGNYYMC
CDRH1 variant
SEQ ID NO: 2
GFSFSGNYYMS
CDRH2
SEQ ID NO: 3
CLYTGSSGSTYYASWAKG
CDRH2 variant
SEQ ID NO: 4
SLYTGSSGSTYYASWAKG
CDRH3
SEQ ID NO: 5
DLGYEIDGYGGL
CDRH3 variant 1
SEQ ID NO: 6
DLGYEIDSYGGL
CDRH3 variant 2
SEQ ID NO: 7
DLGYEIDAYGGL
CDRH3 variant 3
SEQ ID NO: 8
DLGYEIDTYGGL
CDRL1
SEQ ID NO: 9
QASQSVYNNNNLS
CDRL1 variant 1
SEQ ID NO: 10
QASQSVYNNNSLS
CDRL1 variant 2
SEQ ID NO: 11
QASQSVYNNNQLS
CDRL1 variant 3
SEQ ID NO: 12
QASQSVYNNNNLA
CDRL2
SEQ ID NO: 13
DASKLAS
CDRL3
SEQ ID NO: 14
LGGYYSSGWYFA

For instance, in one embodiment, an antibody may comprise at least one, two, three, four, five, or six CDRs from SEQ ID NOs: 1 to 14. In one embodiment, it may comprise at least one CDR1, CDR2, and/or CDR3 sequences from those set out in SEQ ID NOs: 1 to 14. In another embodiment, an antibody of the invention may comprise a heavy chain variable region comprising a CDRH1, CDRH2, and/or CDRH3 sequence from those set out in SEQ ID NOs: 1 to 8. In another embodiment, an antibody of the invention may comprise a light chain variable region comprising a CDRL1, CDRL2, and/or CDRL3 sequence from those set out in SEQ ID NOs: 9 to 14. In one embodiment, an antibody may comprise six CDRs from SEQ ID NOs: 1 to 14, where a CDRH1, CDRH2, CDRH3, CDRL1, CDRL2, and CDRL3 are selected from the corresponding CDR sequences of SEQ ID NOs: 1 to 14, where a particular CDR may be the original CDR of the 4133 specificity, or one of the variant sequences set out in SEQ ID NOs: 1 to 14. In one embodiment, an antibody may comprise a paratope comprising the CDRs of SEQ ID Nos: 1, 3, 5, 9, 13, and 14. In one embodiment, an antibody of the invention comprises at least one paratope against CD45 which comprises such CDR sequences. In one embodiment, the present invention provides the CD45 antibodies described herein in any suitable antibody format. Accordingly in one embodiment the present invention provides anti-CD45 antibodies or fragments thereof containing one or more of the binding domains described herein comprising the CDRs may be in any of the antibody formats set out herein.

In one embodiment, an antibody of the invention may comprise any of the variable regions of SEQ ID NOs 17 to 22. In one embodiment, one paratope of the antibody comprises a light chain variable region selected from SEQ ID No: 17 or 18. In another embodiment, one paratope of the antibody comprises a heavy chain variable region selected from SEQ ID NOs: 19 to 22. In one embodiment, a heavy chain variable region listed herein is employed in combination with a light chain variable region listed herein. In one preferred embodiment, the antibody of the invention comprises a paratope specific for CD45 comprising a light chain variable region selected from SEQ ID No: 17 or 18 and a heavy chain variable region selected from SEQ ID NOs: 19 to 22.

In one embodiment, the above are used in an antibody of the present invention which has at least two specificities for CD45, or a mixture of antibodies. In one embodiment, the antibody employed does not comprise one of the above sequences.

In one embodiment, an antibody of the present invention may comprise at least one of the following CDRs:

CDRH1
SEQ ID NO: 23
GYTFTSYTMH
CDRH2
SEQ ID NO: 24
YINPSSGYTEYNQKFKD
CDRH3
SEQ ID NO: 25
VGDGFYPSWLAY
CDRH3 variant 1
SEQ ID NO: 26
VGDSFYPSWLAY
CDRH3 variant 2
SEQ ID NO: 27
VGDAFYPSWLAY
CDRH3 variant 3
SEQ ID NO: 28
VGDTFYPSWLAY
CDRL1
SEQ ID NO: 29
KASQSVRNDVA
CDRL2
SEQ ID NO: 30
YASKRYT
CDRL3
SEQ ID NO: 31
QQDYSSPTT

For instance, in one embodiment, an antibody may comprise at least one, two, three, four, five, or six CDRs from SEQ ID NOs: 23 to 31. In one embodiment, it may comprise at least one CDR1, CDR2, and/or CDR3 sequences from those set out in SEQ ID NOs: 23 to 31. In another embodiment, an antibody of the invention may comprise a heavy chain variable region comprising a CDRH1, CDRH2, and/or CDRH3 sequence from those set out in SEQ ID NOs: 23 to 28. In another embodiment, an antibody of the invention may comprise a light chain variable region comprising a CDRL1, CDRL2, and/or CDRL3 sequence from those set out in SEQ ID NOs: 29 to 31. In one embodiment, an antibody may comprise six CDRs from SEQ ID NOs: 23 to 31, where a CDRH1, CDRH2, CDRH3, CDRL1, CDRL2, and CDRL3 are selected from the corresponding CDR sequences of SEQ ID NOs: 23 to 31, where a particular CDR may be the original CDR of the 6294 specificity or one of the variant sequences set out in SEQ ID NOs: 23 to 31. In one embodiment, an antibody of the invention comprising one paratope against CD45 which comprises such CDR sequences. In one embodiment, a paratope of an antibody of the invention comprises the CDR sequences of SEQ ID Nos: 23 to 25 and 29 to 31. In one embodiment, the present invention provides the CD45 antibodies described herein in any suitable antibody format. Accordingly, in one embodiment the present invention provides anti-CD45 antibodies or fragments thereof containing one or more of the binding domains described herein comprising the CDRs may be in any of the antibody formats set out herein.

In one embodiment, an antibody of the invention may comprise any of the variable regions of SEQ ID NOs 32 to 37. In one embodiment, one paratope of the antibody comprises a light chain variable region selected from SEQ ID No: 36 and 37. In another embodiment, one paratope of the antibody comprises a heavy chain variable region selected from SEQ ID NOs: 34 or 35. In one embodiment a heavy chain variable region listed herein is employed in combination with a light chain variable region listed herein. In one preferred embodiment, the antibody of the invention comprises a paratope specific for CD45 comprising a light chain variable region selected from SEQ ID No: 36 and 37 and a heavy chain variable region selected from SEQ ID NOs: 34 and 35.

In one embodiment of the invention, one paratope of the antibody of the invention comprises sequences of, or derived from, SEQ ID NOs: 1 to 22 as set out above, and the other paratope of the antibody comprises sequences of, or derived from SEQ ID NOs: 23 to 37 as set out above. For example, in one embodiment, one paratope of the antibody of the invention specific for CD45 comprises one, two, three, four, five, or six CDRs from SEQ ID Nos 1 to 14 as discussed above and the other paratope of the antibody comprises one, two, three, four, five, or six CDRs from SEQ ID Nos 23 to 31 as discussed above. In another embodiment, one paratope of the antibody of the invention comprises variable region sequences of, or derived from, SEQ ID Nos 17 to 21 as described above and the other paratope specific for CD45 comprise variable region sequences of, or derived from SEQ ID Nos: 34 to 37 as discussed above.

In one particularly preferred embodiment, at least one paratope of an antibody of the invention specific for CD45 comprises one, two, three, four, five, or six CDRs from the 4133 CD45 specificity discussed herein. In another particularly preferred embodiment, at least one paratope of the antibody comprises a light chain variable region for the 4133 specificity selected from SEQ ID Nos: 17 and 18. In another particularly preferred embodiment, at least one paratope of the antibody specific for CD45 comprises a heavy chain variable region for the 4133 specificity. Selected from SEQ ID Nos: 19 to 21. In another particularly preferred embodiment, at least one paratope of the antibody specific for CD45 comprises such light and heavy chain variable region sequences. In one embodiment, rather than one of the specific CDR or variable region sequences set out herein, the antibody of the present invention comprises a CDR or variable region with a sequence at least 95% identical or similar (such as 96, 97, 98, 99% identical or similar) to the specific sequence identified herein. For instance, it may have one or more amino acid sequence changes and in particular conservative sequence changes. In one embodiment, an antibody of the invention may comprise one of the framework modifications set out in the Examples, in particular Example 13.

In one embodiment the heavy chain variable region human framework employed in a paratope of antibody of the present invention is selected from the group comprising IGHV3-21, IGHV4-4, and a variant of any one of the same wherein one, two, three, four, five, six, seven, eight, nine, ten, eleven or more amino acids are substituted with an amino acid other than cysteine, for example substituted with a residue in the corresponding location in the donor antibody, for example from the specific donor VH sequences set out herein. In another embodiment, the framework is selected from the group comprising IGHV3-48, IGHV1-19, or a variant, of any one of the same wherein one, two, three, four, five, six, seven, eight, nine, ten, eleven or more amino acids are substituted with an amino acid other than cysteine, for example substituted with a residue in the corresponding location in the donor antibody, for example from the specific donor VH sequences set out herein. In one embodiment, the human framework further comprises a suitable J region sequence, such as the JH4 or JH1 J region.

In one embodiment a human VH framework employed in an antibody molecule of the present disclosure has an amino acid substituted in at least one position such as at 1, 2, 3, 4, 5, 6, 7, 8, 9, 10 or 11 positions selected from the group comprising 24, 37, 44, 48, 49, 67, 69, 71, 73, 76 and 78, for example wherein the original amino acid in the human framework is substituted for another amino acid other than cysteine, in particular substituted with a residue in the corresponding location in the framework of the donor antibody.

In one embodiment when the VH framework is type IGHV3 then substitutions may be made in at least five positions (usually five or six positions) selected from 48, 49, 69, 71, 73, 76 and 78, such as 48, 71, 73, 76 and 78 (in particular suitable for IGHV3-7), or 48, 69, 71, 73, 76 and 78 (in particular suitable for IGHV3-7), or 48, 49, 71, 73, 76 and 78 (in particular suitable for IGHV3-21). In one embodiment when the VH framework is a type IGHV4 then substitutions may be made in one or more (1, 2, 3, 4, 5, 6 or 7), such as at 5 positions selected from 24, 37, 48, 49, 67, 69, 71, 73, 76 and 78, for example in all of the positions 24, 71, 73, 76 and 78, and optionally in addition 48 and 67 (which is particularly suitable for IGHV4-4) or all the positions 24, 37, 49, 67, 69, 71, 73, 76 and 78 (which is particularly suitable for IGHV4-31).

In one embodiment, an antibody of the invention may comprise a light chain variable region where amino acids 2, 3, and/or 70 from the original framework the CDRs originate from are also transferred. For instance, in the case of the 4133 light chain variable region, the Glutamine (Q2), Valine (V3) and/or Glutamine (Q70) from the original antibody may also be transferred into the humanised light chain variable region employed, particularly where the recipient framework is IGKV1D-13. In some embodiments, CDRL1 may be mutated to remove a potential N-glycosylation site In one embodiment, in the case of the 4133 heavy chain variable region in one embodiment residues at the 48, 49, 71, 73, 76 and/or 78 positions may be transferred as well as one or more CDRs into a new framework. For instance, Isoleucine (148), Glycine (G49), Lysine (K71), Serine (S73), Threonine (T76) and/or Valine (V78), respectively may also be transferred, particularly where the acceptor framework is IGHV3-21. In some cases, CDRH1 and CDRH2 may be mutated to remove Cysteine residues (CDRH1 variant and CDRH2 variant, respectively). CDRH3 may also be mutated to modify a potential Aspartic acid isomerisation site (CDRH3 variant 1-3). In another embodiment, where CDRs from the 4133 heavy chain variable region are used, one or more of the following framework residues from the 4133 VH gene (donor residues) may be retained at positions 24, 71, 73, 76 and 78, particularly where the acceptor framework is a IGHV4-4 sequence. For instance, Alanine (A24), Lysine (K71), Serine (S73), Threonine (T76) and Valine (V78), respectively may also be transferred as well as CDRs. In some cases, CDRH1 and CDRH2 may be mutated to remove Cysteine residues (CDRH1 variant and CDRH2 variant, respectively). CDRH3 may also be mutated to modify a potential Aspartic acid isomerisation site (CDRH3 variant 1-3). Example 13 of the present application also sets out framework residues which may be retained where the 6294 specificity is used as the source of CDRs and retention of those residues may be, in some embodiments employed, where one or more CDR is from the 6294 specificity.

In one preferred embodiment, the human framework further comprises a suitable human J region, such as a JH1 or a JH4 J region. In one preferred embodiment, a JH1 J region is employed.

Kabat numbering is employed herein unless the context indicates otherwise.

In one embodiment the light chain variable region human framework employed in the humanised antibody molecule of the present disclosure is selected from the group comprising IGKV1-5, IGKV1-12, IGKV1D-13 and a variant of any one of the same wherein one, two, three, four or five amino acids (such as 2 amino acids) are substituted with an amino acid other than cysteine, for example substituted with a donor residue in the corresponding location in the original donor antibody, for example from the donor V_{L} sequences provided in SEQ ID NO:60, 69, 78 or 88. Typically the human framework further comprises a suitable human J region sequence, such as the JK4 J region.

In one embodiment the change or changes in the light and/or heavy chain frameworks are shown in the sequences listed herein.

In one embodiment a human VL framework employed in an antibody molecule of the present disclosure has an amino acid substituted in at least one position selected from the group comprising 2, 3, and 70, for example wherein the original amino acid in the human framework is substituted for another amino acid other than cysteine, in particular substituted for a residue in the corresponding location in the framework of the donor antibody. In one embodiment, when an IGKV1D-13 human framework is employed one, two or three substitutions may be made at positions independently selected from 2, 3 and 70. In one embodiment, after substitution position 2 of the VL framework is glutamine. In one embodiment after substitution position 3 of the VL framework is valine. In one embodiment after substitution position 70 of the VL framework is glutamine.

It will be appreciated that one or more of the substitutions described herein may be combined to generate a humanised VL region for use in an antibody molecule of the present invention.

In one independent aspect there is provided a humanised VL variable domain comprises a sequence independently selected from SEQ ID NO: 17 and 18 and a humanised sequence at least 95% identical or similar to any one of the same (such as 96, 97, 98 or 99% identical or similar to any one of the same). In an alternative embodiment the humanised VL variable domain comprises a sequence independently selected from SEQ ID NO: 36, 37, and a humanised sequence at least 95% identical or similar to any one of the same (such as 96, 97, 98 or 99% identical or similar to any one of the same).

In one independent aspect there is provided a humanised VH variable domain comprising a sequence independently selected from SEQ ID NO: 19 to 22 and a humanised sequence at least 95% identical or similar to any one of the same (such as 96, 97, 98 or 99% identical or similar) and a humanised VL variable domain comprising a sequence independently selected from SEQ ID NO: 17, 18, and a humanised sequence at least 95% identical or similar to any one of the same (such as 96, 97, 98 or 99% identical or similar to any one of the same). In one alternative aspect there is provided a humanised VH variable domain comprising a sequence independently selected from SEQ ID NO: 34, 35, and a humanised sequence at least 95% identical or similar to any one of the same (such as 96, 97, 98 or 99% identical or similar) and a humanised VL variable domain comprising a sequence independently selected from SEQ ID NO: 36, 37, and a humanised sequence at least 95% identical or similar to any one of the same (such as 96, 97, 98 or 99% identical or similar to any one of the same).

In one independent aspect there is provided a humanised VH variable domain comprising a sequence independently selected from SEQ ID NO: 19 to 22 and a humanised VL variable domain comprising a sequence independently selected from SEQ ID NO: 17 and 18. In one alternative aspect there is provided a humanised VH variable domain comprising a sequence independently selected from SEQ ID NO: 34 and 35 and a humanised VL variable sequence independently selected from SEQ ID NO: 36 and 37, or a variant where the heavy chain CDR3 is selected from SEQ ID NO: 26, 27, and 28. For instance, in one preferred embodiment, the antibody provided comprises the HCDR1 sequence of SEQ ID NO: 23, the HCDR2 sequence of SEQ ID NO: 24, and a variant HCDR3 sequence selected from one of SEQ ID Nos: 26, 27, and 28. In a preferred embodiment, the antibody provided comprises a LCDR1 sequence of SEQ ID NO: 29, a LCDR2 sequence of SEQ ID NO: 30, and a LCDR3 sequence of SEQ ID NO: 31. In another embodiment, the antibody comprises both such heavy chain and light chain CDR sequences, so a heavy chain variable region comprising the HCDR1 of SEQ ID NO: 23, the HCDR2 sequence of SEQ ID NO: 24, and a variant HCDR3 sequence selected from one of SEQ ID Nos: 26, 27, and 28, as well as a light chain comprising the LCDR1 of SEQ ID NO: 29, the LCDR2 of SEQ ID NO 30, and the LCDR3 of SEQ ID NO: 31. The present invention also comprises an antibody comprising a set of such six CDRs in general that is not limited to a biparatopic antibody.

Also provided is an antibody or binding fragment comprising a paratope that binds the same epitope as the paratope of an antibody or binding fragment explicitly disclosed herein.

In one preferred embodiment, an antibody of the present invention comprises a light chain having the sequence of SEQ ID NO: 115 or a sequence which is at least 95% identical or similar to the same (such as 96, 97, 98 or 99% identical or similar). In another embodiment, an antibody of the present invention comprises a light chain having the sequence of SEQ ID NO: 118 or a sequence with is at least 95% identical or similar to the same (such as 96, 97, 98 or 99% identical or similar). In another preferred embodiment, an antibody of the present invention, in particular an antibody with two different specificities, will comprise both such light chains.

In another preferred embodiment, an antibody of the present invention will comprise a heavy chain having the sequence of SEQ ID NO: 116 or a sequence which is at least 95% identical or similar to the same (such as 96, 97, 98 or 99% identical or similar to any one of the same), preferably whilst retaining the S228P, F234A, L235A modifications. In another embodiment, an antibody of the present invention will comprise a heavy chain having the sequence of SEQ ID NO: 117 or a sequence with is at least 95% identical or similar to any one of the same (such as 96, 97, 98 or 99% identical or similar to any one of the same), preferably whilst retaining the S228P, F234A, L235A and T355W modifications).

In another preferred embodiment, an antibody of the present invention will comprise a heavy chain having the sequence of SEQ ID NO: 119 or a sequence which is at least 95% identical or similar to the same (such as 96, 97, 98 or 99% identical or similar), preferably whilst retaining the S228P, F234A, L235A, T366S, L368A, and Y407V modifications.

In one embodiment, an antibody of the present invention will comprise two of the heavy chains and light chains set out above of SEQ ID NOs: 115 to 119 or sequences with at least 95% identity or similarity to the same (such as at least 96, 97, 98 or 99% identity or similarity) whilst retaining the stated modifications for FALA and/or knobs-in-holes. In one embodiment, an antibody of the present invention will have the light chains of SEQ ID NOs: 115 and 118 and the heavy chains of SEQ ID No: 117 and 119. In another embodiment, it will have at least 95% identity or similar to any one, or all of, the same (such as being 96, 97, 98 or 99% identical or similar to any one of the same) whilst retaining the FALA and knob-in-hole modifications.

The present invention also provides antibodies that comprise the CDRs, variable regions, light chain and/or heavy chain sequences of the 6294 antibody, or a variant of the 6294 sequences, without the antibody having to be necessarily biparatopic for CD45. For instance, the present invention provides an antibody where one of the specificities of the antibody comprises the sequences of the 6294 antibody or a variant thereof. In another embodiment, a monospecific antibody comprising such sequences of the 6294 antibody is provided. For example, such antibodies, may comprise six CDRs from SEQ ID NOs: 23 to 31, where a CDRH1, CDRH2, CDRH3, CDRL1, CDRL2, and CDRL3 are selected from the corresponding CDR sequences of SEQ ID NOs: 23 to 31, where a particular CDR may be the original CDR of the 6294 specificity or one of the variant sequences set out in SEQ ID NOs: 23 to 31. In one alternative aspect there is provided an antibody comprising a humanised VH variable domain comprising a sequence independently selected from SEQ ID NO: 34 and 35 and a humanised VL variable sequence independently selected from SEQ ID NO: 36 and 37. The present invention also provides the 6294 antibody as well as humanised versions and other variants of it. The various related aspects to antibodies set out herein, such as vectors, nucleic acids, pharmaceutical compositions and so on may also be employed for such antibodies.

### Illustrative Examples of Albumin Antibodies and Sequences

An antibody specific for albumin used in the present invention may have the following CDR sequences:

CDRH1
SEQ ID NO: 120
GIDLSNYAIN
CDRH2
SEQ ID NO: 121
IIWASGTTFYATWAKG
CDRH3
SEQ ID NO: 122
TVPGYSTAPYFDL
CDRL1
SEQ ID NO: 123
QSSPSVWSNFLS
CDRL2
SEQ ID NO: 124
EASKLTS
CDRL3
SEQ ID NO: 125
GGGYSSISDTT

Such an antibody may comprise a VL sequence of SEQ ID NO: 126 and a VH sequence of SEQ ID NO: 127. Such an antibody may alternatively comprise disulphide-linked VL and VH sequences of SEQ ID NO: 128 and SEQ ID NO: 129 respectively. In the case of a TrYbe comprising two CD45 paratopes and an albumin paratope, the heavy chain may comprise the sequence of SEQ ID NO: 130 and the light chain may comprise the sequence of SEQ ID NO: 131.

### Effector Molecules

A binding molecule, an in particular an antibody, of the invention may be conjugated to an effector molecule. Hence, if desired a binding molecule, and in particular an antibody, for use in the present invention may be conjugated to one or more effector molecule(s). It will be appreciated that the effector molecule may comprise a single effector molecule or two or more such molecules so linked as to form a single moiety that can be attached to the binding molecules, an in particular antibodies, of the present invention. Where it is desired to obtain a binding molecule, and in particular an antibody, according to the present invention linked to an effector molecule, this may be prepared by standard chemical or recombinant DNA procedures in which the binding molecule, and in particular antibody, is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745. In one embodiment the binding molecules, in particular antibodies, of the present invention may comprise an effector molecule. The term effector molecule as used herein includes, for example, antineoplastic agents, drugs, toxins, biologically active proteins, for example enzymes, antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin. Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, Pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another embodiment, the effector molecule may increase or decrease the half-life of the binding molecule, in particular antibody, in vivo, and/or reduce immunogenicity and/or enhance delivery across an epithelial barrier to the immune system. Examples of suitable effector molecules of this type include polymers, albumin, albumin-binding proteins or albumin binding compounds such as those described in WO 05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide. Specific optional substituents which may be present on the above-mentioned synthetic polymers include one or more hydroxy, methyl or methoxy groups. Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

A binding molecule, in particular an antibody, of the present invention may be conjugated to a molecule that modulates or alters serum half-life. A binding molecule, in particular an antibody, of the invention may bind to albumin, for example in order to modulate the serum half-life. In one embodiment, a binding molecule, in particular an antibody, of the invention will also include a paratope specific for albumin. In another embodiment, a binding molecule, in particular an antibody, of the invention may include a peptide linker which is an albumin binding peptide. Examples of albumin binding peptides are included in WO2015/197772 and WO2007/106120 the entirety of which are incorporated by reference.

In another embodiment, a binding molecule, in particular an antibody, of the invention is not conjugated to an effector molecule. In one embodiment, a binding molecule, in particular an antibody, of the invention is not conjugated to a toxin. In another embodiment, a binding molecule, in particular an antibody, of the invention is not conjugated to a radioisotope. In another embodiment, a binding molecule, in particular an antibody, of the invention is not conjugated to an agent for imaging.

In one preferred embodiment, it is the ability of a binding molecule, in particular an antibody, of the present invention to bind CD45 that brings about cell death (preferably apoptosis) and not the ability of a conjugated effector molecule. In one preferred embodiment, it is the ability to cross-link CD45 that brings about cell death (preferably apoptosis).

### Cell Death and Killing

In one especially preferred embodiment, a binding molecule of the present invention, and in particular an antibody of the present invention, is able to induce cell death in a target cell. Types of cell death that may be induced to kill target cells include intrinsic apoptosis, extrinsic apoptosis, mitochondrial permeability transition (MPT)-driven necrosis, necroptosis, ferroptosis, pyroptosis, parthanatos, entotic cell death, NETotic cell death, lysosome-dependent cell death, autophagy-dependent cell death, immunogenic cell death, cellular senescence, and mitotic catastrophe. In an especially preferred embodiment, a binding molecule of the present invention, and in particular an antibody of the present invention, is used to induce apoptosis. In one embodiment, a binding molecule of the present invention, and in particular an antibody, is used to kill target cells.

In one embodiment, the target cell will be a cell expressing CD45, and in particular on the surface of the cell. In one preferred embodiment, a binding molecule of the present invention, and in particular an antibody of the present invention, may induce cell death (preferably apoptosis) in at least T cells. In another preferred embodiment, a binding molecule of the present invention, and in particular an antibody of the present invention, may induce cell death (preferably apoptosis) in at least B cells. In another preferred embodiment, a binding molecule of the present invention, and in particular an antibody of the invention, may be able to induce cell death (preferably apoptosis) in B and T cells. In one preferred embodiment, a binding molecule of the present invention, and in particular an antibody of the present invention, is able to induce cell death (preferably apoptosis) in haematopoietic stem cells. In one embodiment, a binding molecule of the present invention, and in particular an antibody of the present invention, does not induce cell death in all immune cells, for example, not granulocytes, macrophages and monocytes. In one embodiment, a binding molecule of the present invention, and in particular an antibody of the invention, induces cell death in all immune cells apart from granulocytes, macrophages and monocytes. In one embodiment, the effect of inducing cell death in haematopoietic stem cells is effectively that all haematopoietic cells can be replaced. In one embodiment, a binding molecule of the present invention is used to kill the above mentioned target cells by inducing cell death.

In another particularly preferred embodiment a binding molecule of the present invention, and in particular an antibody of the present invention, induces cell death (preferably apoptosis), but does not bring about significant cytokine release. In another preferred embodiment, a binding molecule of the present invention, and in particular an antibody of the invention, induces cell death (preferably apoptosis), but does not display Fc effector functions, for example because the antibody lacks an Fc region or has an Fc region with silencing modifications.

### Cytokines

In one particularly preferred embodiment, a binding molecule, and in particular an antibody, of the present invention does not bring about significant cytokine release. In an especially preferred embodiment, a binding molecule, and in particular an antibody, of the present invention is able to induce cell death in a target cell, but does not bring about significant cytokine release. The reduction or absence of cytokine release may mean that a subject does not suffer unwanted cytokine driven inflammation. For instance, a treatment of the present invention may kill target cells in a subject without trigging inflammation and in particular without a so-called “cytokine storm” associated with some treatments.

In one embodiment, a binding molecule of the present invention, and in particular an antibody of the present invention, does not significantly induce the release of one or more of Interferon-gamma, IL-6, TNF-alpha, IL-1Beta, MCP1 and IL-8. In one preferred embodiment, a binding molecule, in particular an antibody, of the invention does not bring about significant release of any of those cytokines. In another embodiment, a binding molecule of the invention, in particular an antibody, does not significantly induce the release of one or more of CCL2, IL-1RA, IL-6, and IL-8. In another preferred embodiment, it does not significantly induce release of any of those cytokines. In one embodiment, such levels will be the case for one or more of Interferon-gamma, IL-6, TNF-alpha, IL-1Beta, MCP1 and IL-8. In another embodiment, such levels will be the case for one or more of CCL2, IL-1RA, IL-6, and IL-8. In another embodiment such levels will be seen for at least one of CCL2, IL-1RA, IL-6, IL-8, IL-10, and IL-11. In another embodiment, such levels will be the case for at least one of CCL2, IL-1RA, IL-6, IL-8.

Cytokine release may be measured using any suitable assay. For example, the ability of a binding molecule, and in particular an antibody of the invention, to bring about cytokine release may be determined by culturing cells in vitro with the binding molecule and measuring cytokine release. In one embodiment, whole blood is incubated with the antibody and then cytokine levels measured, for example any of those cytokines mentioned above. In another embodiment, white blood cells isolated from a whole blood sample may be incubated with the binding molecule of the present invention and the level of cytokine(s) measured. Alternatively, it may be that the level of cytokine(s) is measured in a sample from a subject administered a binding molecule of the present invention, in particular cytokine level(s) may be measured in a serum sample from a subject.

In one embodiment, not “significantly inducing” cytokine release means that a binding molecule of the invention does not induce cytokine release more than five, four, three, or two fold of that seen with a negative control, for example compared to a negative control of in vitro treatment with PBS alone. In some embodiments, the level of cytokine release will be compared to a positive control, for example in vitro treatment with campath. In one embodiment, a binding molecule of the present invention will trigger not more than 50%, 40%, 30%, 20%, 10% or less compared to that seen with treatment with campath. In one embodiment, the level of cytokine release seen with a binding molecule of the present invention will be under one tenth of that seen with campath. In one embodiment, the level of cytokine release following incubation with campath will be at least double, triple, four times, five times, ten times or more that seen following incubation with a binding molecule of the present invention. In one embodiment, following incubation of whole blood for 24 hours the level seen with campath will be those levels compared to a binding molecule of the present invention. In another embodiment the comparator for defining not significantly induced will be another binding molecule. For example, where a binding molecule of the present invention comprises a modification designed to reduce cytokine release, the comparator will be the equivalent binding molecule, but without such a modification. In another embodiment, where the binding molecule is an antibody an it either has an Fc region modification intended to reduce cytokine release or no Fc region, the comparison performed is with the equivalent antibody that lacks such a modification or which has an Fc region.

In another embodiment, the comparison for not significantly releasing cytokines will be performed in vivo. For example, when a binding molecule of the present invention is given to a subject it will show any of the levels of cytokine release discussed above compared to the comparators discussed above. In another embodiment, not significantly inducing cytokine release may be in terms of the level of cytokine or cytokines compared to before administration of an antibody of the present invention. It may be, for example, that the level of a cytokine rises no more than ten-fold, fivefold, or less following administration of a binding molecule of the present invention. The measurement may be performed, for instance, immediately before or at the same time as administration of the antibody and, for example, one day, one week, or two weeks or more after administration. In one embodiment, the measurement is performed one day to one week after the administration. In another embodiment, a binding molecule of the present invention does not significantly induce cytokine release in the sense that the subject treated does not experience adverse effects associated with unwanted cytokine release, for example the subject does not experience fever, low pressure, or irregular or rapid heartbeat.

### Functional Assays

In one embodiment, a functional assay may be employed to determine if a binding molecule or binding molecules of the present invention have a particular property, for instance such as any of those mentioned herein. Hence, functional assays may be used in evaluating a binding molecule, and in particular an antibody, of the present invention. A “functional assay,” as used herein, is an assay that can be used to determine one or more desired properties or activities of the binding molecule or molecules of the present invention. Suitable functional assays may be binding assays, cell death (preferably apoptosis) assays, antibody-dependent cellular cytotoxicity (ADCC) assays, complement-dependent cytotoxicity (CDC) assays, inhibition of cell growth or proliferation (cytostatic effect) assays, cell-killing (cytotoxic effect) assays, cell-signalling assays, cytokine production assays, antibody production and isotype switching, and cellular differentiation assays. In one embodiment, an assay may measure the degree of cell depletion, for example for a specific cell type, via employing an antibody of the present invention. In one preferred embodiment, an assay may measure the ability of an antibody of the invention to induce cell death (preferably apoptosis) in target cells expressing CD45. In a further preferred embodiment, a functional assay may measure the ability of a binding molecule, in particular antibody, of the present invention to induce cytokine release. In one preferred embodiment, a functional assay may be used to determine if a binding molecule, in particular antibody, of the present invention may be used to kill cells but not significantly induce cytokines,

The functional assays may be repeated a number of times as necessary to enhance the reliability of the results. Various statistical tests known to the skilled person can be employed to identify statistically significant results and thus identify binding molecules, in particular antibodies, with biological functions. In one embodiment, multiple binding molecules are tested in parallel or essentially simultaneously. Simultaneously as employed herein refers to the where the samples/molecules/complexes are analysed in the same analysis, for example in the same “run”. In one embodiment simultaneously refers to concomitant analysis where the signal output is analysed by the instrument at essentially the same time. This signal may require deconvolution to interpret the results obtained. Advantageously, testing multiple biparatopic protein complexes allows for more efficient screening of a large number of antibodies and the identification of new and interesting relationships. Clearly, different variable regions to the target antigens of interesting CD45 can give access to subtle nuances in biological function.

In one embodiment, where a binding molecule of the present invention comprises more than one specificity for CD45, a functional assay may be used to compare the properties of that binding molecule with, for example, binding molecules having the same valency but just one of the specificities of a binding molecule of the present invention. In one embodiment, such assays may be used to show that a binding molecule of the present invention with at least two different specificities for CD45 is superior to the comparator binding molecules. Hence, in one preferred embodiment, the efficacy of binding molecules of the present invention comprising at least two different specificities for CD45, in particular such antibodies according to the present invention, can be compared to individual “comparator” binding molecules, in particular “comparator” antibodies, comprising just one of the specificities against CD45 from a binding molecule of the present invention. For example, where the assay is performed to study cross-linking, or the effect of cross-linking, of CD45, a binding molecule, in particular an antibody, having the same valency, but just one specificity may be employed as a comparator. In one embodiment, where the binding molecule is an antibody of the present invention, it may be compared with an antibody comprising the same one of the paratopes from the antibody of the invention at all of the antigen-binding sites of the antibody. In one embodiment, an antibody of the invention may be compared with one of the same valency and format as the antibody of the invention, but where the same one of the paratopes from the antibody of the invention is present at all of the antigen-binding sites. In one embodiment, a bivalent antibody comprising the two different paratopes specific for different epitopes of CD45 may be compared with each of the two possible bivalent antibodies comprising just one of those paratopes. In one embodiment, such comparisons are performed with one comparator antibody for each different specificity, in particular for each different paratope, of the antibody of the invention specific for CD45. In one embodiment, an antibody of the invention will show better results than against one such comparator antibody. In another embodiment, it will show better results than all of the comparator antibodies for each specificity, in particular paratope, of the antibody specific for CD45.

In another embodiment where the binding molecules of the present invention comprise at least two different specificities for CD45, monospecific binding molecules, in particular monospecific antibodies, are first assessed and the chosen candidates then used in the generation of an antibody of the invention with at least two different specificities against CD45. In one embodiment, multiple binding molecules, in particular antibodies, are tested by using a multiplex as defined above and subjecting the same to one or more functional assays.

Mixtures of at least two binding molecules of the present invention, in particular antibodies of the present invention, may be compared against the individual binding molecules making up a mixture of the present invention using a functional assay. In a preferred embodiment, the mixture gives superior results to any of the individual binding molecules, in particular the individual antibodies.

The term “biological function” as used herein refers to an activity that is natural to or the purpose of the biological entity being tested, for example a natural activity of a cell, protein or similar. Ideally, the presence of the function can be tested using an in vitro functional assay, including assays utilizing living mammalian cells. Natural function as employed herein includes aberrant function, such as functions associated with cancers.

In one embodiment, a binding molecule, in particular antibody, of the invention will be able to cross-link CD45 to a greater extent than a comparator binding molecule, in particular than a comparator antibody, such as those discussed above. For instance the ability of a binding molecule, in particular antibody, of the invention to form CD45 multimers of antibody:CD45 ECD may be studied when the two are mixed, such as in equal amounts. A multimer may be a structure with at least two binding molecule:CD45 ECD units. One suitable technique is mass photomotery, with the binding molecule, in particular antibody, mixed with an equal concentration of CD45 ECD, such as that of SEQ ID No: 113 and mass photometry performed on the test sample. Controls with the antibody and CD45 ECD alone may be performed. A binding molecule, and in particular an antibody, of the present invention may give rise to more multimers than a comparator binding molecule, in particular than a comparator antibody. A binding molecule, in particular an antibody, of the present invention may give rise to a greater amount of multimers with two, three, four, or more binding molecule:CD45 ECD units than the comparator. It may do so for all of the possible comparators for each of the specificities (in particular paratopes) specific for CD45. A further suitable technique for such comparison is analytical ultracentrifugation (AUC). Again, the comparison performed may also be between a mixture of binding molecules compared with each individual type of binding molecules in the mixture on their own.

In another embodiment, the comparison may be in terms of the ability of a binding molecule, in particular an antibody, of the present invention to induce cell death. In particular, in a preferred embodiment the ability of the binding molecule or molecules, in particular antibody or antibodies, to induce apoptosis may be studied. For example, a binding molecule, in particular an antibody, of the invention may induce more target cells expressing CD45 to undergo apoptosis than a comparator, for instance than a comparator antibody. It may induce a higher amount of apoptosis when measured using T cells. For example, T cells isolated from PBMC may be used. Any binding molecule, in particular antibody, of the invention may induce a higher level of apoptosis in CD4+ T cells. It may do so in CD8+ T cells. It may do so in CD4+ memory T cells. It may do so in CD4+ naïve T cells. In another embodiment, the total cell count in whole blood may be measured after incubation with a binding molecule, in particular an antibody, of the invention and compared to the results seen for a comparator. In one embodiment, a total cell count may be measured and compared for a binding molecule, in particular an antibody, of the invention with a control binding molecule, in particular antibody. In an especially preferred embodiment of the invention, Annexin V may be used to measure apoptosis. Hence, for instance, a binding molecule, in particular an antibody, of the invention will bring about a greater proportion of AnnexinV staining cells compared to the comparator.

In one embodiment in vivo assays, such as animal models, including mouse tumor models, models of auto-immune disease, virus-infected or bacteria-infected rodent or primate models, and the like, may be employed to test binding molecules of the present invention. In another embodiment, the degree of depletion of a particular cell type may be measured, for example in vivo. In one embodiment, a binding molecule, in particular an antibody, of the invention will bring about a greater level of depletion than a comparator in an animal model of a disorder and in a preferred embodiment in an animal model of cancer.

In one embodiment a binding molecule, in particular an antibody molecule, according to the present invention has a novel or synergistic function. The term “synergistic function” as used herein refers to biological activity that is not observed or higher than observed when comparator(s) are employed instead. Therefore, “synergistic” includes novel biological function. In one embodiment, a binding molecule, in particular an antibody, of the present invention comprising at least two specificities for CD45 is synergistic in that it is more effective than a binding molecule, in particular an antibody, comprising either of the specificities against CD45 individually, such as the comparators discussed above. In one preferred embodiment, such synergy is shown in relation to cross-linking of CD45. In one embodiment, a mixture of binding molecules displays synergy compared to any of the individual binding molecules making up the mixture on their own.

In one embodiment, novel biological function as employed herein refers to function which is not apparent or absent until the two or more synergistic entities [protein A and protein B] are brought together or a previously unidentified function. Higher as employed herein refers to an increase in activity including an increase from zero i.e. some activity in the binding molecule or molecules where comparator has/have no activity in the relevant functional assay, also referred to herein as new activity or novel biological function. Higher as employed herein also includes a greater than additive function in the antibody in a relevant functional assay in comparison to the individual paratopes, for example 10, 20, 30, 40, 50, 60, 70, 80, 90, 100, 200, 300% or more increase in a relevant activity.

In one embodiment the novel synergistic function is a higher inhibitory activity.

In one particularly preferred embodiment of the invention, the synergy is in relation to cell depletion of a target cell type expressing CD45. In one embodiment, synergy is in relation to cell killing.

Suitable binding domains for use in the present invention can also be identified by testing one or more binding domain pairs in a functional assay. For example, binding molecules, for example an antibody, comprising at least a binding site specific to the antigen CD45 may be tested in one or more functional assays.

In one embodiment, the ability of a binding molecule to kill CD45 expressing cancer cell lines may be assayed. Examples of cancer cell lines that may be employed in such assays for cell killing include leukaemia and lymphoma cell lines. In one embodiment, any of the following cell lines representing various leukaemia and lymphoma cell lines, as classified by ATCC (www.atcc.org/), may be used to study the ability to bring about cancer cell killing: Jurkat—acute T-cell leukaemia; CCRF-SB—B-cell acute lymphoblastic leukaemia; MC116—B-cell undifferentiated lymphoma; Raji, Ramos—Burkitt lymphoma (rare form of B-cell non-Hodgkin lymphoma); SU-DHL-4, SU-DHL-5, SU-DHL-8, NU-DUL-1, OCI-Ly3—Diffuse large B-cell lymphoma; THP-1—acute monocytic leukaemia; and Dakiki—B cell nasopharyngeal carcinoma. The methods for assessing the ability of a binding molecule to bring about killing of such cell lines employed in the Examples of the present application may be used to study the ability of a given binding molecule to kill cells. In one embodiment, a variant binding molecule of the present invention will have the same or greater ability to kill cancer cells in such assays as one of the specific binding molecules set out herein. In one embodiment, they will have at least 50%, 75%, 80%, 90%, 100% or more of the activity of one the specific binding molecules set out herein to kill one of the cancer cell lines mentioned above in such an assay. In one embodiment, a binding molecule of the present invention will kill at least 25%, 40%, 50%, 60% or 75% of cancer cells in such an assay. In another embodiment, a binding molecule of the present invention will kill 100% of the cancer cells in such an assay.

### Pathological Conditions, Medical Uses, and Cell Depletion

The present invention provides a binding molecule, in particular an antibody, of the invention for use in a method of treatment of the human or animal body. A binding molecule, in particular an antibody, of the present invention may be employed in any context where targeting CD45 may be of therapeutic benefit, in particular where killing such cells may be of therapeutic benefit. A binding molecule, in particular an antibody, of the present invention may also be used in diagnosis or detection of CD45. The present invention further provides a pharmaceutical composition of the invention for such use. The present invention also provides nucleic acid molecule(s) and vector(s) of the present invention for such uses.

Hence, the binding molecules of the present invention may be employed therapeutically. In one embodiment, rather than a binding molecule or molecules of the present invention being administered nucleic acid molecule(s) or vector(s) of the present invention may be administered to bring about expression of the binding molecule or molecules inside the target cell. In another embodiment, a pharmaceutical composition of the present invention is the preferred therapeutic administered. Whilst binding molecules, in particular antibodies, are set out as the preferred therapeutic below, pharmaceutical composition, nucleic acid molecule(s), and vector(s) of the present invention may also be employed in any of the embodiments set out. In a preferred embodiment though binding molecule(s) or a pharmaceutical composition comprising them is the preferred therapeutic. In an especially preferred embodiment, an antibody, antibodies or a pharmaceutical composition comprising them is the therapeutic.

In one particularly preferred embodiment, the present invention may be employed to deplete target cells expressing CD45. In a particularly preferred embodiment, the present invention is used to deplete a disease causing cell type expressing CD45. In particular, the present invention may be used to deplete target cells expressing CD45 on the surface of the cells. In a particularly preferred embodiment the binding molecule employed or encoded by the nucleic acid molecule or vector is one that has at least two different specificities for CD45. Whilst not being bound by any particular theory, it is thought by having at least two different specificities against CD45, in particular at least two different paratopes against different epitopes of CD45, that a binding molecule, and in particular an antibody, of the present invention is able to better bring about cross-linking of CD45 on the surface of the target cell, which in turn may bring about cell death (preferably apoptosis) more effectively. As discussed above though, such an effect may also be brought about by using mixtures of binding molecules.

In one preferred embodiment where an antibody or antibodies of the present invention are employed, the induction of cell death (preferably apoptosis) in the target cell via the antibody or antibodies of the present invention may mean that it is unnecessary for an antibody, or antibodies, of the invention to display one or more Fc region effector functions that an antibody would normally display. In one particularly preferred embodiment, an antibody, or antibodies, of the invention are therefore able to induce cell death (preferably apoptosis) in a target cell, but do not have an active Fc region. In a particularly preferred embodiment, the antibody or antibodies induce cell death, but do not induce significant cytokine release.

In a particularly preferred embodiment depletion of cells by the present invention is followed by the transfer of cells or a tissue to the subject. In a further particularly preferred embodiment, the transferred cells or tissues replace those that have been depleted using the invention. Treatment as discussed herein therefore includes, rather than targeting the actual mechanism of the disorder, replacing wholly or partially, a cell type involved in the disorder or one whose killing, in particular replacement, can simply have therapeutic benefit. In one embodiment, the invention therefore provides a method of depleting cells comprising employing the invention. In another embodiment, a method of the invention may comprise both depleting cells and the subsequent transfer of cells or tissue. Cell depletion may be used in a number of therapeutic contexts, effectively to kill target cells.

In a preferred instance, a binding molecule, in particular an antibody, of the invention is used to kill immune cells. As used herein, the term “immune cell” is intended to include, but is not limited to, a cell that is of hematopoietic origin and that plays a role in the immune response. In one embodiment the invention is employed to deplete T cells in a subject. In one embodiment the invention is employed to deplete B cells in a subject. In another embodiment, the invention is employed to deplete both. In another embodiment, the invention is used to deplete T cells, but not macrophages. In another embodiment, the invention is used to deplete B cells, but not macrophages. In another embodiment, the invention is used to deplete B and T cells, but does not result in the depletion of macrophages. In one embodiment, the present invention is used to deplete haematopoietic stem cells (HSCs). In another embodiment, the invention is employed to deplete haemopoietic stem cells. In one preferred embodiment, HSCs are depleted via the invention in a subject prior to the transfer of HSC to repopulate the immune system of the subject. In another embodiment, the invention depletes a particular cell type, but does not deplete haemopoietic stem cells. In another embodiment, the invention is used to kill the above-mentioned cell types. Hence, in any of the embodiments mentioned herein for cell depletion, the invention may be employed to kill the stated cells.

In one embodiment, the subject treated via the invention is one with an autoimmune disease, a blood disease, a metabolic disorder, cancer, or an immunodeficiency (such as a severe combined immune deficiency or SCID). The ability to treat conditions through first depleting and then replacing cells means that the a binding molecules, in particular antibodies, of the invention are particularly useful in treating cancers. In a particularly preferred embodiment, the disorder to be treated is therefore a cancer. In one embodiment, the invention is therefore employed to deplete cancer cells, for instance cancer cells originating from immune system cells. In one preferred embodiment, the invention provides a method of treating a cancer comprising administering the invention is employed to deplete cancer cells expressing CD45. The method may further comprise transplantation of cells to the subject. In one embodiment, the transferred cells replace the depleted cells. In one embodiment, the transferred cells are haematopoietic stem cells.

In one particularly preferred embodiment the disorder to be treated is a blood cancer. In one preferred embodiment, the cancer is one involving the bone marrow and in particular one involving cells of the haematopoietic system.

In one preferred embodiment, the cancer may be a leukaemia. In one embodiment, the leukaemia may be a childhood leukaemia. In another embodiment, the leukaemia may be an adult leukaemia. The leukemia may be an acute leukaemia. The leukaemia may be a chronic leukaemia. Non-limiting examples of leukaemias that may be treated via employing the invention include lymphocytic leukaemia (such as acute lymphoblastic leukemia or chronic lymphocytic leukemia) and myelogenous leukaemia (such as acute myelogenous leukaemia or chronic myelogenous leukaemia). The leukaemia may be a B-cell leukaemia such as, for example, B-cell acute lymphocytic leukaemia, B-cell acute lymphoblastic leukaemia, or B-cell prolymphocytic leukaemia. In one embodiment, the invention may be used to treat adult acute lymphoblastic leukaemia, childhood acute lymphoblastic leukaemia, refractory childhood acute lymphoblastic leukaemia, acute lymphocytic leukaemia, prolymphocytic leukaemia, chronic lymphocytic leukaemia, or acute myeloid leukaemia.

In one embodiment of the invention the blood cancer to be treated may be a lymphoma. Non-limiting examples of lymphoma that can be treated include B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, and refractory non-Hodgkin lymphoma.

In one embodiment, the blood cell cancer to be treated is a myeloma. Non-limiting examples of myelomas that may be treated include recurrent plasma cell myeloma, refractory plasma cell myeloma, multiple myeloma, relapsed or refractory multiple myeloma, and multiple myeloma of bone.

In one embodiment, the cancer may be one selected from an acute T-cell leukaemia, a B cell acute lymphoblastic leukaemia, an acute monocytic leukaemia and a B cell nasopharyngeal carcinoma. In another embodiment, the cancer may be one selected from an acute T-cell leukaemia, a B cell acute lymphoblastic leukaemia, a diffuse large B-cell lymphoma, an acute monocytic leukaemia and a B cell nasopharyngeal carcinoma. In another embodiment, the cancer may be one selected from an acute T-cell leukaemia, a B cell acute lymphoblastic leukaemia, a diffuse large B-cell lymphoma, an acute monocytic leukaemia, a B cell nasopharyngeal carcinoma, a B cell undifferentiated lymphoma, and a Burkitt lymphoma. In another embodiment, the cancer may be one selected from an acute T-cell leukaemia, a B cell acute lymphoblastic leukaemia, an acute monocytic leukaemia, a B cell nasopharyngeal carcinoma, a B cell undifferentiated lymphoma, and a Burkitt lymphoma. In one preferred embodiment, where such cancers are treated the binding molecule of the invention employed is a BYbe antibody.

In another embodiment, the subject to be treated has an autoimmune disorder. In one particularly preferred embodiment, the autoimmune disorder is multiple sclerosis. In another particularly preferred embodiment, the condition is scleroderma. Further examples of autoimmune diseases include scleroderma, ulcerative colitis, Crohn's disease, Type 1 diabetes, or another autoimmune pathology described herein. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

In one embodiment, the condition to be treated is one known to involve abnormal CD45 expression. In one particularly preferred embodiment, the treatment depletes a cell type expressing CD45 that plays a role in a disorder in the subject. Examples of such diseases include Alzheimer's disease, multiple sclerosis, and lupus. Other conditions known to involve alterations in CD45 expression include immunodeficiency's, such as severe combined immunodeficiency (SCID).

In one embodiment, the invention is used to deplete cells in advance of a cell transplant, hence a method of the invention may include, in some embodiments, a depletion step employing a therapeutic, in particular binding molecule and especially an antibody, of the present invention followed by a step of transferring cells to the subject, for instance to help replace the depleted cells. In one embodiment, such a transfer may be of allogenic cells. In another embodiment, such a transfer may be of autologous cells. In one embodiment, the transferred cells may be cells expressing a chimeric antigen receptor (CAR). In some embodiments, the subject is in need of chimeric antigen receptor T-cell (CART) therapy. For instance, such therapy may form part of a method of the present invention.

In another preferred embodiment, the invention provides a method of promoting the engraftment of a cell population in a subject, where the method further comprises employing a binding molecule, in particular an antibody, of the invention to deplete cells prior to the engraftment of a cell population. The present invention therefore provides a method of promoting engraftment of transferred cells comprising depleting cells expressing CD45 in a subject via administering a binding molecule, in particular an antibody, of the invention and then transferring the cells of interest. In one embodiment, the present invention provides a method for promoting the engraftment of stem cells and in particular hematopoietic stem cells. In one embodiment, hematopoietic stem cells are administered to a subject defective or deficient in one or more cell types of the hematopoietic lineage in order to re-constitute, or partially re-constitute, the defective or deficient population of cells in vivo. In one embodiment, the invention is used to treat a stem cell deficiency, for instance where the invention is used to deplete target cells and replace them with transplanted cells, where the transplanted cells address the stem cell deficiency. In one embodiment, the reintroduced cells have been genetically modified. In one embodiment, cells from the subject have been removed and genetically modified then returned to the subject after the invention has been used to kill target cells, for instance the unmodified cells of that type still present in the subject. In one preferred embodiment, the transferred cells that have been genetically modified are haematopoietic stem cells.

In one preferred embodiment, the depleted cells and the transferred cells are, or comprise, the same cell type. In one preferred embodiment, the depleted cells are haematopoietic cells and in particular haematopoietic stem cells. In one embodiment, the present invention is employed to deplete cells prior to a bone marrow transplant. In another embodiment, the present invention is employed instead of irradiation to deplete cells. In another embodiment, it is employed in addition to irradiation to deplete cells.

In another embodiment, the present invention provides a method of helping reducing the chances of rejection of transplanted cells, the method comprising administering a therapeutic of the invention to deplete cells prior to the transfer of the cells. In another embodiment, the invention may be employed to promote the acceptance of transplanted immune cells in a subject by depleting target cells expressing CD45 prior to the transfer of the immune cells. Target cells may be any of those discussed herein. In one embodiment, the cells transplanted or transferred to a subject are stem cells.

Any of the ways discussed herein to eliminate cells expressing CD45 may be employed in cell depletion or killing. In one particularly preferred embodiment of the invention though, the present invention may be used to bring about cell death (preferably apoptosis) of CD45 expressing cells and hence depletion of such cells. In particular, the invention may bring about cross-linking of CD45 and hence cell death (preferably apoptosis), preferably with the improved ability of the invention to bring about cross-linking of CD45 also leading to more cell death (preferably apoptosis).

In one embodiment, as part of the cell transfer the subject may be given bone marrow as a way of transferring cells. In another embodiment, the subject may have been given cord blood, or cells isolated from cord blood, as a way of transferring cells. In another embodiment, the transplanted cells may have come from differentiated stem cells, for instance where stem cells have been differentiation in vitro and then transplanted.

In one embodiment where the invention is used to deplete or kill cells, a further cell depleting or killing agent may also be used as well. In a preferred embodiment, the binding molecule, in particular antibody, of the invention is the only cell depleting agent administered to the subject. In one embodiment, the level of depletion of the target cell is enough to be effective, for instance about at least 10%, 20%, 30%, 40%, 50%, 60%, 70%, 80%, 90% or 99% of the target cells. For example, on one embodiment, at least 50% of the target cells are depleted. In another embodiment, at least 75% of the target cells are depleted. In another embodiment, at least about 90% of the target cells are depleted. In another embodiment, at least about 95% of the target cells are depleted.

As discussed above, in a particularly preferred embodiment, the present invention may be used to bring about cell killing, in particular apoptosis, of a CD45 expressing cell. CD45 induced cell death (preferably apoptosis) may be identified by, for instance, one or more of cell shrinkage, membrane loosening, exteriorization of phosphatidic serine (PS) resides to the outer leaflet of the plasma membrane, reduction in mitochondrial transmembrane potential, and production of reactive oxygen species. Measurement or identification of those may, in one embodiment, be used to identify cell death (preferably apoptosis) of CD45 expressing cells. In one embodiment, cells stimulated to undergo cell death (preferably apoptosis) by the present invention may show one or more, and preferably all, of phosphatidylserine (PS) exposure (allowing Annexin V staining), membrane blebbing, retention of membrane integrity, nuclear condensation, and RNA/protein synthesis not being required for the cell death (preferably apoptosis) to happen. As the integrity of the cell is typically retained and PS is present on the external surface of the cell, staining, for instance with AnnexinV, may be used to identify cell death and in particular apoptosis of cells. Hence, in a preferred embodiment Annexin V staining is used to identify apoptotic cells. However, any suitable method may be used for assessing cell viability and hence cell killing.

In another embodiment, the present invention may be used in respect of graft-versus-host disease (GVHD). In one embodiment the GVHD is acute. In another embodiment, the GVHD is chronic. The invention may be used, for instance, to avoid the development of GVHD or to ameliorate the GVHD so it is of reduced severity. For example, the present invention may be used to deplete and/or kill target cells in cells, tissue, or organs to be transplanted prior to transplantation to a subject. In one embodiment, the invention therefore provides an ex vivo method of treating a cell population, tissue, or organ with a binding molecule, in particular an antibody, to deplete cells.

In another embodiment, the present invention provides a method of treatment comprising first performing such ex vivo treatment and then performing the transplantation. In another embodiment, the present invention is used to deplete or kill cells in a subject prior to the transplantation, so that there are fewer host cells able to attack the transplanted material as a way of reducing the chance of GVHD. Hence, the present invention also provides a method for treating or preventing GVHD comprising administering a binding molecule, in particular an antibody, of the present invention to deplete cells in a population of cells, tissue, or an organ prior to transplanting the population of cells, tissue, or an organ. The method may further comprise the transplantation itself. The depleted cells and transplanted cells, tissue, or organ, may be any of those mentioned herein. In one preferred embodiment, the transplanted cells are haematopoietic stem cells. In one preferred embodiment the depleted cells are T cells. In another preferred embodiment, the ability of the present invention to treat or prevent GVHD is employed in heart, lung, kidney, or liver transplants.

In another embodiment, the invention provides a method of depleting and/or killing cells in a population of cells, tissue, or organ prior to their transplantation, rather than treating the recipient. Hence, the present invention also provides a method of removing target cells from a population of cells, tissue, or organ prior to transplantation comprising treating the population of cells, tissue, or organ prior to transplantation and then performing the transplantation.

In one embodiment, the present invention may be used to deplete immune cells in organs or tissues, particularly where conventional therapies are unable to access readily or will lead to exaggerated inflammation as part of their inherent mechanism. In one embodiment, the present invention is used to deplete cells in an enclosed organ, for instance in the brain, spinal cord, eye or testes. In one embodiment, the invention may be employed to deplete CD45⁺ cells in immune privileged organs. The ability of the binding molecules of the invention to deplete CD45+ cells without employing Fc mediated functions may help avoid unwanted side-effects and damage. In one embodiment, the invention may be used to deplete cells immunosilently and without the need for antibody effector mechanisms. That may have the advantage of minimising, or at least reducing, unwanted damaged, for instance as enclosed organs can contain delicate and often non-dividing tissue cells that can be destroyed by infiltrating leukocytes. When the current invention is applied directly to the organ, for example the brain, spinal cord, eye or testes, it may result in the elimination of CD45 positive cells without inducing further damage or inflammation to the tissue or with reduced further damage. In one embodiment, the target cells in the enclosed organ are selected from lymphocytes, B cells, and T cells. In one embodiment, the target cells in the enclosed organ are, or comprise, CD4+ T cells. In another embodiment, the target cells are, or comprise, CD8+ T cells.

In a further preferred embodiment, the condition to be treated via the invention may be selected from one of the following:
viral encephalitis;
Glaucoma, particularly glaucoma characterised by T cell infiltration into the retina;
Parkinson's Disease;
ALS;
Paraneoplastic syndromes with CNS involvement;
Neuromyelitis Optica;
Autoimmune encephalitis;
Autoimmune uveitis; and
Chronic/autoimmune orchitis or other diseases of the testes that lead to infertility

In a further preferred embodiment, the condition the invention is applied to is one characterised by infiltrating CD8+ T cells.

### Pharmaceutical Compositions

In one aspect a pharmaceutical composition comprising: (a) a binding molecule or molecules, a nucleic acid molecule or molecules, or a vector or vectors of the present invention; and (b) a pharmaceutically acceptable carrier or diluent. In one preferred embodiment, the pharmaceutical composition comprises a binding molecule or molecules of the present invention. In one aspect, there is provided a pharmaceutical composition comprising one or more antibody of the present invention. In an especially preferred embodiment, it comprises an antibody or antibodies of the present invention. Various different components can be included in the composition, including pharmaceutically acceptable carriers, excipients and/or diluents. The composition may, optionally, comprise further molecules capable of altering the characteristics of the molecule(s) of the invention thereby, for example, reducing, stabilizing, delaying, modulating and/or activating the function of the molecule(s). The composition may be in solid, or liquid form and may be, inter alia, be in the form of a powder, a tablet, a solution or an aerosol.

The present invention also provides a pharmaceutical or diagnostic composition comprising a molecule of the present invention in combination with one or more of a pharmaceutically acceptable excipient, diluent or carrier. Accordingly, provided is the use of a binding molecule, in particular an antibody, of the invention for use in the treatment of, and for the manufacture of a medicament for the treatment of, a pathological condition or disorder. In one embodiment where the therapeutic of the invention is administered to a subject who is also being given a second therapeutic agent, the two may be given, for example, simultaneously, sequentially or separately. In one embodiment, the two are given in the same pharmaceutical composition. In another embodiment, the two are given is separate pharmaceutical compositions. In one embodiment, the present invention provides a binding molecule, in particular an antibody, of the invention for use in a method where the subject is also being treated with a second therapeutic agent. In another embodiment, the present invention provides the second therapeutic agent for use in a method where the subject is being treated with a binding molecule, in particular an antibody, of the present invention. The nucleic acid molecule(s) and vector(s) of the present invention may also be administered in such combinations.

A composition of the present invention will usually be supplied as a sterile, pharmaceutical composition. A pharmaceutical composition of the present invention may additionally comprise a pharmaceutically-acceptable adjuvant. In another embodiment, no such adjuvant is present in a composition of the present invention. The present invention also provides a process for preparation of a pharmaceutical or diagnostic composition comprising adding and mixing the binding molecule, in particular antibody, of the present invention together with one or more of a pharmaceutically acceptable excipient, diluent or carrier.

The term “pharmaceutically acceptable excipient” as used herein refers to a pharmaceutically acceptable formulation carrier, solution or additive to enhance the desired characteristics of the compositions of the present invention. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent solution used for the formulation, aseptic suspension of the antibody in the sterile buffered solvent solution, and dispensing of the formulation into sterile receptacles by methods familiar to those of ordinary skill in the art.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates. Pharmaceutically acceptable carriers in therapeutic compositions may additionally contain liquids such as water, saline, glycerol and ethanol. Such carriers enable the pharmaceutical compositions to be formulated as tablets, pills, dragées, capsules, liquids, gels, syrups, slurries and suspensions, for ingestion by the patient.

A thorough discussion of pharmaceutically acceptable carriers is available in Remington's Pharmaceutical Sciences (Mack Publishing Company, N.J. 1991).

The term “therapeutically effective amount” as used herein refers to an amount of a therapeutic agent needed to treat, ameliorate or prevent a targeted disease or condition, or to exhibit a detectable therapeutic or preventative effect. For any binding molecule, in particular antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 50 mg/kg, for example 0.1 mg/kg to 20 mg/kg per day. Alternatively, the dose may be 1 to 500 mg per day, such as 10 to 100, 200, 300 or 400 mg per day. Pharmaceutical compositions may be conveniently presented in unit dose forms containing a predetermined amount of an active agent of the invention. In one embodiment, the amount in a given dose is at least enough to bring about a particular function.

Compositions may be administered individually to a patient or may be administered in combination (e.g. simultaneously, sequentially or separately) with other agents, drugs or hormones. The dose at which the present invention is administered depends on the nature of the condition to be treated, the extent of the inflammation present and on whether the binding molecule, in particular the antibody, is being used prophylactically or to treat an existing condition.

The frequency of dose may depend on the half-life of the binding molecule, in particular of the antibody, and the duration of its effect. If it has a short half-life (e.g. 2 to 10 hours) it may be necessary to give one or more doses per day. Alternatively, if it has a long half-life (e.g. 2 to 15 days) it may only be necessary to give a dosage once per day, once per week or even once every 1 or 2 months. In some embodiments, it may be desirable for the therapeutic of the invention to be cleared from the system quickly after it has had its desired effect and so the binding molecule, in particular an antibody, of the present invention, may be deliberately chosen to therefore have a short half-life. For instance, in an embodiment of the invention where the aim is to deplete a target cell and that transfer cells to the subject, if the binding molecule, in particular antibody, of the present invention employed would also target the transferred cells it may be desirable for the binding molecule, in particular antibody, employed to have a short half-life to avoid it also targeting the transferred cells. That may mean, for instance, there can be less of a gap between the depletion of cells and the transfer of new cells.

In the present invention, the pH of the final formulation is not similar to the value of the isoelectric point of the binding molecule, in particular antibody, of the invention for if the pH of the formulation is 7 then a pI of from 8-9 or above may be appropriate. Whilst not wishing to be bound by theory it is thought that this may ultimately provide a final formulation with improved stability, for example the binding molecule, in particular antibody, remains in solution.

The binding molecules, in particular antibodies, and pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Direct delivery of the compositions will generally be accomplished by injection, subcutaneously, intraperitoneally, intravenously or intramuscularly, or delivered to the interstitial space of a tissue. The compositions can also be administered into a specific tissue of interest. Dosage treatment may be a single dose schedule or a multiple dose schedule. Where the product is for injection or infusion, it may take the form of a suspension, solution or emulsion in an oily or aqueous vehicle and it may contain formulatory agents, such as suspending, preservative, stabilising and/or dispersing agents. Alternatively, the binding molecule, in particular the antibody, may be in dry form, for reconstitution before use with an appropriate sterile liquid. If the composition is to be administered by a route using the gastrointestinal tract, the composition will need to contain agents which protect the antibody from degradation but which release the bispecific protein complex once it has been absorbed from the gastrointestinal tract.

A nebulisable formulation according to the present invention may be provided, for example, as single dose units (e.g., sealed plastic containers or vials) packed in foil envelopes. Each vial contains a unit dose in a volume, e.g., 2 ml, of solvent/solution buffer.

The present invention also provides a process for preparation of a pharmaceutical or diagnostic composition comprising adding and mixing a binding molecule of the present invention, in particular an antibody, together with one or more of a pharmaceutically acceptable excipient, diluent or carrier.

The binding molecule, in particular antibody, nucleic acid molecule, or vector may be the sole active ingredient in the pharmaceutical or diagnostic composition or may be accompanied by other active ingredients including antibody ingredients or non-antibody ingredients such as steroids or other drug molecules.

The pharmaceutical compositions suitably comprise a therapeutically effective amount of the binding molecule, in particular antibody, of the invention. The term “therapeutically effective amount” as used herein refers to an amount of a therapeutic agent needed to treat, ameliorate or prevent a targeted disease or condition, or to exhibit a detectable therapeutic or preventative effect. A “therapeutically effective amount” may be the amount required to bring about the desired level of cell depletion. For any binding molecule, in particular antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

Pharmaceutical compositions may be conveniently presented in unit dose forms containing a predetermined amount of an active agent of the invention per dose. A pharmaceutical composition of the present invention may be provided in a receptacle that provides means for administration to a subject. A pharmaceutical composition of the present invention may be provided in a prefilled syringe. The present invention therefore provides such a loaded syringe. It also provides an auto-injector loaded with a pharmaceutical composition of the present invention.

Compositions may be administered individually to a patient or may be administered in combination (e.g. simultaneously, sequentially or separately) with other agents, drugs or hormones.

Agents as employed herein refers to an entity which when administered has a physiological affect. Drug as employed herein refers to a chemical entity which at a therapeutic dose has an appropriate physiological affect.

The dose at which the molecule or molecules of the present invention are administered depends on the nature of the condition to be treated, the extent of the inflammation present and on whether the invention is being used prophylactically or to treat an existing condition. The frequency of dose will depend on the half-life of the binding molecule, in particular the antibody, and the duration of its effect. If the binding molecule, in particular of the antibody, has a short half-life (e.g. 2 to 10 hours) it may be necessary to give one or more doses per day. Alternatively, if the binding molecule, in particular antibody, has a long half-life (e.g. 2 to 15 days) and/or long lasting pharmacodynamics (PD) profile it may only be necessary to give a dosage once per day, once per week or even once every 1 or 2 months. In one embodiment the dose is delivered bi-weekly, i.e. twice a month.

In one embodiment, a single dose is administered. In one embodiment, a method of the present invention comprises administration until a target cell population is depleted and then stopping all administration. The method may comprise allowing the subject a break from administration prior to giving a cell transplant to the subject to give time for the antibody to clear from the system of the subject. In one embodiment doses are spaced to allow anti-drug (in this case anti-antibody) responses to wane before administration of further dose.

Half-life as employed herein is intended to refer to the duration of the molecule in circulation, for example in serum/plasma. Pharmacodynamics as employed herein refers to the profile and in particular duration of the biological action of the molecule according the present invention.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates. Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

Suitably in formulations according to the present invention, the pH of the final formulation is not similar to the value of the isoelectric point of the antibody or fragment, for example if the pI of the protein is in the range 8-9 or above then a formulation pH of 7 may be appropriate. Whilst not wishing to be bound by theory it is thought that this may ultimately provide a final formulation with improved stability, for example the binding molecule, in particular antibody, remains in solution. In one example the pharmaceutical formulation at a pH in the range of 4.0 to 7.0 comprises: 1 to 200 mg/mL of a binding molecule, in particular an antibody, according to the present invention, 1 to 100 mM of a buffer, 0.001 to 1% of a surfactant, a) 10 to 500 mM of a stabiliser, b) 10 to 500 mM of a stabiliser and 5 to 500 mM of a tonicity agent, or c) 5 to 500 mM of a tonicity agent.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. It will be appreciated that the active ingredient in the composition will be an antibody molecule. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract. A composition of the present invention may be in one embodiment injected into an enclosed organ, for instance any of those mentioned herein.

A thorough discussion of pharmaceutically acceptable carriers is available in Remington's Pharmaceutical Sciences (Mack Publishing Company, N.J. 1991).

In one embodiment the formulation is provided as a formulation for topical administrations including inhalation. Suitable inhalable preparations include inhalable powders, metering aerosols containing propellant gases or inhalable solutions free from propellant gases. Inhalable powders according to the invention containing the active substance may consist solely of the abovementioned active substances or of a mixture of the abovementioned active substances with physiologically acceptable excipient. These inhalable powders may include monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 1 to 5 μm. The particle size of the active ingredient (such as the antibody or fragment) is of primary importance.

The propellant gases which can be used to prepare the inhalable aerosols are known in the art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellent gases may be used on their own or in mixtures thereof.

Particularly suitable propellent gases are halogenated alkane derivatives selected from among TG 11, TG 12, TG 134a and TG227. Of the abovementioned halogenated hydrocarbons, TG134a (1,1,1,2-tetrafluoroethane) and TG227 (1,1,1,2,3,3,3-heptafluoropropane) and mixtures thereof are particularly suitable.

The propellent-gas-containing inhalable aerosols may also contain other ingredients such as cosolvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active ingredient.

Alternatively topical administrations to the lung may also be by administration of a liquid solution or suspension formulation, for example employing a device such as a nebulizer, for example, a nebulizer connected to a compressor (e.g., the Pari LC-Jet Plus® nebulizer connected to a Pari Master® compressor manufactured by Pari Respiratory Equipment, Inc., Richmond, Va.).

The binding molecule, in particular antibody, of the invention can be delivered dispersed in a solvent, e.g., in the form of a solution or a suspension. It can be suspended in an appropriate physiological solution, e.g., saline or other pharmacologically acceptable solvent or a buffered solution. A suspension can employ, for example, lyophilised binding molecule, in particular lyophilised antibody.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes. This may include production and sterilization by filtration of the buffered solvent/solution used for the formulation, aseptic suspension of the antibody in the sterile buffered solvent solution, and dispensing of the formulation into sterile receptacles by methods familiar to those of ordinary skill in the art.

Nebulizable formulation according to the present invention may be provided, for example, as single dose units (e.g., sealed plastic containers or vials) packed in foil envelopes. Each vial contains a unit dose in a volume, e.g., 2 mL, of solvent/solution buffer.

The invention may be suitable for delivery via nebulisation.

The present invention also provides a syringe loaded with a composition comprising a binding molecule, in particular an antibody, of the invention. In one embodiment, a pre-filled syringe loaded with a unit dose of a binding molecule, in particular of an antibody of the invention, is provided. In another embodiment, an auto injector loaded with binding molecule, in particular an antibody, of the invention is provided. In a further embodiment, an IV bag loaded with binding molecule, in particular an antibody, of the invention is provided. Also provided, is the binding molecule, in particular antibody of the invention in lyophilised form in a vial or similar receptacle in lyophilized form.

It is also envisaged that the binding molecule, in particular antibody, of the present invention may be administered by use of gene therapy. In order to achieve this, where the binding molecule is an antibody, DNA sequences encoding the heavy and light chains of the antibody molecule under the control of appropriate DNA components are introduced into a patient such that the antibody chains are expressed from the DNA sequences and assembled in situ.

In one embodiment, the binding molecule, in particular antibody, of the present invention may be used to functionally alter the activity of the antigen or antigens of interest and in particular to modulate CD45. For example, the invention may neutralize, antagonize or agonise the activity of said antigen or antigens, directly or indirectly.

The present invention also extends to a kit, comprising a binding molecule, in particular an antibody, of the invention. In one embodiment a kit comprising any of the binding molecules, in particular antibodies, of the invention is provided, optionally with instructions for administration.

In yet another embodiment, the kit further comprises one or more reagents for performing one or more functional assays.

In one embodiment, molecules of the present invention including an antibody of the invention is provided for use as a laboratory reagent.

### Further Aspects

In a further aspect, there is provided a nucleotide sequence, for example a DNA sequence encoding an antibody molecule of the present invention as described herein. In one embodiment, there is provided a nucleotide sequence, for example a DNA sequence encoding a binding molecule, in particular an antibody, of the present invention as described herein. In one embodiment, the nucleotide sequence is collectively present on more than one polynucleotide but collectively together they are able to encode a binding molecule, in particular an antibody, of the present invention.

The invention herein also extends to a vector comprising a nucleotide sequence as defined above. The term “vector” as used herein refers to a nucleic acid molecule capable of transporting another nucleic acid to which it has been linked. An example of a vector is a “plasmid,” which is a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell, where they are subsequently replicated along with the host genome. In the present specification, the terms “plasmid” and “vector” may be used interchangeably as a plasmid is the most commonly used form of vector. General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

The term vector herein also includes, for example, particles comprising the vector, for example LNP (Lipid Nanoparticle) particles and in particular LNP-mRNA particles. It also includes viral particles used for transferring a vector of the present invention.

The term “selectable marker” as used herein refers to a protein whose expression allows one to identify cells that have been transformed or transfected with a vector containing the marker gene. A wide range of selection markers are known in the art. For example, typically the selectable marker gene confers resistance to drugs, such as G418, hygromycin or methotrexate, on a host cell into which the vector has been introduced. The selectable marker can also be a visually identifiable marker such as a fluorescent marker for example. Examples of fluorescent markers include rhodamine, FITC, TRITC, Alexa Fluors and various conjugates thereof.

In one embodiment, the invention provides a vector encoding a binding molecule, in particular an antibody, of the invention. In another embodiment, the invention provides vectors which collectively encode a binding molecule, in particular an antibody, of the invention.

Also provided is a host cell comprising one or more cloning or expression vectors comprising one or more DNA sequences encoding an antibody of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells. A host cell comprising a nucleic acid molecule or vector of the present invention is also provided.

The present invention also provides a process for the production of a molecule according to the present invention or a component thereof comprising culturing a host cell containing a vector of the present invention under conditions suitable for leading to expression of protein from DNA encoding the molecule of the present invention, and isolating the molecule.

A method for producing an antibody which comprises a heterodimeric tether may further comprise mixing the two parts of the antibody and allowing the binding partners of the heterodimeric tether to associate. The method may further comprise purification, for example to remove any species apart from the desired heterodimers.

The binding molecules, in particular antibodies, of the present invention may be used in diagnosis/detection kits. In one embodiment, antibodies of the present invention are fixed on a solid surface. The solid surface may for example be a chip, or an ELISA plate.

The binding molecules, in particular antibodies, of the present invention may be for example be conjugated to a fluorescent marker which facilitates the detection of bound antibody-antigen complexes. They can be used for immunofluorescence microscopy. Alternatively, the binding molecule, in particular antibody, may also be used for western blotting or ELISA.

In one embodiment, there is provided a process for purifying a binding molecule, in particular an antibody, of the present invention or a component thereof. In one embodiment, there is provided a process for purifying a binding molecule, in particular an antibody, according the present invention or a component thereof comprising the steps: performing anion exchange chromatography in non-binding mode such that the impurities are retained on the column and the antibody is maintained in the unbound fraction. The step may, for example be performed at a pH about 6-8. The process may further comprise an initial capture step employing cation exchange chromatography, performed for example at a pH of about 4 to 5. The process may further comprise of additional chromatography step(s) to ensure product and process related impurities are appropriately resolved from the product stream. The purification process may also comprise of one or more ultra-filtration steps, such as a concentration and diafiltration step.

“Purified form” as used supra is intended to refer to at least 90% purity, such as 91, 92, 93, 94, 95, 96, 97, 98, 99% w/w or more pure.

In the context of this specification “comprising” is to be interpreted as “including”. Aspects of the invention comprising certain elements are also intended to extend to alternative embodiments “consisting” or “consisting essentially” of the relevant elements.

Positively recited embodiments may be employed herein as a basis for a disclaimer.

Where the singular is referred to herein, the plural is also encompassed unless otherwise stated or apparent. In particular, The singular forms “a,”, “an”, “the” and the like include plural referents unless the context clearly dictates otherwise.

All references referred to herein are specifically incorporated by reference.

The sub-headings herein are employed to assist in structuring the specification and are not intended to be used to construct the meaning of technical terms herein.

Sequences of the invention are provided herein below.

In the context of this specification “comprising” is to be interpreted as “including”. Aspects of the invention comprising certain elements are also intended to extend to alternative embodiments “consisting” or “consisting essentially” of the relevant elements. Positively recited embodiments may be employed herein as a basis for a disclaimer.
All references referred to herein are specifically incorporated by reference

### REFERENCES

1. Ribosome display efficiently selects and evolves high-affinity antibodies in vitro from immune libraries. Hanes J, Jermutus L, Weber-Bornhauser S, Bosshard H R, Plückthun A. (1998) Proc. Natl. Acad. Sci. U.S.A. 95, 14130-14135
2. Directed in Vitro Evolution and Crystallographic Analysis of a Peptide-binding Single Chain Antibody Fragment (scFv) with Low Picomolar Affinity. Zhand C, Spinelli S, Luginbuhl B, Amstutz P, Cambillau C, Pluckthun A. (2004) J. Biol. Chem. 279, 18870-18877
3. Antigen recognition by conformational selection. Berger C, Weber-Bornhauser S, Eggenberger Y, Hanes J, Pluckthun A, Bosshard H. R. (1999) F.E.B.S. Letters 450, 149-153

### EXAMPLES

The term Fab-X/-Fab-Y (or Fab-KD-Fab) as used in the Examples describes a protein complex format having the formula A-X:Y-B wherein:
A-X is a first fusion protein;
Y-B is a second fusion protein;
X:Y is a heterodimeric-tether;
A comprises a Fab fragment specific to an antigen such as CD45;
B comprises a Fab fragment specific to an antigen such as CD45;
X is a first binding partner of a binding pair such as a scFv (e.g. a scFv that binds GCN 4 peptide);
Y is a second binding partner of the binding pair such as a peptide (e.g a GCN peptide);
: is an interaction (such as a binding interaction) between X and Y;
is a bond or a linker.

A number of the antibody molecules used in the Examples are in such a format. Others are in the BYbe format. BYbe antibodies used in the Examples are in the following format:
c) a polypeptide chain: V_{H}CH₁-ScFv;
d) a polypeptide chain: V_{L}C_{L};
wherein:
the V_{H}CH₁ and V_{L}C_{L} pair with each other to form a Fab;
the CH₁ and ScFv are joined by a bond or a linker “-”.

### Example 1—Production of Fab-X (Fab-52SR4 scFv) and Fab-Y (Fab-GCN4 Peptide) for Functional Assays

### Method

### Cloning Strategy

Antibody variable region DNA was generated by PCR or gene synthesis and contained flanking restriction enzyme sites. These sites were HindIII and XhoI for variable heavy chains and HindIII and BsiWI for variable light chains. This makes the heavy variable region amenable to ligating into the two heavy chain vectors (pNAFH with Fab-Y and pNAFH with Fab-X ds [disulphide stabilised]) as they have complementary restriction sites. This ligates the variable region upstream (or 5′) to the murine constant regions and peptide Y (GCN4) or scFv X (52SR4) creating a whole reading frame. The light chains were cloned into standard in house murine constant kappa vectors (pMmCK or pMmCK S171C) which again use the same complimentary restriction sites. The pMmCK S171C vector is used if the variable region is isolated from a rabbit. The cloning events were confirmed by sequencing using primers which flank the whole open reading frame.

### Cultivating CHOS

Suspension CHOS cells were pre-adapted to CDCHO media (Invitrogen) supplemented with 2 mM (100×) glutamax. Cells were maintained in logarithmic growth phase agitated at 140 rpm on a shaker incubator (Kuner AG, Birsfelden, Switzerland) and cultured at 37° C. supplemented with 8% CO₂.

### Electroporation Transfection

Prior to transfection, the cell numbers and viability were determined using CEDEX cell counter (Innovatis AG. Bielefeld, Germany) and required amount of cells (2×10⁸ cells/ml) were transferred into centrifuge conical tubes and were spun at 1400 rpm for 10 minutes. The Pelleted cells were re-suspended in sterile Earls Balanced Salts Solution and spun at 1400 rpm for further 10 minutes. Supernatant was aspirated and pellets were re-suspended to desired cell density.

Vector DNA at a final concentration of 400 μg for 2×10⁸ cells/ml mix and 800 μl was pipetted into Cuvettes (Biorad) and electroporated using in-house electroporation system. Transfected cells were transferred directly into one 3 L Erlenmeyer Flasks containing ProCHOS media enriched with 2 mM glutamx and antibiotic antimitotic (100×) solution (1 in 500) and Cells were cultured in Kuhner shaker incubator set at 37° C., 5% CO₂ and 140 rpm shaking. Feed supplement 2 g/L ASF (AJINOMOTO) was added at 24 hr post transfection and temperature dropped to 32° C. for further 13 days culture. At day four 3 mM Sodium buryrate (n-BUTRIC ACID Sodium Salt, Sigma B-5887) was added to the culture. On day 14, cultures were transferred to tubes and supernatant separated from the cells after centrifugation for 30 minutes at 4000 rpm. Retained supernatants were further filtered through 0.22 μm SARTO BRAN P Millipore followed by 0.22 μm Gamma gold filters. Final expression levels were determined by Protein G-HPLC.

### Large Scale (1.0 L) Purification

Fab-X and Fab-Y were purified by affinity capture using the AKTA Xpress systems and HisTrap Excel pre-packed nickel columns (GE Healthcare). The culture supernatants were 0.22 μm sterile filtered and pH adjusted to neutral, if necessary, with weak acid or base before loading onto the columns. A secondary wash step, containing 15-25 mM Imidazole, was used to displace any weakly bound host cell proteins/non-specific His binders from the nickel resin. Elution was performed with 10 mM sodium phosphate, pH 7.4+1 M NaCl+250 mM Imidazole and 2 ml fractions collected. One column volume into the elution the system was paused for 10 minutes to tighten the elution peak, and consequently decrease the total elution volume. The cleanest fractions were pooled and buffer exchanged into PBS (Sigma), pH 7.4 and 0.22 μm filtered. Final pools were assayed by A280 Scan, SE-HPLC (G3000 method), SDS-PAGE (reduced & non-reduced) and for endotoxin using the Endosafe nexgen-PTS system (Charles River).

### Example 2—Production of Fabs and BYbes

### Method

To generate Fab fragments of anti-CD45 antibodies 4133 and 6294, genes encoding their respective light and heavy chain V-regions were designed and constructed by an automated synthesis approach (ATUM). The V-region genes of rabbit antibody 4133 were cloned into expression vectors containing DNA encoding rabbit Cκ1 region and heavy chain γ CH1 region, respectively. The V-regions genes of mouse antibody 6294 were cloned into expression vectors containing DNA encoding mouse Cκ region and heavy chain γ1 CH1 region, respectively.

Similarly, the full length of the heavy chains (Fab HC-G4S linker-scFv) of 4133-6294 and NegCtrl BYbes were designed and constructed by an automated synthesis approach (ATUM). Both heavy chains were cloned into in-house mammalian expression vectors. The 4133-6294 BYbe heavy chain was paired with the 4133 light chain described above. The light chain V-region gene of the NegCtrl BYbe, was designed and constructed by an automated synthesis approach (ATUM), and then cloned into expression vectors containing DNA encoding mouse Cκ region. NegCtrl BYbe has antigen-irrelevant specificity in both the Fab and scFv positions.

The relevant heavy and light chain constructs were paired and transfected into CHO-SXE cells using Gibco ExpiFectamine CHO Transfection Kit (cat no. A29133, ThermoFisher Scientific) according to manufacturer's instructions. The cells were cultured for 7 days in an incubator at 37° C., 5% CO₂ with 140 rpm shaking. Following the incubation, cultures were transferred to tubes and supernatant separated from the cells after centrifugation for 30 minutes at 4000 rpm. Retained supernatants were filtered through 0.22 μm SARTO BRAN P Millipore followed by 0.22 μm Gamma gold filters.

All proteins from the supernatants were purified using two 5 ml HiTrap Protein G HP columns (cat no. GE29-0405-01, SigmaAldrich) in series on an AKTA Pure purification system (GE Healthcare Life Sciences) according to manufacturer's instructions. The fractions of eluted protein were combined and concentrated to <5 ml with Amicon Ultra-15 centrifugal filter unit with Ultracel-10 membrane 10 kDa (cat no. UFC9010, SigmaAldrich).

To obtain a clean fraction, the proteins were then passed through a HiLoad Superdex 200 pg 16/60 HPLC size exclusion column on an AKTA Pure purification system. Protein concentration was determined using a Thermo Scientific NanoDrop 2000 (cat no. ND-2000). Additionally, fractions were analysed on a 4-20% Tris-glycine gel and tested for endotoxin using the Endosafe nexgen-MCS system (Charles River).

### Example 3—Production of CD45 Extracellular Domain

### Method

A gene encoding domains 1˜4 of the extracellular domain of CD45 (UniProtKB—P08575, residue positions 225-573) was designed and constructed by an automated synthesis approach (ATUM). To aid purification, a TEV cleavage site and a 10-His tag was incorporated at C-terminus of the expressed protein. The gene was cloned into an in-house mammalian expression vector and then transfected into HEK293 cells using Gibco ExpiFectamine 293 Transfection Kit (cat no. A14525, ThermoFisher Scientific) according to manufacturer's instructions. The cells were cultured for 7 days in an incubator at 37° C., 5% CO₂ with 140 rpm shaking. Following the incubation, cultures were transferred to tubes and supernatant separated from the cells after centrifugation for 30 minutes at 4000 rpm. Retained supernatants were filtered through 0.22 μm SARTO BRAN P Millipore followed by 0.22 μm Gamma gold filters.

The His-tagged protein from the supernatant was purified using two 1 ml HisTrap Excel columns (cat no. GE17-3712-05, SigmaAldrich) in series on an AKTA Pure purification system (GE Healthcare Life Sciences) according to manufacturer's instructions. The fractions of eluted protein were combined and concentrated to <5 ml with Amicon Ultra-centrifugal filter unit with Ultracel-3 membrane 3 kDa (cat no. UFC900308, SigmaAldrich). To obtain a clean fraction, the protein was then passed through a HiLoad Superdex 75 pg 16/60 HPLC size exclusion column on an AKTA Pure purification system. Protein concentration was determined using a Thermo Scientific NanoDrop 2000 (cat no. ND-2000). Additionally, fractions were analysed on a 4-20% Tris-glycine gel.

### Example 4—Anti-CD45 Fab-KD-Fab Induces Apoptosis as Determined by Annexin V Binding

### Method

Human PBMC derived from blood leukocyte platelet-apheresis cones (NHSBT Oxford) were banked as frozen aliquots. Prior to an assay being performed, 2 vials per donor cone of frozen cells, each containing 5×10⁷ cells in 1 ml, were thawed in a 37° C. water bath and then added to 50 ml complete media (RPMI 1640+2 mM GlutaMAX+1% Pen/Strep, all previous supplied by Invitrogen, +10% Fetal Bovine Serum (FBS), Sigma Aldrich). Cells were spun (500 g, 5 min, at RT) and re-suspended in 30 ml complete media to wash and spun again. Cells were resuspended in 20 ml complete media, counted and counted on a ChemoMetec NucleoCounter NC-3000 to determine concentration and viability, and then diluted to 1.25×10⁶ cells/ml. 10⁵ cells per well in 80 μl were then added to each well of a Corning Costar 96-well, cell culture treated, U-shaped-bottom microplate (cat no. 07-200-95) and rested in a 37° C., 5% CO₂ incubator for 2 hrs. PBMCs from three donors, UCB-Cones 652, 658 and 686 were used in this assay.

Fab-KD-Fab reagents can form non-covalently linked Fab-Fab combinations by premixing two separate halves, labelled X and Y. In a Greiner 96-well non-binding microplate, Fab-X and Fab-Y combinations NegCtrl-X/4133-Y, 6294-X/NegCtrl-Y, 6294-X/4133-Y, and 6294-X/6294-Y, were added to complete media to give a Fab-KD-Fab concentration of 500 nM. The microplate was incubated for 1 hr at 37° C., 5% CO₂. NegCtrl-X and NegCtrl-Y are negative controls which are specific for an irrelevant antigen.

20 μl of each Fab-KD-Fab preparation was then added to the cells (final Fab-KD-Fab concentration of 100 nM) and incubated for 24 hrs at 37° C., 5% CO₂. Following the incubation, plates were spun at 500 g for 5 min at RT and the media aspirated using a BioTek ELx405 microplate washer (20 μl U bottom aspirate setting), to leave the cells in 20 μl residual media.

Multicyt apoptosis kit (Intellicyt cat no. 90054) was used according to the manufacturer's instructions. A 2×working concentration of staining cocktail was prepared in complete media. 20 μl of the antibody staining cocktail was added to the cells and the plate incubated for 1 hr at 37° C., 5% CO₂. Live cells were analysed using the Intellicyt iQue Screener PLUS. Live cell counts were extracted as metrics and graphical representations generated using Graphpad Prism version 8.1 (Graphpad).

### Results

Data from a representative donor (UCB Cone-686) is shown FIG. 1(A) & 1(B). (A) A marked reduction in lymphocyte cell count was observed in 6294-X/4133-Y-treated cells compared with those treated with NegCtrl-X/4133-Y, 6294-X/NegCtrl-Y, 6294-X/6294-Y or left untreated. (B) Of the surviving cells in 6294-X/4133-Y wells, 38% showed annexin V binding. This is indicative of cells undergoing apoptosis. Furthermore, the level of annexin V binding was roughly 3-fold greater than that in other treated and untreated wells.

### Example 5—Apoptosis of Purified T Cells

### Method

Human PBMC derived from blood leukocyte platelet-apheresis cones (NHSBT Oxford) were banked as frozen aliquots. Prior to an assay being performed, 2 vials per donor cone of frozen cells, each containing 5×10⁷ cells in 1 ml, were thawed in a 37° C. water bath and then added to 50 ml RPMI media (RPMI 1640+2 mM glutamine+1% penicillin/streptomycin, supplied by Invitrogen, 5% Heat Inactivated human AB serum, cat no. H3667-20ML, Sigma Aldrich). Cells were spun (500 g, 5 min, at RT) and re-suspended in 30 ml complete media to wash and spun again. Cells were resuspended in 20 ml RPMI media, counted and counted on a ChemoMetec NucleoCounter NC-3000 to determine concentration and viability, and then diluted to 1.25×10⁶ cells/ml. 10⁵ cells per well in 80 μl were then added to each well of a Corning Costar 96-well, cell culture treated, U-shaped-bottom microplate (cat no. 07-200-95) and rested in a 37° C., 5% CO₂ incubator for 2 hrs.

T cells were purified using a CD4+ T cell Isolation Kit according to the manufacturer's instructions (cat no. 130-096-533, Miltenyi Biotec). Briefly, PBMCs were washed in cold MACS buffer (PBS pH 7.2, 0.5% bovine serum albumin and 2 mM EDTA, Sigma Aldrich) and resuspended at 10⁷ cells in 40 μl of MACS buffer. 10 μl of CD4+ T cell Biotin-Antibody cocktail was then added (per 10⁷ cells), mixed and then incubated for 5 min, at 4° C.). A further 30 μl of MACS buffer was then added (per 10⁷ cells) followed by 20 μl of a CD4+ T cell Microbead cocktail (per 10⁷ cells). Cells were mixed and then incubated for 10 min at 4° C. To separate CD4+ T cells from other cells they were placed on a magnetic selection column (LS column) and washed with three times with 3 ml of MACS buffer. The purified CD4+ T cells were collected from the column eluate. The cells were then washed in RPMI media (as above) and counted to assess recovery and viability (measured as 97% cell viability). 10⁵ cells per well in 100 μl were then added to each well of a Corning Costar 96-well, cell culture treated, U-shaped-bottom microplate (cat no. 07-200-95).

In a Greiner 96-well non-binding microplate, Fab-X and Fab-Y combinations 6294-X/4133-Y, 4133-Y/6294-Y, 6294-X/6294-Y and NegCtrl-X/4133-Y, were added to complete media to give a Fab-KD-Fab concentration of 200 nM. The microplate was incubated for 1 hr at 37° C., 5% CO₂. Following the incubation, Fab-KD-Fab reagents were then serially diluted in RPMI media 1 in 5, seven times to produce to form an 8-point dose curve. It should be noted that when two Y-reagents are added together (as here using the combination of 4133-Y and 6294-Y) these form a mixture and not a linked molecule.

100 μl of each Fab-KD-Fab or BYbe dilution (final well concentrations 100-0.00128 nM) was then added to the plates of CD4+ cells, and incubated for 24 hours at 37° C., 5% CO₂. Following the incubation, plates were spun at 500 g for 5 min at RT. The buffer was then aspirated (using a BioTek ELx405 microplate washer, 15 μl U bottom aspirate setting) plates sealed and re-spun at 1800 rpm for 30 seconds. Plates were topped up with ice cold FACS buffer (PBS+1% BSA+0.1% NaN₃+2 mM EDTA) and spun again. Buffer was removed, the plates re-spun and 20 μl of 1:1000 near IR dye (Invitrogen) was added to each well. After 20 mins cells were washed in 200 μl of FACS buffer was added and resuspended in 15 μl of FACS buffer before being analysed using the Intellicyt iQue Screener PLUS. Live cell counts were extracted as metrics and graphical representations generated using Graphpad Prism version 8.1 (Graphpad). Asymmetric (five parameter) curve fitting was applied to derive maximal % cell reduction and EC50 values.

### Results

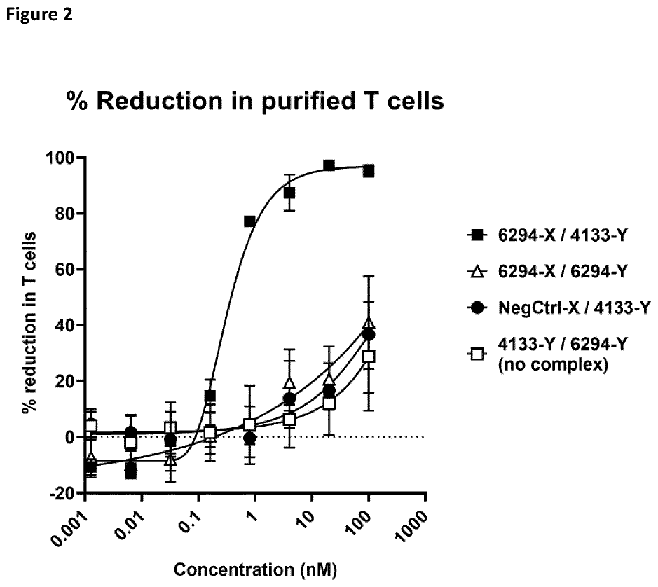

The percentage reduction in purified CD4+ T cell numbers is shown in FIG. 2. The combination 6294-X/4133-Y showed the highest level of reduction at 97% and was the most potent giving an EC50 value of 0.32 nM. All other combinations did not reach 50% maximal levels for cell reduction and were not-potent enough to generate EC50 readings.

### Example 6—Apoptosis of PBMCs Induced by Anti-CD45 Antibodies Formatted as Fab-X/Fab-Y and BYbe

### Method

Human PBMC derived from blood leukocyte platelet-apheresis cones (NHSBT Oxford) were banked as frozen aliquots. Prior to an assay being performed, 2 vials of frozen cells, each containing 5×10⁷ cells in 1 ml, were thawed in a 37° C. water bath and then added to 50 ml complete media (RPMI 1640+2 mM GlutaMAX+1% Pen/Strep, all previous supplied by Invitrogen, +10% Fetal Bovine Serum (FBS), Sigma Aldrich). Cells were spun (500 g, 5 min, at RT) and re-suspended in 30 ml complete media to wash and spun again. Cells were resuspended in 10 ml complete media and then counted using a ChemoMetec NucleoCounter NC-3000. 10⁵ cells per well in 100 μl were then added to each well of a Corning Costar 96-well, cell culture treated, U-shaped-bottom microplate (cat no. 07-200-95). The plate was rested in a 37° C., 5% CO₂ incubator for 2 hrs. PBMCs from two donors, UCB-Cones 801 and 802 were used in this assay.

In a Greiner 96-well non-binding microplate, Fab-X and Fab-Y combination 6294-X/4133-Y was added to complete media to give a Fab-KD-Fab concentration of 1500 nM. Similarly, a 1500 nM stock of 4133-6294 BYbe in complete media was prepared. The microplate was incubated for 1 hr at 37° C., 5% CO₂. Following the incubation, Fab-KD-Fab and BYbe reagents were then serially diluted in complete media 1 in 5, nine times to produce to form a 10-point dose curve.

20 μl of each Fab-KD-Fab or BYbe dilution (final well concentrations 250-0.000128 nM) was added to the cells and incubated for 24 hrs at 37° C., 5% CO₂. Following the incubation, the plate was spun at 500 g, 5 min, RT, the buffer was aspirated with a BioTek ELx405 microplate washer, and the cells were re-suspended in FACS buffer (PBS+1% bovine serum albumin (BSA)+0.1% NaN₃+2 mM EDTA, Sigma Aldrich) to wash and then re-spun and buffer was aspirated to leave the cells in 20 μl residual media. 20 μl of cell-specific marker antibody cocktail solution was added to the wells and incubated for 1 hr at 4° C. The antibody cocktail is detailed in Table 4 below.

Cells were analysed live using the Intellicyt iQue Screener PLUS. Live cell counts were extracted as metrics and graphical representations generated using Graphpad Prism version 8.1 (Graphpad). Asymmetric (five parameter) curve fitting was applied to derive maximal % cell reduction and EC50 values.

TABLE 4

| Cell-specific antibody cocktail | Supplier | Clone | Dilution |
|---|---|---|---|
| LIVE/DEAD ™ Fixable | Invitrogen | | 1:1000 |
| Near-IR Dead Cell | | | |
| Stain | BD Biosciences | UCH-L1 | 1:20 |
| Mouse Anti-Human CD45RO | BD Biosciences | H1-100 | 1:40 |
| PerCP-Cy5.5 | | | |
| Mouse Anti-Human | Biolegend | M5E2 | 1:20 |
| CD45 RA FITC | | | |
| Mouse Anti-Human CD14 BV570 | Biolegend | WM53 | 1:40 |
| Mouse Anti-Human CD33 BV785 | Biolegend | 6D5 | 1:40 |
| Mouse Anti-Human CD19 BV650 | BD Biosciences | B159 | 1:20 |
| Mouse Anti-Human CD56 PE | BD Biosciences | RPA-T8 | 1:20 |
| Mouse Anti-Human CD8 PE-Cy7 | Biolegend | SK3 | 1:20 |
| Mouse Anti-Human CD4 BV510 | Biolegend | 3C10 | 1:20 |
| Mouse Anti-Human Vα7.2 BV421 | BD Biosciences | GL3 | 1:20 |
| Mouse Anti-Human γδ TCR APC | | | |

### Results

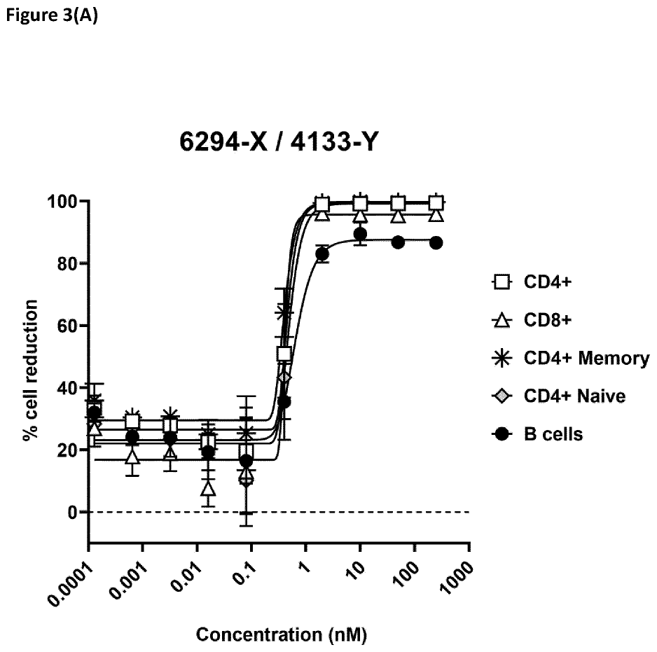

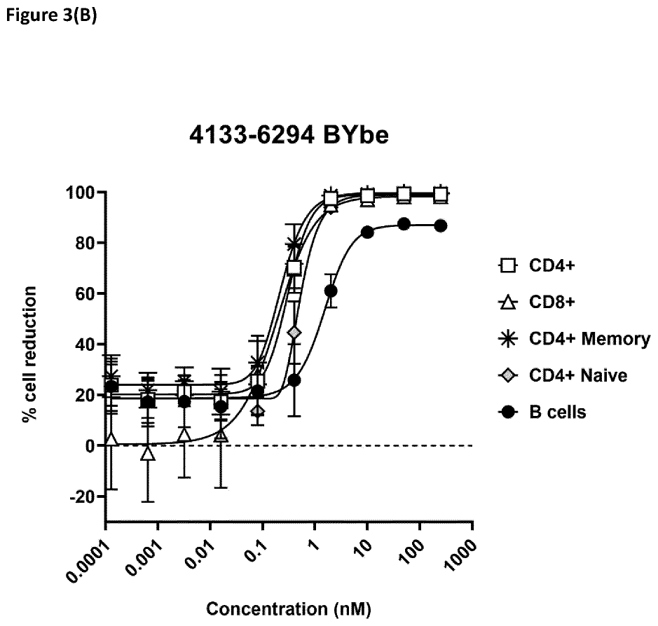

The percentage reductions in PBMC cell subset numbers by (A) 6294-X/4133-Y and (B) 4133-6294 BYbe for a representative donor (UCB Cone-802) are shown in FIG. 3 and Tables 5 and 6 below. Both 6294-X/4133-Y and 4133-6294 BYbe showed almost maximal reductions in T cells (>95%) and B cells (87%) with highly potent EC50's of 0.19-0.52 nM in T cells and 0.65-1.50 in B cells.

TABLE 5
Top and bottom levels of % subset cell reduction,
and EC50 (nM) values, for 6294-X/4133-Y.

| 6294-X/ | | | | CD4+ | CD4+ |
|---|---|---|---|---|---|
| 4133-Y | B cells | CD4+ | CD8+ | Memory | Naive |
| Top | 87.62 | ~99.28 | 95.69 | 99.74 | 99.24 |
| Bottom | 23.23 | 26.54 | 16.85 | 29.5 | 22.04 |
| EC50 (nM) | 0.6484 | 0.4594 | ~0.4133 | 0.4027 | 0.5054 |

TABLE 6
Top and bottom levels of % subset cell reduction,
and EC50 (nM) values, for 4133-6294 BYbe

| 4133-6294 | | | | CD4+ | CD4+ |
|---|---|---|---|---|---|
| BYbe | B cells | CD4+ | CD8+ | Memory | Naive |
| Top | 87.09 | 99.2 | 98.36 | 99.76 | 99.04 |
| Bottom | 18.74 | 20.22 | 0.543 | 24 | 18.53 |
| EC50 (nM) | 1.502 | 0.3021 | 0.1873 | 0.2222 | 0.5241 |

### Example 7—Apoptosis of Lymphocytes in Whole Blood

### Method

Human whole blood (Lithium heparin tubes) was taken from two donors (HTA #051119-01 & #051119-02) at UCB Pharma Slough, UK according to approved ethical sample collection protocol.

In a Greiner 96-well non-binding microplate, Fab-X and Fab-Y combinations 6294-X/4133-Y and NegCtrl-X/4133-Y, were added to PBS to give a Fab-KD-Fab concentration of 2750 nM. Similarly, a 2750 nM stock of 4133-6294 BYbe in PBS was prepared. The microplate was incubated for 1 hr at 37° C., 5% CO₂. Following the incubation, Fab-KD-Fab and BYbe reagents were then serially diluted in PBS 1 in 5, nine times to produce to form a 10-point dose curve.

5 μl of each Fab-KD-Fab or BYbe dilution was added to Nunc™ 96-Well Polypropylene DeepWell™ plates (ThermoFisher). Then 50 μl of blood was added to each well, the plate gently mixed and sealed with a plate seal that allows gas exchange. These cells were then incubated for 5 hrs at 37° C., 5% CO₂. Running the assay for a short time period avoids the need to add an anti-coagulant.

Following the incubation, 950 μl of BD Phosflow BD Lyse/Fix (cat no. BD558049, FisherScientific) was added to each well and the plate incubated at 37° C., 5% CO₂ for 10 minutes. The plate was then spun at 500 g for 8 min, at 4° C. The buffer was aspirated and 1 ml of FACS buffer (PBS+1% bovine serum albumin (BSA)+0.1% NaN₃+2 mM EDTA, Sigma Aldrich) added to wash cells using an Integra Viaflo 96 channel pipette. The plate was spun at 500 g for 8 min, at 4° C. As before, the buffer was aspirated and 1 ml of FACS buffer added to wash cells. This was followed by a slower spin at 250 g for 10 min, at 4° C. Again, buffer was aspirated and 1 ml of FACS buffer added to wash cells.

The plate was re-spun at 500 g for 8 min, at 4° C. Buffer was aspirated leaving the cells in a minimal residual volume for cell-specific antibody staining. 20 μl of cell-specific antibody cocktail (shown in Table 7 below) was added to the wells and the plate incubated for 1 hr at 4° C.

Following the incubation with the antibody cocktail, cells were washed twice as outlined above and buffer aspirated to leave the cells in 20 μl residual buffer. 20 μl of FACS buffer was then added to samples to dilute the cells. Cells were analysed live using the Intellicyt iQue Screener PLUS. Live cell counts were extracted as metrics and graphical representations generated using Graphpad Prism version 8.1 (Graphpad). Asymmetric (five parameter) curve fitting was applied to derive maximal % cell reduction and EC50 values.

TABLE 7

| Antibody/Reagent | Supplier | Clone | Dilution |
|---|---|---|---|
| LIVE/DEAD ™ Fixable | Invitrogen | | 1:1000 |
| Near-IR Dead Cell | | | |
| Stain | Invitrogen | UCH-L1 | 1:20 |
| Mouse Anti-Human | BD Biosciences | RPA-T4 | 1:20 |
| CD45RO PE | | | |
| Mouse Anti-Human | BD Biosciences | H1-100 | 1:20 |
| CD4 APC | | | |
| Mouse Anti-Human | Biolegend | UCHT1 | 1:40 |
| CD45 RA FITC | | | |
| Mouse Anti-Human | | | |
| CD3 BV421 | | | |

### Results

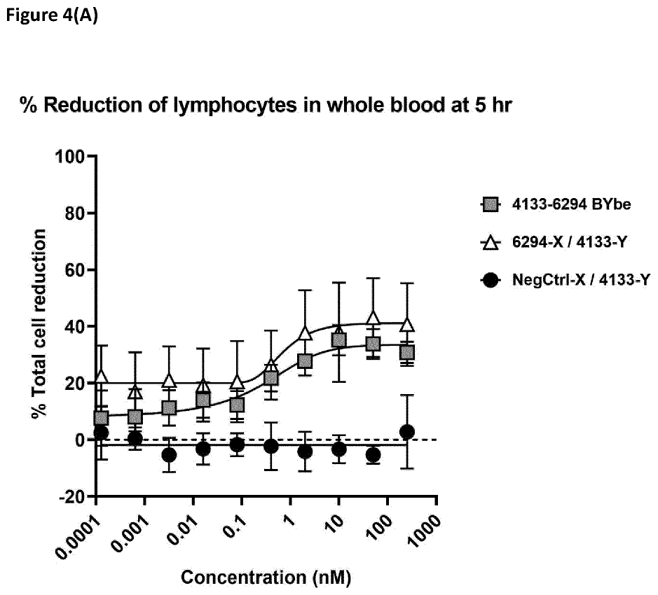

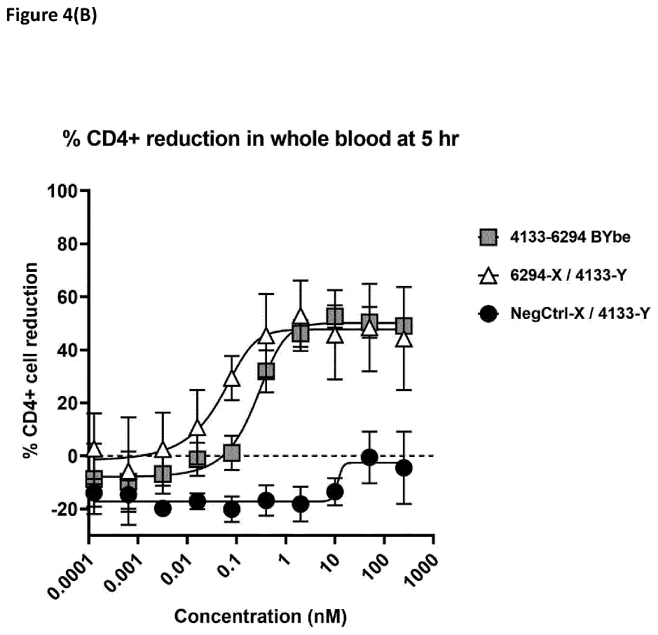

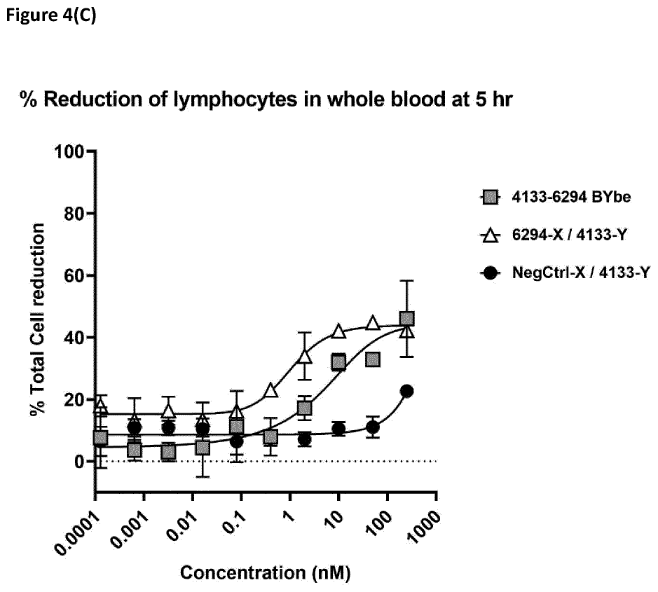

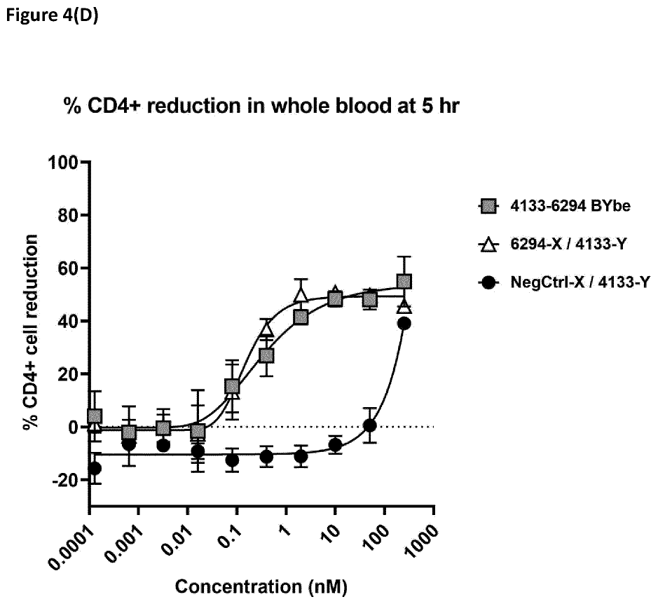

Percentage reductions in whole blood of (A) total lymphocytes and (B) CD⁴⁺ cells (donor #051119-01) and of (C) total lymphocytes and (D) CD⁴⁺ cells (donor #051119-02) by 6294-X/4133-Y and 4133-6294 BYbe are shown in FIG. 4 and Table 8 below. The data is broadly similar across both donors. The negative control, NegCtrl-X/4133-Y, showed no reduction in total lymphocyte or CD4+ cell numbers for either donor. In donor #051119-02, the spike in cell reduction at the highest concentration of NegCtrl-X/4133-Y, is a single datapoint and not thought to reflect true activity. In contrast, in just hrs, both 6294-X/4133-Y and 4133-6294 BYbe showed maximum reductions of total lymphocytes at 34-44% and CD4+ cells at 48-54%. EC50 values for total lymphocytes at 0.37-5.99 nM and for CD4+ cells at 0.05-0.33 nM demonstrated the potency of these reagents and their potential for activity in vivo.

TABLE 8
Reduction in total lymphocyte and CD4+ T cell levels

| | | Donor 1 | | Donor 2 | |
|---|---|---|---|---|---|
| | | | 4133- | | 4133- |
| | | 6294-X/ | 6294 | 6294-X/ | 6294 |
| Cell | Parameter | 4133-Y | BYbe | 4133-Y | BYbe |
| Total | Max % | 41 | 34 | 44 | 44 |
| lymphocytes | reduction | | | | |
| | EC50 (nM) | 0.67 | 0.37 | 1.02 | 5.99 |
| CD4+ | Max % | 48 | 50 | 49 | 54 |
| | reduction | | | | |
| | EC50 (nM) | 0.05 | 0.25 | 0.15 | 0.33 |

### Example 8—Cytokine Release in Whole Blood at 24 Hrs Measured by Luminex Bead Assay

### Method

Human whole blood (Lithium heparin tubes) was taken from two donors (HTA #300120-1 & #300120-2) at UCB Pharma Slough, UK according to approved ethical sample collection protocol.

In a Greiner 96-well non-binding microplate, stocks of 4133-6294 BYbe and negative control BYbe (NegCtrl BYbe), with antigen-irrelevant specificity in both the Fab and scFv positions, were prepared in PBS at 5000 nM. The BYbe reagents were then serially diluted in PBS 1 in 5, three times to produce to form a 4-point dose curve. 12.5 μl of BYbe dilution was transferred into Corning Costar 96-well, cell culture treated, U-shaped-bottom microplate (cat no. 07-200-95) and 237.5 μl of whole blood added to each well. Final well concentrations of BYbes were 250 nm, 50 nM, 10 nm and 2 nM. Campath (clinical grade, diluted from 30 mg/ml stock to 1 mg/ml in PBS, lot number F1002H29) was used as a positive control at a final concentration of 10 μg/ml. Plates were sealed with a gas permeable adhesive seal and plate lids were replaced. Plates were then incubated for 24 hrs at 37° C. and 5% humidified CO₂ in an undisturbed location.

Cytokine release was then assessed using a R&D Systems Luminex 13-plex human cytokine assay (custom selection of cytokines as follows; IL-1 RA, IL-4, IL-5, IL-6, IL-10, IL-11, IL-13, CCL2, IL-8, CXCL1, CX3CL1, GM-CSF and M-CSF). Following the 24-hour incubation, plates were spun at 1000 g for 10 mins and 50 μl of plasma was transferred to a separate plate containing 50 μl of assay diluent buffer (RD6-52 from the Luminex kit). Samples were resuspended thoroughly using a multichannel pipette and 50 μl transferred to the Luminex assay plate. Luminex assay standards were diluted 1 in 2, seven times to construct standard curves and added to the plate. 50 μl of microparticle mixture was then added to each well and plates incubated for 2 hrs at room temperature (RT) and mixed at 800 rpm. Plates were washed 3 times by adding 150 μl of wash buffer to each well then allowing the magnetic beads to bind to a BioTek ELx405 microplate washer magnet before supernatant was aspirated. 50 μl of biotin-antibody cocktail was added to each well at plates incubated for 1 hr at RT with shaking. Plates were then washed as before and a final addition of 50 μl of streptavidin-PE added to each well. Plates were incubated for 30 mins at RT with shaking before a final wash step and addition of 50 μl of wash buffer to each well. The Luminex assay plate was run using the iQUEplus flow cytometer (Sartorius). Standard curves were generated (using provided assay controls) and extrapolated cytokine values generated using Forecyt software (Sartorius). Data was then transferred to Graphpad Prism version 8.1 (Graphpad) to generate data visualisations.

### Results

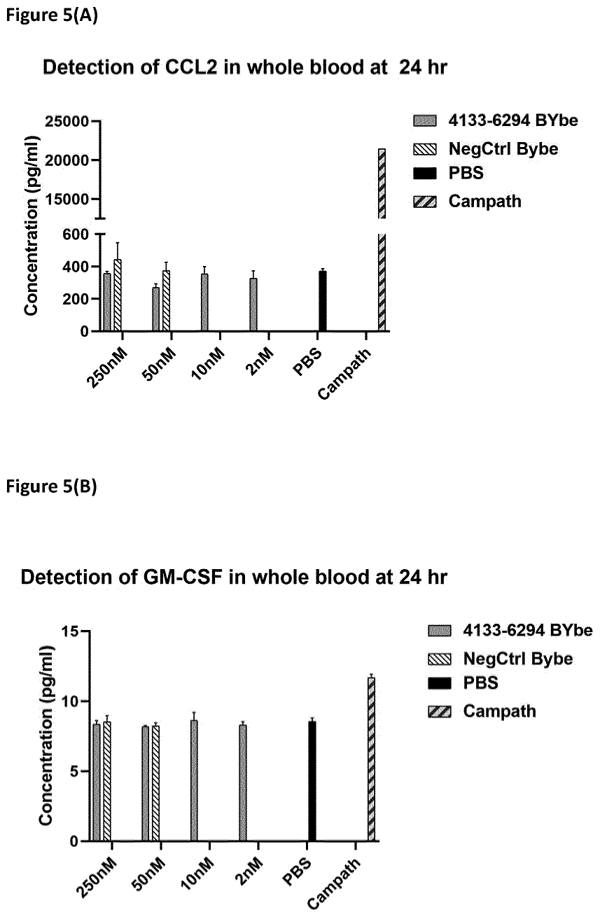

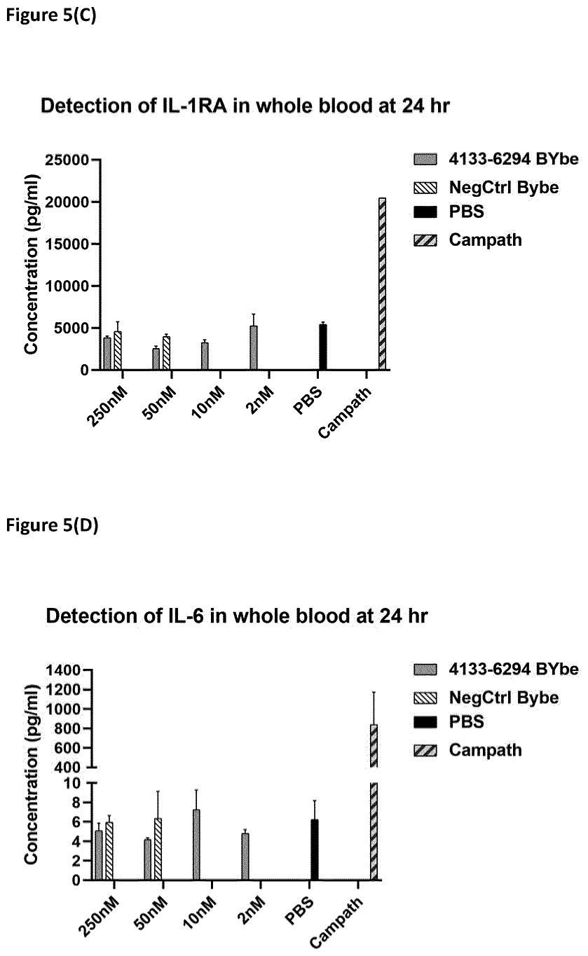

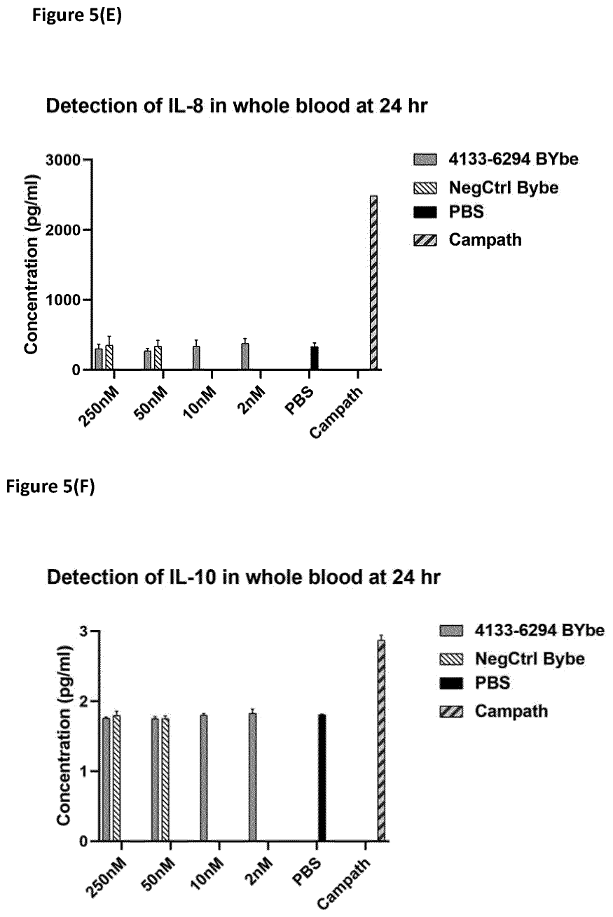

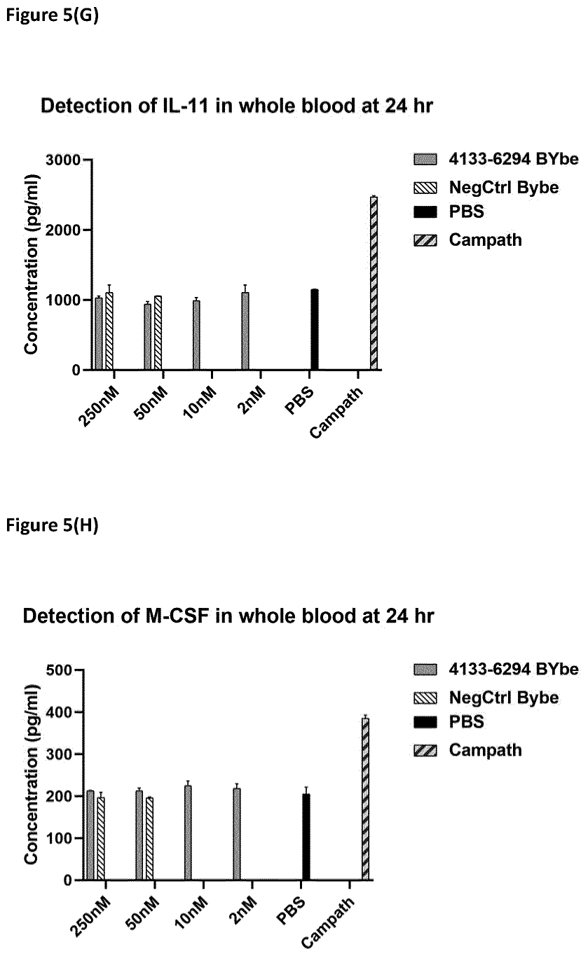

The levels of each cytokine detected in whole blood following incubation for 24 hr with the test reagents was similar across both donors. The data for donor #300120-1 is shown in FIG. 5 as being representative of both donors. The levels of individual cytokines are shown as follows (A) CCL2, (B) GM-CSF, (C) IL-1 RA, (D) IL-6, (E) IL-8, (F) IL-10, (G) IL-11, (H) M-CSF. The cytokines IL-4, IL-5, IL-13, CXCL1 and CX3CL1 could not be detected in any of the wells (data not shown). Campath induced the cytokines (A) CCL2, (C) IL-1 RA, and (E) IL-8 to a level that exceeded the standard curve and therefore have been plotted at the maximum signal in this assay. Campath also induced marked levels of IL-6 (D) above that of PBS-treated wells. Significantly, little or no induction of inflammatory cytokines by 4133-6294 BYbe was observed with the levels matching those in PBS- and NegCtrl BYbe-treated wells.

### Example 9—Cytokine Release in Whole Blood at 24 Hrs Measured by MSD Assay Plus T Cell Count

### Method

Human whole blood (Lithium heparin tubes) was taken from one donor (HTA #031219-06) at UCB Pharma Slough, UK according to approved ethical sample collection protocol.

In a Greiner 96-well non-binding microplate, Fab-X and Fab-Y combination 6294-X/4133-Y was added to PBS to give a Fab-KD-Fab concentration of 2000 nM. Similarly, stocks of 4133-6294 BYbe and NegCtrl BYbe were prepared in PBS at 2000 nM. The microplate was incubated for 1 hr at 37° C., 5% CO₂. Following the incubation, Fab-KD-Fab and BYbe reagents were then serially diluted in PBS 1 in 5, seven times to produce to form an 8-point dose curve.

12.5 μl of Fab-KD-Fab or BYbe dilution was transferred into Corning Costar 96-well, cell culture treated, U-shaped-bottom microplate (cat no. 07-200-95) and 237.5 μl of whole blood added to each well. Final well concentrations of Fab-KD-Fab or BYbes were 100-0.00128 nM. Campath (clinical grade, diluted from 30 mg/ml stock to 1 mg/ml in PBS, lot number F1002H29) was used as a positive control at a final concentration of 10 μg/ml. Plates were sealed with a gas permeable adhesive seal and plate lids were replaced. Plates were then incubated for 24 hrs at 37° C. and 5% humidified CO₂ in an undisturbed location.

Following the 24-hour incubation, plates were spun at 1000 g for 10 mins and 50 μl of plasma was transferred to a separate plate and stored at −80° C. until cytokine release assay. To determine the level of cell depletion, the remaining cells were resuspended in PBS and 50 μl from untreated (PBS), Campath and the Fab-KD-Fab or BYbe 100 nM wells, were transferred to a 96 deep well plate. 950 μl of BD Phosflow BD Lyse/Fix (cat no. BD558049, FisherScientific) was added to each well and the plate incubated at 37° C., 5% CO₂ for 10 minutes. The plate was then spun at 500 g for 8 min, at 4° C. The buffer was aspirated and 1 ml of FACS buffer (PBS+1% bovine serum albumin (BSA)+0.1% NaN₃+2 mM EDTA, Sigma Aldrich) added to wash cells using an Integra Viaflo 96 channel pipette. The plate was spun at 500 g for 8 min, at 4° C. As before, the buffer was aspirated and 1 ml of FACS buffer added to wash cells. This was followed by a slower spin at 250 g for 10 min, at 4° C. Again, buffer was aspirated and 1 ml of FACS buffer added to wash cells. The plate was re-spun at 500 g for 8 min, at 4° C. Buffer was aspirated leaving the cells in a minimal residual volume for cell-specific antibody staining. 20 μl of cell-specific antibody cocktail (shown in Table 9 below) was added to the wells and the plate incubated for 1 hr at 4° C.

Following the incubation with the antibody cocktail, cells were washed twice as outlined above and buffer aspirated to leave the cells in 20 μl residual buffer. 20 μl of FACS buffer was then added to samples to dilute the cells. Cells were analysed live using the Intellicyt iQue Screener PLUS. Live cell counts were extracted as metrics and graphical representations generated using Graphpad Prism version 8.1 (Graphpad). Asymmetric (five parameter) curve fitting was applied.

TABLE 9

| Antibody/Reagent | Supplier | Clone | Dilution |
|---|---|---|---|
| LIVE/DEAD ™ Fixable | Invitrogen | | 1:1000 |
| Near-IR Dead Cell | | | |
| Stain | Invitrogen | UCH-L1 | 1:20 |
| Mouse Anti-Human | BD Biosciences | RPA-T4 | 1:20 |
| CD45RO PE | | | |
| Mouse Anti-Human | BD Biosciences | H1-100 | 1:20 |
| CD4 APC | | | |
| Mouse Anti-Human | Biolegend | UCHT1 | 1:40 |
| CD45 RA FITC | | | |
| Mouse Anti-Human | | | |
| CD3 BV421 | | | |

Measurement of cytokines was carried out using the V-PLEX Human Proinflammatory Panel I (4-Plex) (IFN-γ, IL-1β, IL-6, TNF-α, cat no. K15052D, Meso Scale Discovery) according to manufacturer's instructions. Briefly, the plasma samples were defrosted at RT and diluted 1 in 4 with Diluent 2. 50 μl of sample or standard curve calibrator was added to the Proinflammatory Panel I plates and incubated for 2 hr on a plate shaker at RT. The plates were washed with PBS (supplemented with 0.05% Tween-20) using a BioTek ELx405 microplate washer and 30 μl of detection antibody was added to each well. The plates were incubated for a further 2 h on a plate shaker at RT. The plates were washed as before, and 150 μl of read buffer (diluted 1 in 2 in dH₂O) was added to each well. The plates were then analysed on a SECTOR Imager 6000 (Meso Scale Discovery).

### Results

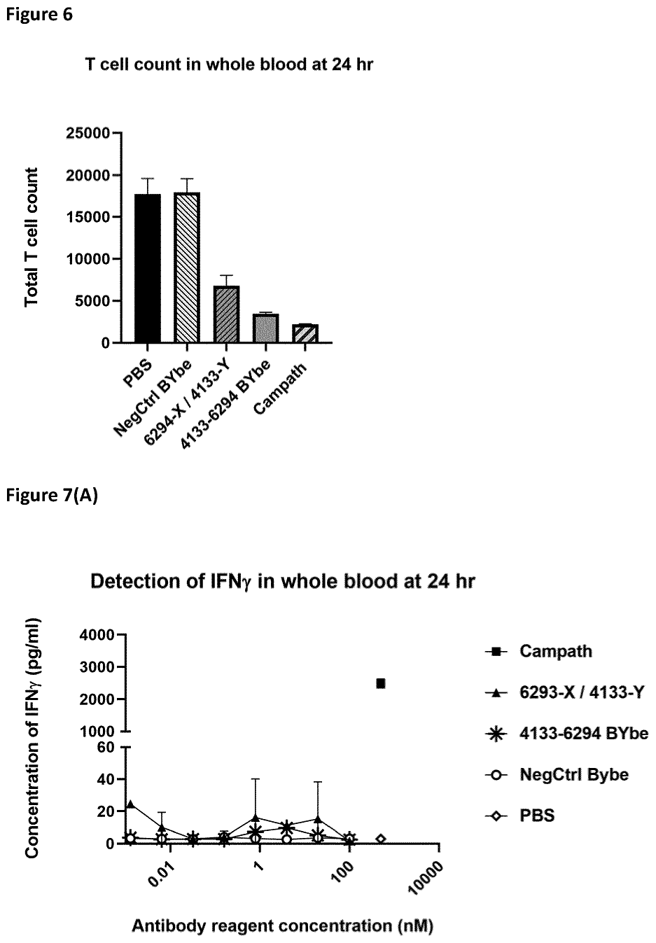

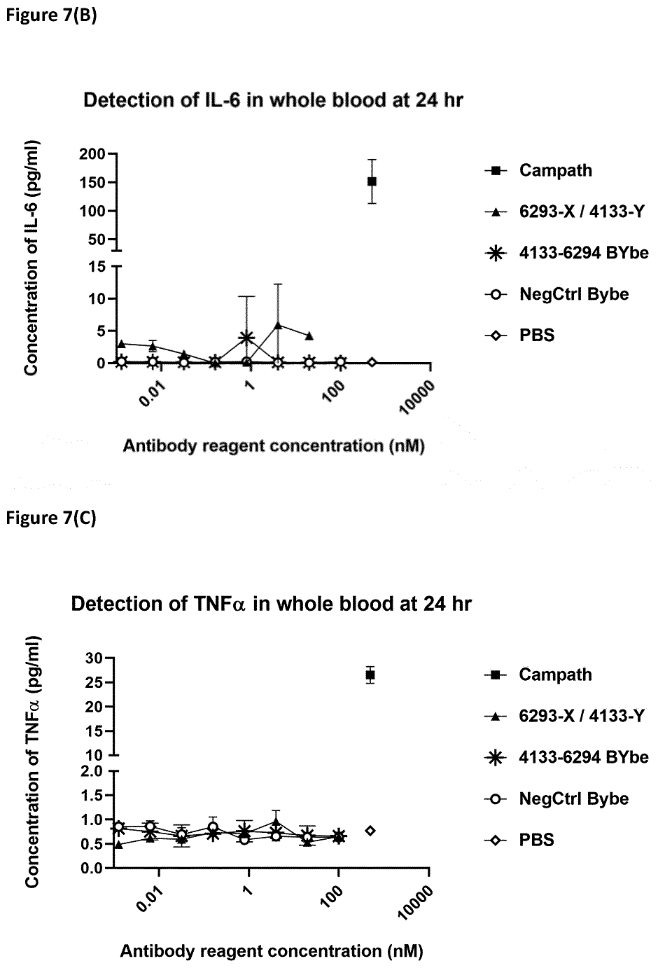

The T cell count in whole blood following incubation for 24 hr with the test reagents is shown in FIG. 6. Campath showed a roughly 8-fold reduction in T cell numbers in comparison with PBS- and NegCtrl BYbe-treated wells. 4133-6294 BYbe and 6294-X/4133-Y also showed a marked reduction in T cell numbers at 5-fold and 3.6-fold, respectively. The levels of inflammatory cytokines detected are shown in FIG. 7 (A) IFN-γ, (B) IL-6 and (C) TNF-α. The levels of IL-1β were below the level of detection for all reagents except Campath, which registered a marked level (data not shown). Significantly, little or no induction of inflammatory cytokines by 4133-6294 BYbe and 6294-X/4133-Y was observed with the levels matching those in PBS- and NegCtrl BYbe-treated wells.

### Example 10—Macrophage Resistance to Apoptosis with Anti-CD45 BYbe

Monocyte Isolation from PBMCs

Human PBMC derived from blood leukocyte platelet-apheresis cones (NHSBT Oxford) were banked as frozen aliquots. Prior to an assay being performed, 2 vials of frozen cells, each containing 5×10⁷ cells in 1 ml, were thawed in a 37° C. water bath and then added to 50 ml complete media (RPMI 1640+2 mM GlutaMAX+1% Pen/Strep, all previous supplied by Invitrogen, +10% Fetal Bovine Serum (FBS), Sigma Aldrich). Cells were spun (500 g, 5 min, at RT) and re-suspended in 30 ml complete media to wash and spun again. Cells were resuspended in 20 ml MACS buffer (PBS, pH 7.2, 0.5% bovine serum albumin (BSA), and 2 mM EDTA, Sigma Aldrich) and counted on a ChemoMetec NucleoCounter NC-3000 to determine concentration and viability. PBMCs from one donor (UCB-Cones 802) was used in this assay.

Monocyte isolation was performed using the Pan Monocyte Isolation Kit (Miltenyi Biotec, cat no. 130-096-537) and LS columns (Miltenyi Biotec, cat no 130-042-401) according to manufacturer's instructions. 100 μl of cells were removed and stored on ice to check monocyte purity post isolation. Isolated cells were stained with BV421 mouse anti-human CD14 (BD Biosciences, cat no. 563743) to check for purity of monocytes using FACS.

Cells were spun at 500 g, 5 min, RT, the buffer was aspirated with a BioTek ELx405 microplate washer, and the cells were re-suspended in FACS buffer (PBS+1% bovine serum albumin (BSA)+0.1% NaN₃+2 mM EDTA, Sigma Aldrich) to wash and then re-spun and buffer was aspirated to leave the cells in 20 μl residual media. 20 μl of the Biotin-Antibody cocktail was added to the wells and the plate incubated for 1 hr at 4° C. Following the incubation, the plate was spun as before, the cells washed once in FACS buffer, spun again as before, and then the excess buffer was aspirated to leave the cells in 50 μl residual buffer.

Live cells were analysed using the Intellicyt iQue Screener PLUS. Live cell counts were extracted as metrics and graphical representations generated using Graphpad Prism version 8.1 (Graphpad).

Derivation of Macrophages from Monocytes

M-CSF (Sigma Aldrich, cat no. SRP3110) and GM-CSF (R&D Systems, cat no. 215-GM/CF) were prepared at 100 μg/ml. Cells were prepared in M1 media (complete media+50 ng/ml GM-CSF) or M2 media (complete media+50 ng/ml M-CSF) at a concentration of 2.5×10⁵ cells/ml. 200 μl cells were plated into two Corning 96 well Black polystyrene microplates (Sigma Aldrich) and incubated at 37° C., 5% CO₂.

Following a 3-day incubation at 37° C., 5% CO₂, the plate was spun (500 g, 5 min, RT), buffer was aspirated, the cells were washed once with PBS and then resuspended in 200 μL of M1 media or M2 media. Following a further 4-day incubation (at 37° C., 5% CO₂), the plate was spun as before, buffer aspirated, the cells washed once with PBS and then resuspended in 150 μl of M1 media (supplemented with 50 ng/ml IFNγ, Sigma Aldrich, cat. no SRP3058) or M2 media (supplemented with 20 ng/ml IL-4, Gibco cat no. PHC0044). The cells were incubated overnight in a 37° C. incubator with 5% CO₂.

Treatment of Macrophages with BYbes

On day 8 post-isolation, in a Greiner 96-well non-binding microplate, 4133-6294-BYbe or NegCtrl BYbe, was added to M1 media or M2 media at a concentration of 4000 nM.

The BYbe proteins were then serially diluted 1 in 5, three times to produce 4 working concentrations. 10 μl of each BYbe dilution was added to the 150 ul of cells in the wells of the 96 well Black polystyrene microplates. The final concentrations in the wells were 250 nM, 50 nM, 10 nM and 2 nM. Camptothecin (cat no. C9911-100MG, Sigma Aldrich) and Staurosporine (cat no. 56942-200UL, Sigma Aldrich) were added as positive controls of apoptosis. Both were diluted in M1 or M2 media and added to the well to produce a final concentration of 5 μM.

One microplate was incubated for a further 24 hours in 37° C. incubator with 5% CO₂ before being used to assess cell viability with CellTiter-Glo®. CellTiter-Glo® Luminescent Cell Viability Assay (Promega, cat no. G9681) was performed according to manufacturer's instructions. 150 μl CellTiter-Glo® was added to the wells and mixed gently on a shaker for 2 min. The plate was then incubated at RT for 10 min, following which 100 μl of the solution from each well was transferred to a Corning™ 96-Well Solid White Polystyrene plate (ThermoFisher). The plate was then read on the BMG Labtech PHERAstar FSX microplate reader using the CellTiter-Glo program.

To the other microplate, on day 8, 10 μl of diluted IncuCyte® Caspase-3/7 Green Apoptosis Assay Reagent (cat no. 4440) and IncuCyte® Cytotox Red Reagent (cat no. 4632) was added (final concentrations 5 μM and 2.5 μM respectively). This was then placed into an IncuCyte® S3 Live-Cell Analysis System, using a 10×objective and imaged every hour for 6 days. The caspase dye was measured with the green laser (350 ms) and the cytotox dye was measured with a red laser (650 ms). The green dye signal was analysed using Incucyte® Zoom2016B. The red cytotox dye signal was poor and therefore analysis was not performed on this channel.

### Results

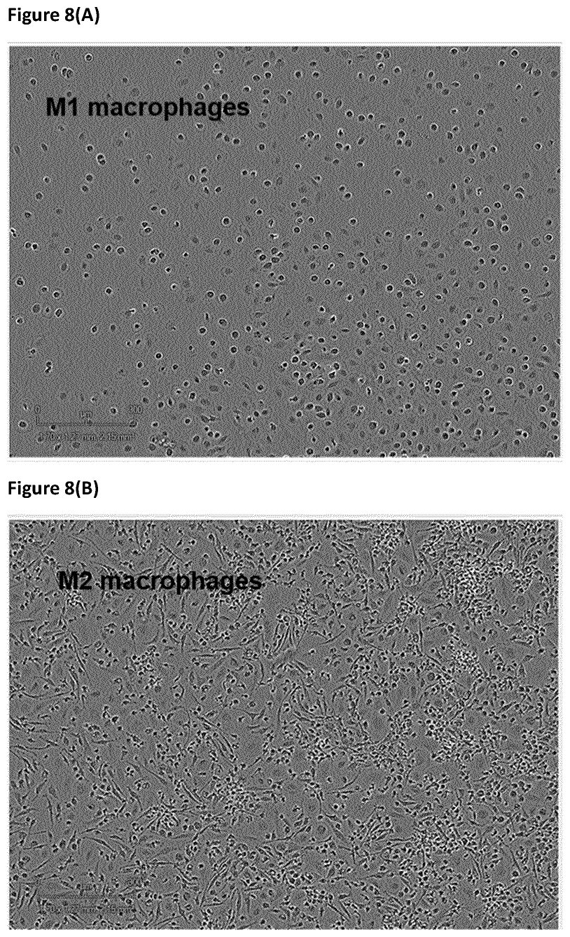

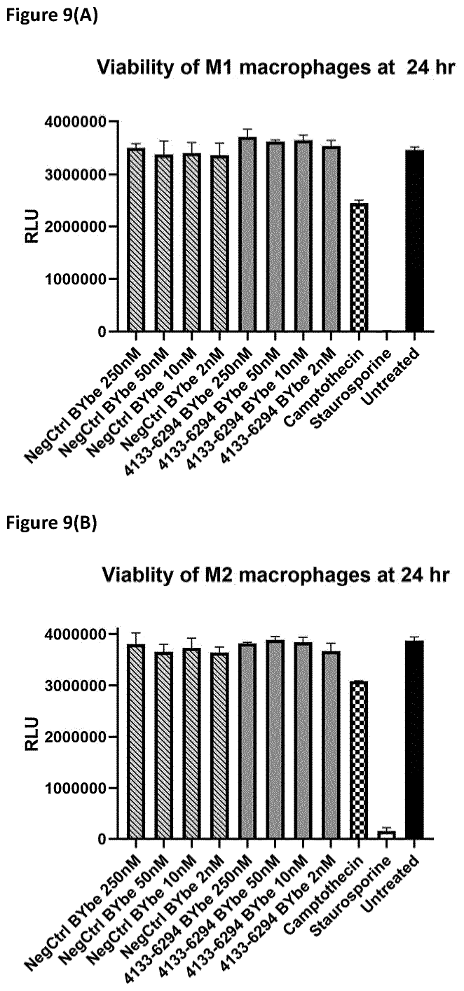

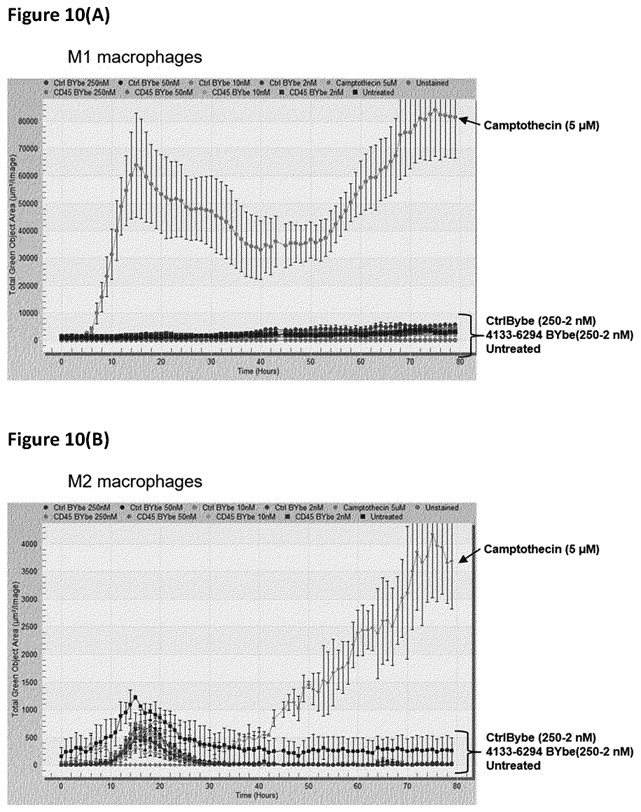

Monocyte-derived macrophages M1 and M2 macrophages looked phenotypically different in (FIG. 8). (A) M1 macrophages were rounded whereas (B) M2 macrophages were more elongated. The M2 macrophages also showed higher confluence than the M1 macrophages, which was expected with M-CSF treatment. Cell viability was assessed at 24 hr to mirror the assay period with PBMCs (FIGS. 3A & 3B). Camptothecin and Staurosporine both reduced the viability of M1 (FIG. 9(A)) and M2 (FIG. 9(B)) macrophages in comparison with untreated cells. The effect of Staurosporine was marked with little or no viable cells detected. In contrast, 4133-6294 BYbe-treated cells showed similar viability to NegCtrl BYbe and untreated wells.

Over the course of 6 days, significant levels Caspase-3/7, could only be detected in Camptothecin-treated macrophages (FIGS. 10(A) & (10B)). The signal in Staurosporine-treated macrophages was very low and thus excluded from both plots. The levels of Caspase-3/7 in 4133-6294 BYbe-treated M1 and M2 macrophages were in-line with NegCtrl BYbe-treated and untreated macrophages. This indicated that macrophages are largely resistant to 4133-6294 BYbe-induced apoptosis. At roughly 16 hrs, M2 macrophages showed a small peak in Caspase-3/7 levels with all treatments. This was thought to be due stress induced by high cell density.

### Example 11—Mass Photometry

### Method

Data were acquired on a RefeynOneMP mass photometer (Refeyn Ltd, Oxford, UK) using AcquireMP (Refeyn Ltd, v2.2.1) software and images were processed and analysed using DiscoverMP (v2.3.dev12) software.

Measurements were performed using clean glass coverslips (High Precision coverslips, No. 1.5, 24×50 mm, Marienfeld) mounted with silicon gaskets (CultureWell™ Reusable Gaskets, Grace biolabs) cut in to 2×2 well sections. Protein stocks were diluted directly in Dulbecco's Phosphate-Buffered Saline (DPBS, ThermoFisher). Typical working concentrations of protein complexes were 1-100 nM, depending on the dissociation characteristics of the protein complexes.

The instrument lens was cleaned with Iso-propyl alcohol (IPA), allowed to dry and a drop of Olympus Low Auto Fluorescence Immersion Oil (NC0297589, ThermoFisher) placed on the lens prior to positioning the microscope coverslip with sample in the light stage. To find focus, 15 μl of fresh DPBS was pipetted into a silicone well, the focal position was identified and secured in place with an autofocus system based on total internal reflection for the entire measurement. For each acquisition, 5 μl of diluted protein was introduced into the well, mixed thoroughly (before autofocus stabilization), and movies of 90 s duration recorded. Each sample was measured once, with a new well and buffer used for each measurement. The mixture of CD45 ECD and 4133-6294 BYbe was not pre-incubated and therefore complexing occurred when the 2 proteins were added into the well.

### Results

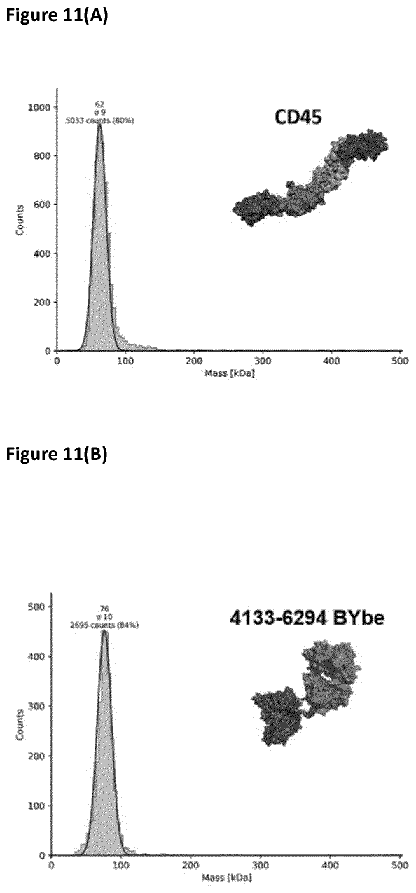

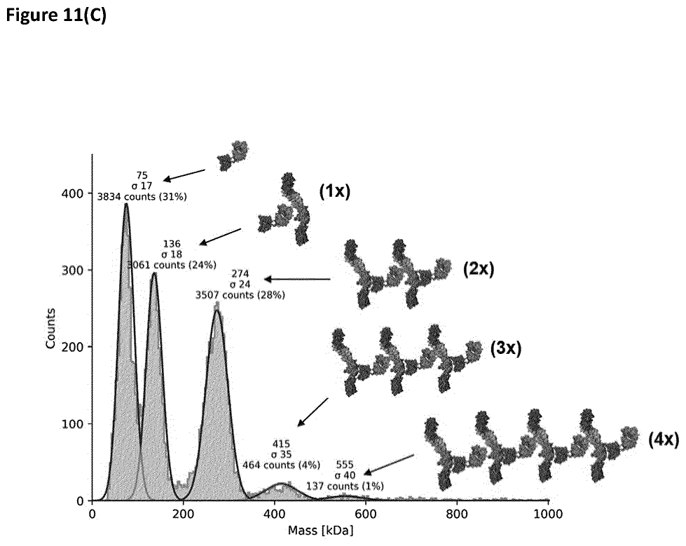

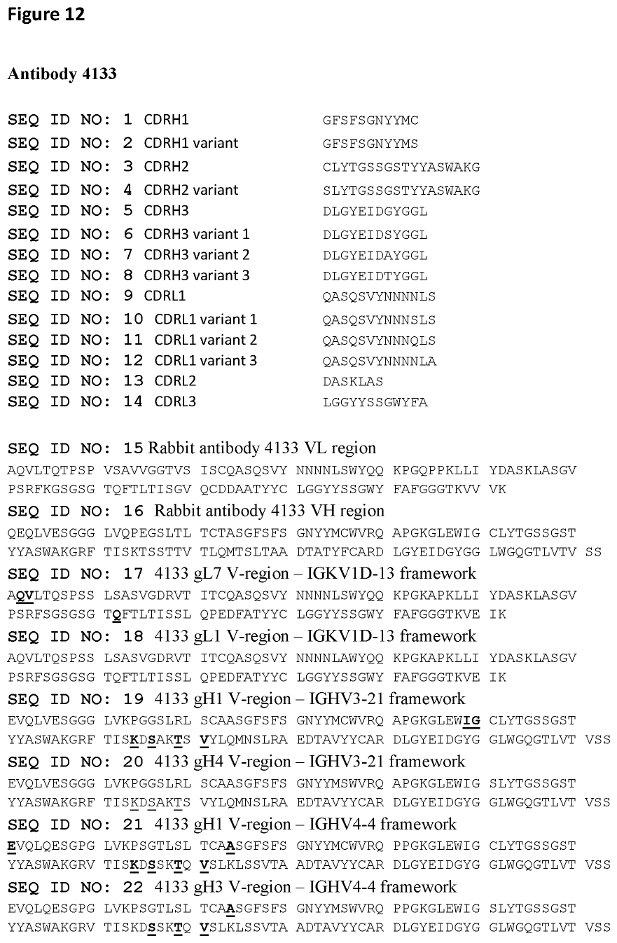

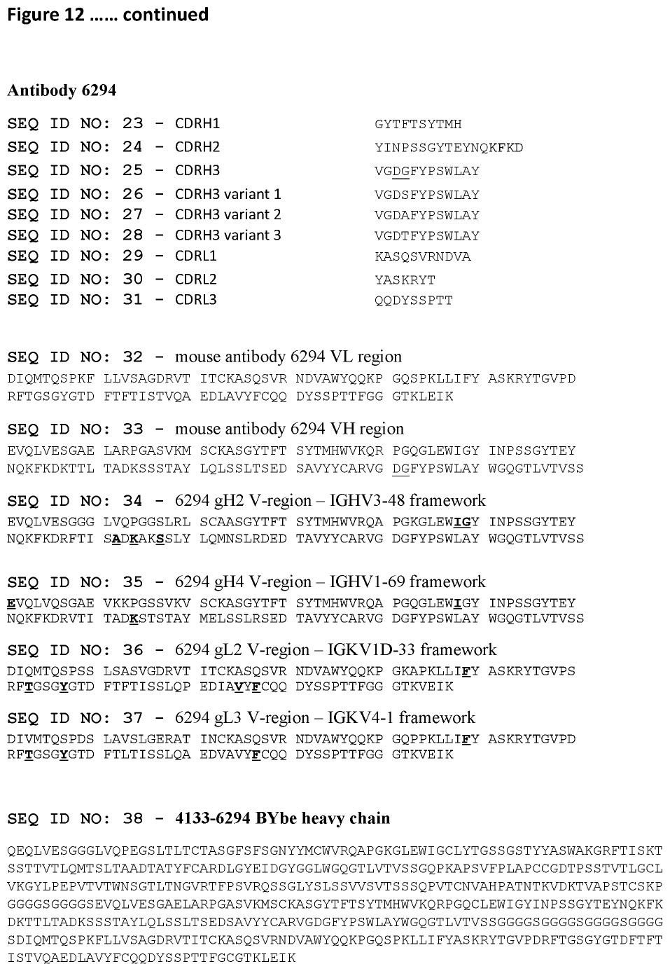

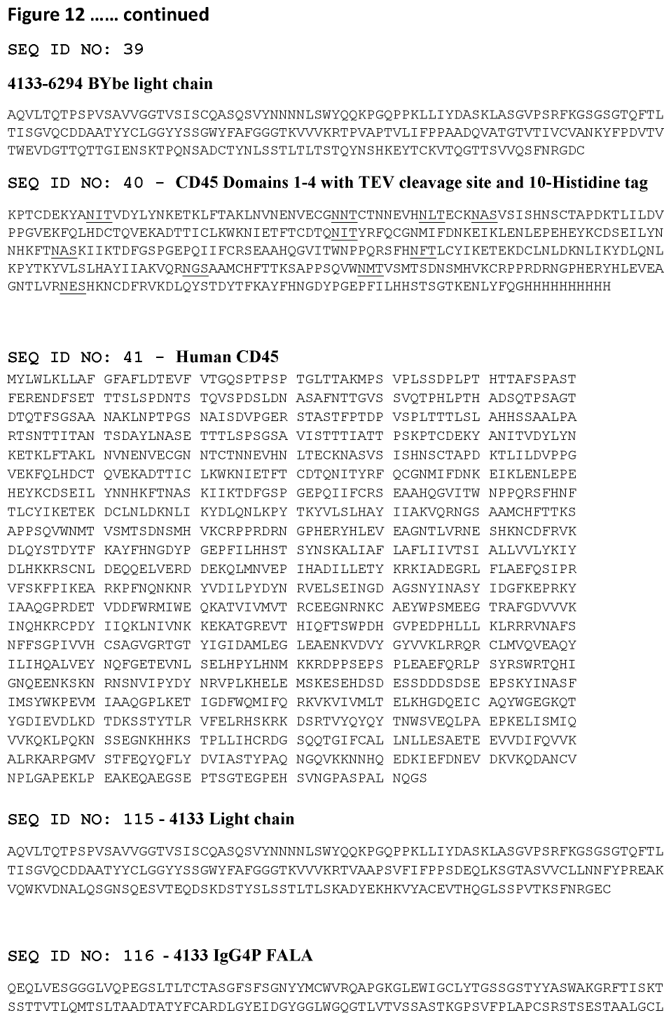

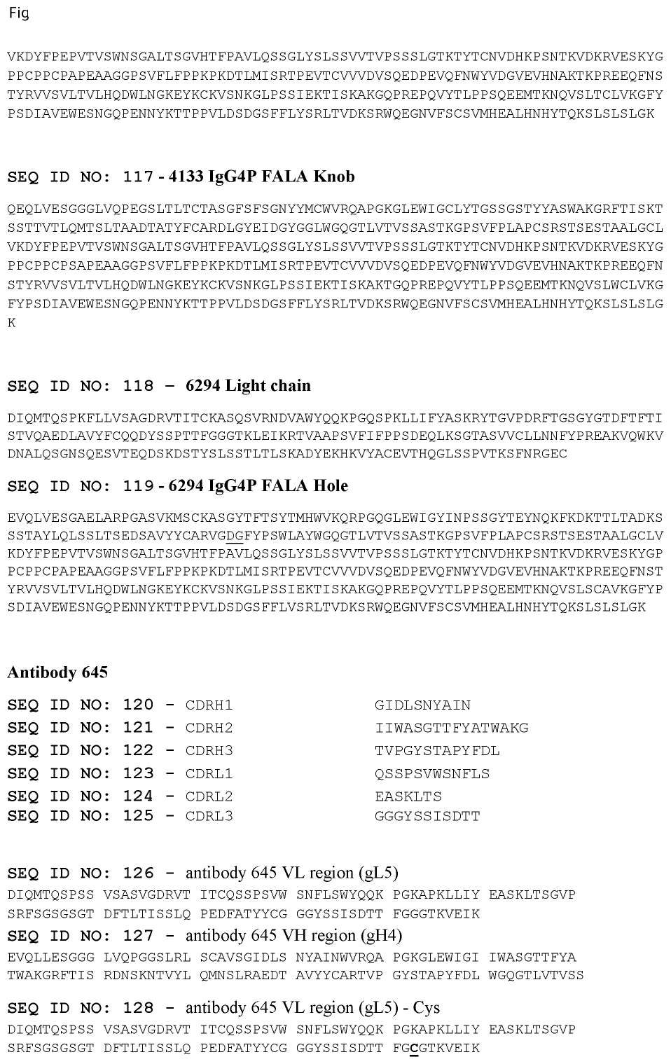

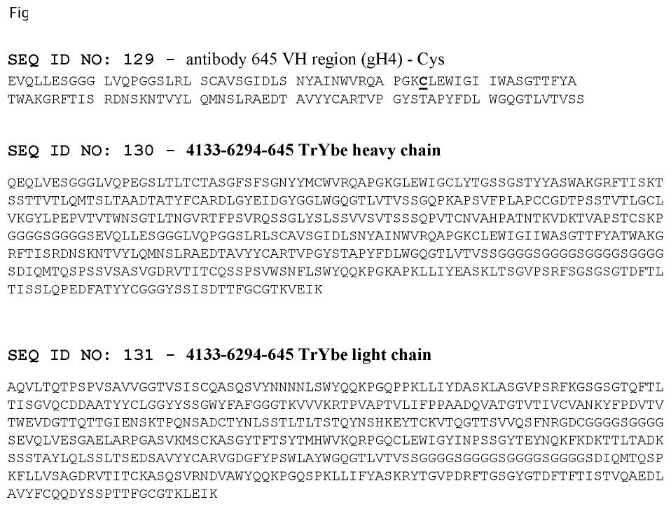

The mass photometry signals for (A) CD45 ECD, (B) 4133-6294 BYbe or (C) a mixture of CD45 ECD and 4133-6294 BYbe are shown in FIG. 11. A single peak was observed for CD45 ECD indicating a homogenous preparation (A). The predicted mass of CD45 ECD is 41.3 kDa. but the peak represented a mass of 62 kDa. It is likely that the difference can be attributed to glycosylation since there are ten predicted N-linked glycosylation sites (see FIG. 12). A single peak corresponding to a mass of 76 kDa was observed for 4133-6294 BYbe (B). This was considered in-line with a predicted mass of 73.5 kDa.

Multiple peaks were observed for the mixture of CD45 ECD and 4133-6294 BYbe (C). The peak at 75 kDa likely corresponds to unbound BYbe. Further peaks were observed at 136, 274, 415 and 555 kDa. The mass of a complex of CD45 ECD and 4133-6294 BYbe is predicted to be 138 kDa based on the observed masses of 62 and 76 kDa, respectively. Thus, the peak at 136 kDa likely corresponds to CD45 ECD-4133-6294 BYbe complex. Furthermore, the peaks at 274, 415 and 555 kDa can likely be assigned to multimeric forms containing 2 copies, 3 copies and 4 copies of the CD45-BYbe complex, respectively (Table 10).

TABLE 10
Theoretical and observed weights
for CD45-BYbe complexes

| | Theoretical | Observed |
|---|---|---|
| Species | mass (kDa) | mass (kDa) |
| 1× (CD45 + BYbe) | 138 | 136 |
| 2× (CD45 + BYbe) | 276 | 274 |
| 3× (CD45 + BYbe) | 414 | 415 |
| 4× (CD45 + BYbe) | 552 | 555 |

### Example 12—Affinities of 4133 and 6294 Fabs as Measured with Surface Plasmon Resonance

### Method

Surface plasmon resonance (SPR) experiments were carried out at 25° C. on a Biacore 3000 system using CM5 sensor chips (GE Healthcare Bio-Sciences AB, Uppsala, Sweden) and HBS-EP running buffer (10 mM HEPES, 150 mM NaCl, EDTA 2 mM and 0.005% (v/v) P20, pH 7.4). 4133 rabbit Fab and 6294 mouse Fab were captured using polyclonal goat F(ab)₂ fragment anti-rabbit F(ab)₂, (Jackson Labs product code #111-006-047) and polyclonal goat F(ab)₂ fragment anti-mouse F(ab)₂, (Jackson Labs product code #115-006-072), respectively. Covalent immobilization of the capturing antibody was achieved by standard amine coupling chemistry to a level of 1000-3000 response units (RU).

CD45 D1-D4 was titrated over the captured purified antibody from 50 nM to 0.05 nM. Each assay cycle consisted of first capturing the antibody Fab fragment using a 1-min injection at a flow rate of 10 μl/min, followed by an association phase consisting of a 3-min injection of CD45 D1-D4 at a flow rate of 30 μl/min. The subsequent dissociation phase was monitored for at least 3 min. After each cycle, the capture surface was regenerated at a flow rate of 10 μl/min with a 1-min injection of 40 mM HCl followed by a 30-sec injection of 5 mM NaOH. A blank flow-cell was used for reference subtraction and buffer-blank injections were included to subtract instrument noise and drift. Kinetic parameters were determined using BIAevaluation software (version 4.1.1).

### Results

The affinities of 4133 and 6294 Fabs were demonstrated to be 61 nM and 85 pM, respectively. The association (Kₐ), dissociation (K_{d}) and affinity (K_{D}) constants are shown in Table 11 below.

TABLE 11
Affinities of 4133 and 6294 Fabs

| | Kₐ (1/Ms) | K_{d} (1/s) | K_{D} (M) |
|---|---|---|---|
| 4133 Fab | 5.7E+05 | 3.5E−02 | 6.1E−08 |
| 6294 Fab | 2.6E+06 | 2.2E−04 | 8.5E−11 |

### Example 13—Humanisation

### Method

Humanised versions of the rabbit antibody 4133 and the mouse antibody 6294 were designed by grafting the CDRs from the donor antibody V-regions onto human germline antibody V-region frameworks. To improve the likelihood of recovering the activity of the antibody, a number of framework residues from the donor V-regions were also retained in the humanised sequences. These residues were selected using the protocol outlined by Adair et al. (1991) (Humanised antibodies. WO91/09967). The CDRs grafted from the donor to the acceptor sequence are as defined by Kabat (Kabat et al., 1987), with the exception of CDRH1 where the combined Chothia/Kabat definition is used (see Adair et al., 1991 Humanised antibodies. WO91/09967). Additionally, the V_{H} genes of rabbit antibodies are commonly shorter than the selected human V_{H} acceptor genes. When aligned with the human acceptor sequences, framework 1 of the V_{H} regions of rabbit antibodies typically lack the N-terminal residue, which is retained in the humanised antibody. Framework 3 of the rabbit antibody V_{H} regions also typically lack one or two residues (75, or 75 and 76) in the loop between beta sheet strands D and E: in the humanised antibodies the gap is filled with the corresponding residues from the selected human acceptor sequence.

The humanised sequences and CDR variants are set out in FIG. 12 and described below.

### CD45 Antibody 4133

Human V-region IGKV1D-13 plus JK4 J-region (IMGT, http://www.imgt.org/) was chosen as the acceptor for antibody 4133 light chain CDRs. In addition to the CDRs, one or more of the following framework residues from the 4133 VK gene (donor residues) may be retained at positions 2, 3 and 70 (Kabat numbering): Glutamine (Q2), Valine (V3) and Glutamine (Q70), respectively. In some cases, CDRL1 may be mutated to remove a potential N-glycosylation site (CDRL1 variant 1-2).

Human V-region IGHV3-21 plus JH1 J-region (IMGT, http://www.imgt.org/) was chosen as an acceptor for the heavy chain CDRs of antibody 4133. In addition to the CDRs, one or more of the following framework residues from the 4133 VH gene (donor residues) may be retained at positions 48, 49, 71, 73, 76 and 78 (Kabat numbering): Isoleucine (148), Glycine (G49), Lysine (K71), Serine (S73), Threonine (T76) and Valine (V78), respectively. In some cases, CDRH1 and CDRH2 may be mutated to remove Cysteine residues (CDRH1 variant and CDRH2 variant, respectively). CDRH3 may also be mutated to modify a potential Aspartic acid isomerisation site (CDRH3 variant 1-3).

Human V-region IGHV4-4 plus JH1 J-region (IMGT, http://www.imgt.org/) was chosen as an alternative acceptor for the heavy chain CDRs of antibody 4133. In addition to the CDRs, one or more of the following framework residues from the 4133 V_{H} gene (donor residues) may be retained at positions 24, 71, 73, 76 and 78 (Kabat numbering): Alanine (A24), Lysine (K71), Serine (S73), Threonine (T76) and Valine (V78), respectively. The Glutamine residue at position 1 of the human framework was replaced with Glutamic acid (E1) to afford the expression and purification of a homogeneous product. In some cases, CDRH1 and CDRH2 may be mutated to remove Cysteine residues (CDRH1 variant and CDRH2 variant, respectively). CDRH3 may also be mutated to modify a potential Aspartic acid isomerisation site (CDRH3 variant 1-3).

CD45 antibody 6294

Human V-region IGKV1D-33 plus JK4 J-region (IMGT, http://www.imgt.org/) was chosen as the acceptor for antibody 6294 light chain CDRs. In addition to the CDRs, one or more of the following framework residues from the 6294 VK gene (donor residues) may be retained at positions 49, 63, 67, 85 and 87 (Kabat numbering): Phenylalanine (F49), Threonine (T63), Tyrosine (Y67), Valine (V85) and Phenylalanine (F87), respectively.

Human V-region IGKV4-1 plus JK4 J-region (IMGT, http://www.imgt.org/) was chosen as the acceptor for antibody 6294 light chain CDRs. In addition to the CDRs, one or more of the following framework residues from the 6294 VK gene (donor residues) may be retained at positions 49, 63, 67 and 87 (Kabat numbering): Phenylalanine (F49), Threonine (T63), and Phenylalanine (F87), respectively.

Human V-region IGHV1-69 plus JH4 J-region (IMGT, http://www.imgt.org/) was chosen as an alternative acceptor for the heavy chain CDRs of antibody 6294. In addition to the CDRs, one or more of the following framework residues from the 6294 VH gene (donor residues) may be retained at positions 1, 48 and 73 (Kabat numbering): Glutamic acid (E1), Isoleucine (148) and Lysine (K73), respectively. In some cases, CDRH3 may be mutated to modify a potential Aspartic acid isomerisation site (CDRH3 variant 1-3).

Human V-region IGHV3-48 plus JH4J-region http://www.imgt.org/) was chosen as an acceptor for the heavy chain CDRs of antibody 6294. In addition to the CDRs, one or more of the following framework residues from the 6294 VH gene (donor residues) may be retained at positions 48, 49, 71, 73 and 76 (Kabat numbering): Isoleucine (148), Glycine (G49), Alanine (A71), Lysine (K73), and Serine (S76), respectively. In some cases, CDRH3 may be mutated to modify a potential Aspartic acid isomerisation site (CDRH3 variant 1-3).

### Example 14—Apoptosis of Peripheral Blood Haematopoietic Stem Cells Induced by Anti-CD45 Antibodies

### Method

Human whole blood (K2EDTA tubes) was received from one 18-year-old donor (#PR20T386505, from Cambridge Bioscience, UK). PBMCs were isolated from whole blood using pre-filled LeucoSep tubes (Greiner). Whole blood was layered on to the LeucoSep filter and tubes spun (800 g, 15 min, slow acceleration and deceleration, at RT). The buffy coat was extracted and cells washed twice in sterile PBS. PBMC were resuspended in 50 ml complete media (RPMI 1640+2 mM GlutaMAX+1% Pen/Strep, all previously supplied by Invitrogen, +10% Fetal Bovine Serum (FBS), Sigma Aldrich). Cells were counted using a ChemoMetec NucleoCounter NC-3000. 1×10⁶ cells per well in 80 μl were then added to each well of a Corning Costar 96-well, cell culture treated, U-shaped-bottom microplate (cat no. 07-200-95).

In a Greiner 96-well non-binding microplate, Fab-X and Fab-Y combination 6294-X/4133-Y, was added to complete media to give a Fab-KD-Fab concentration of 1000 nM. Similarly, 1000 nM stocks of 4133-6294 BYbe and NegCtrl BYbe were prepared in complete media. The microplate was incubated for 1 hr at 37° C., 5% CO₂. Following the incubation, Fab-KD-Fab and BYbe reagents were then serially diluted in complete media in a half log dilution series, 7 times to produce an 8-point dose curve.

20 μl of each Fab-KD-Fab or BYbe dilution (final well concentrations 200-0.00632 nM) was added to the cells and incubated for 24 hrs at 37° C., 5% CO₂. Following the incubation, the plate was spun at 500 g, 5 min, RT, the buffer was aspirated with a BioTek ELx405 microplate washer, and the cells were re-suspended in FACS buffer (PBS+1% bovine serum albumin (BSA)+0.1% NaN₃+2 mM EDTA, Sigma Aldrich) to wash and then re-spun and buffer was aspirated to leave the cells in 20 μl residual media. 20 μl of cell-specific marker antibody cocktail solution was added to the wells and incubated for 30 minutes at 4° C. The antibody cocktail is detailed in Table 12 below. The wash and aspiration steps were repeated, leaving the cells in a residual volume of 20 μl. Cell were then stained with a 1 in 1000 dilution of LIVE/DEAD™ Fixable Near-IR Dead Cell Stain (Invitrogen) and incubated for 10 minutes at 4° C. The wash and aspiration steps were repeated, leaving the cells in a residual volume of 20 μl. Cells were fixed by adding 100 μl of BD Cytofix Fixation Buffer (BD Bioscience) for 15 minutes at 4° C. The wash and aspiration steps were repeated and final volume for FACS acquisition adjust to 200 μl per well.

Cells were analysed using the Bio-Rad ZE5 Cell Analyzer. Cell counts were extracted as metrics and graphical representations generated using Graphpad Prism version 8.1 (Graphpad). Haematopoietic stem cells were defined as the lymphocyte lineage negative, CD45 positive and CD34 positive population.

TABLE 12

| Cell-specific antibody cocktail | Supplier | Clone | Dilution |
|---|---|---|---|
| LIVE/DEAD ™ Fixable | Invitrogen | | 1:1000 |
| Near-IR Dead Cell Stain | | | |
| Lineage Markers-APC | | | |
| Mouse anti-human CD3 APC | BD Bioscience | UCHT1 | 1:20 |
| Mouse anti-human CD19 APC | BD Bioscience | HIB19 | 1:20 |
| Mouse anti-human CD14 APC | BD Bioscience | M5E2 | 1:20 |
| Mouse anti-human CD56 APC | BD Bioscience | B159 | 1:20 |
| Mouse anti-human CD45 FITC | BD Bioscience | HI30 | 1:20 |
| Mouse anti-human CD34 BV421 | BD Bioscience | 581 | 1:20 |
| Mouse anti-human CD38 BV605 | BD Bioscience | HB7 | 1:20 |

### Results

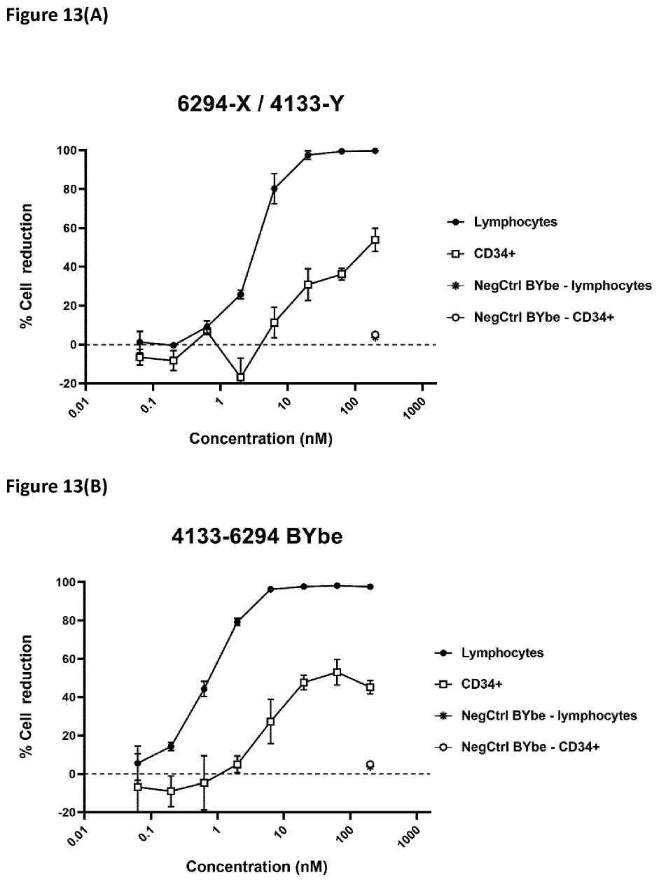

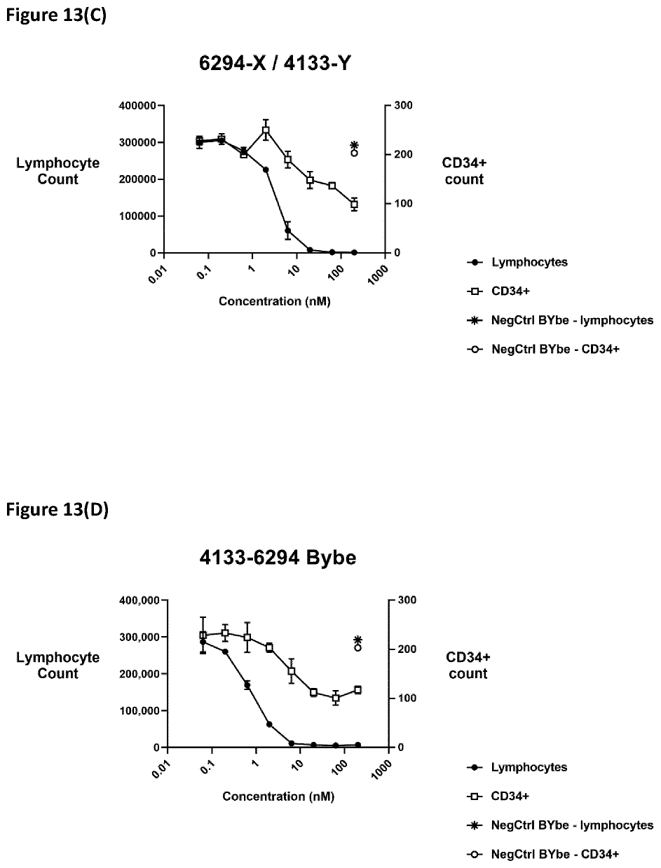

The effect on CD34+ stem cells in PBMCs by 6294-X/4133-Y and 4133-6294 BYbe is shown in FIG. 13. Both reagents showed significant reductions in stem cells at 54% and 53%, respectively ((A) & (B)). In line with previous experiments in PBMCs, reductions in total lymphocytes of 99% and 98%, respectively, were observed ((C) & (D)). It must be noted that the starting number of CD34+ stem cells was roughly 250 in comparison with total lymphocytes at roughly 300,000. This was to be expected since circulating stem cells are known to be present at very low levels.

### Example 15—Sedimentation Velocity

### Method

CD45 ECD and 4133-6294 BYbe were mixed in a molar ratio of 1:1 and incubated for 1 hour at room temperature. The molar ratio was determined using the predicted mass of 4133-6294 BYbe at 73.5 kDa and the mass of CD45 ECD as determined by mass photometry at 62 kDa.

The CD45 ECD-4133-6294 BYbe mixture, CD45 ECD only or 4133-6294 BYbe only were loaded into cells with 2-channel charcoal-epon centrepieces with 12 mm optical path length and glass quartz glass windows. The corresponding buffer was loaded into the reference channel of each cell (the instrument functions like a dual beam spectrometer). Those loaded cells were then placed into an AN-60Ti analytical rotor, loaded into a Beckman-coulter Optima analytical ultracentrifuge and brought to 20° C. The rotor was then brought to 3,000 rpm and the samples were scanned at 280 nm to confirm proper cell loading and appropriate adjustment of the laser, via the laser delay setting. The rotor was then brought to the final run speed of 50,000 rpm. Scans were recorded every 20 seconds for 8 hours. Radial scans ranged from 5.75 to 7.25 cm.

The data were analysed using the c(s) method developed by peter Shuck at the N.I.H and implemented in his analysis program SEDFIT version 14.6e. In this approach many raw data scans directly fitted (36,000 data points for each sample in this case) to derive the distribution of sedimentation coefficients, while modelling the influence of diffusion on the data in order to enhance the resolution. The method works by assigning a diffusion coefficient to each value of sedimentation coefficient based on an assumption that all species have the same overall hydrodynamic shape (with shape defined by the frictional coefficient relative to that for a sphere, f/f0). The f/f0 values were varied to find the best overall fit of the data for each sample. A maximum entropy regularization probability of 0.95 was used and time invariant noise was removed. The analysis was performed using the standard solvent model.

### Results

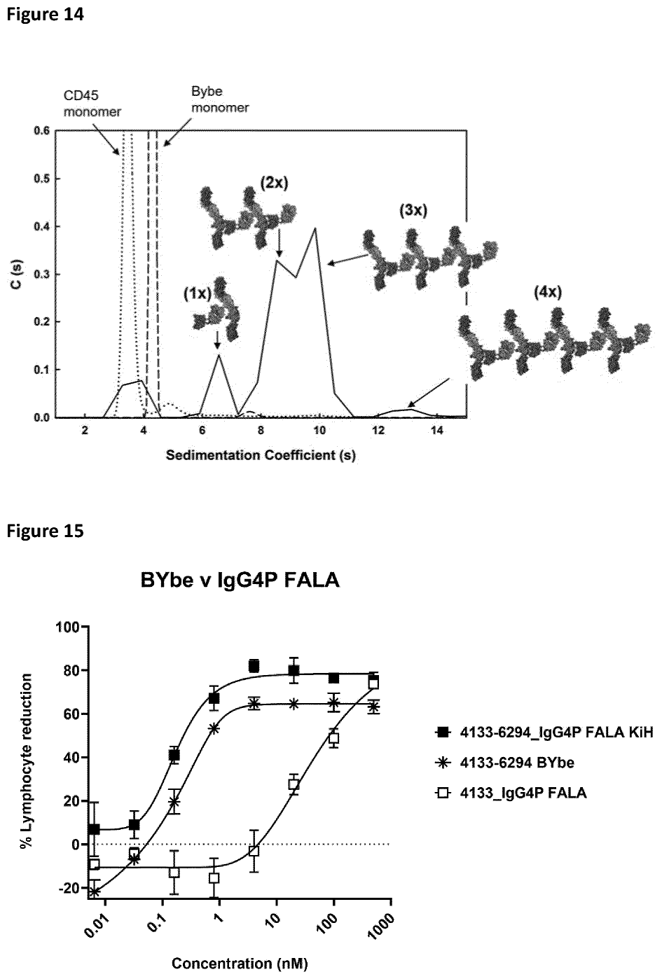

The sedimentation velocities, as measured in an analytical ultracentrifuge, of CD45 ECD monomer, 4133-6294 BYbe monomer and a molar 1:1 mixture of CD45 ECD and 4133-6294 BYbe are shown in FIG. 14. The sedimentation coefficient value of CD45 ECD was 3.547 giving a mass of 58 kDa. This is larger than the predicted mass of CD45 ECD of 41.3 kDa but is in-line with the mass observed by mass photometry (62 kDa) in Example 11. The sedimentation coefficient value of 4133-6294 BYbe was 4.395 giving a mass of 72 kDa. This is in-line with the predicted mass of 73.5 kDa.

Multiple peaks were observed for the mixture of CD45 ECD and 4133-6294 BYbe indicating the presence of CD45 ECD-BYbe multimeric complexes. To assign the complexes, models were made from crystal structures of CD45 ECD (PDB code SFMV) and 4133-6294 BYbe (modelled from individual in-house crystal structures of Fab and scFv) and these were complexed together in a coarse grain manner. Hydrodynamic parameters extracted from these structures revealed the S values calculated from these corresponded to our observed data with an acceptable error (+/−0.5 s). Concluding that we can assign the stoichiometry of the complexes confidently (Table 13).

TABLE 13

| | | Hydrodynamic |
|---|---|---|
| | Experimental | S value |
| Complex | S value | (from modelling) |
| CD45 | 3.6 | 3.7 |
| BYbe | 4.4 | 4.4 |
| CD45 1:1 BYbe | 6.9 | 5.89-6.25 |
| CD45 2:1 BYbe | 7.15 | 7.1-7.2 |
| CD45 2:2 BYbe | 8.78 | 8.43-8.84 |
| CD45 3:3 BYbe | 10.04 | 9.74-10 |
| CD45 4:3 BYbe | 11.12 | 10.7-11.5 |
| CD45 4:4 BYbe | 13.3 | 12.5-13.1 |

Calculation of area under the curve for the trace (Table 14) showed that the mixture is comprised primarily of 2×CD45-BYbe at 37.7% and 3×CD45-BYbe at 35.7%.

TABLE 14

| Complex | Percentage of area under the curve |
|---|---|
| CD45 | 9.21 |
| CD45 1:1 BYbe | 11.99 |
| CD45 2:2 BYbe | 37.7 |
| CD45 3:3 BYbe | 35.7 |
| CD45 4:4 BYbe | 3.46 |
| Higher order than 4:4 | 0.81, 0.54, 0.59 |

### Example 16—Production of IgG4P FALA and IgG4P FALA KiH

### Method

To generate anti-CD45 antibodies 4133 IgG4P FALA and 4133-6294 IgG4P FALA Knob-in-Hole, genes encoding the respective light and heavy chain V-regions of antibodies 4133 and 6294 were designed and constructed by an automated synthesis approach (ATUM). The light V-region genes of antibodies 4133 and 6294 were cloned into an expression vector containing DNA encoding human Cκ region. The heavy V-region gene of antibody 4133 was cloned into expression vectors containing DNA encoding either IgG4P FALA (human IgG4 sequence plus S228P, F234A, L235A) or IgG4P FALA Knob (human IgG4 sequence plus S228P, F234A, L235A, T355W) constant regions. The heavy V-region gene of antibody 6294 was cloned into an expression vector containing DNA encoding IgG4P FALA Hole (human IgG4 sequence plus S228P, F234A, L235A, T366S, L368A, Y407V) constant region.

The 4133 light chain construct was paired with the 4133 IgG4P FALA and 4133 IgG4P FALA Knob heavy chain constructs. The 6294 light chain construct was paired with the 6294 IgG4P FALA Hole construct. The DNA was transfected into CHO-SXE cells using Gibco ExpiFectamine CHO Transfection Kit (cat no. A29133, ThermoFisher Scientific) according to manufacturer's instructions. The cells were cultured for 11 days in an incubator at 32° C., 5% CO₂ with 140 rpm shaking. Following the incubation, cultures were transferred to tubes and supernatant separated from the cells after centrifugation for 2 hours at 4000 rpm. Retained supernatants were filtered through 0.22 μm SARTO BRAN P Millipore followed by 0.22 μm Gamma gold filters.

Antibodies 4133 IgG4P FALA, 4133 IgG4P FALA Knob and 6294 IgG4P FALA Hole were purified from the supernatants using a 5 ml Mab Select Sure column (GE Healthcare) on an AKTA Pure purification system (GE Healthcare Life Sciences) according to manufacturer's instructions. The fractions of eluted protein were combined and concentrated to <5 ml with Amicon Ultra-15 centrifugal filter unit with Ultracel-50 membrane 50 kDa (SigmaAldrich). To obtain a clean fraction, the protein was then passed through a HiLoad Superdex 200 pg 26/60 HPLC size exclusion column on an AKTA Pure purification system. Protein concentration was determined using a Thermo Scientific NanoDrop 2000 (cat no. ND-2000).

To generate 4133-6294 IgG4P FALA Knob-in-Hole, purified 4133 IgG4P FALA Knob and 6294 IgG4P FALA Hole protein were combined in a 1:1 molar ratio, Cysteamine (SigmaAldrich) was added to a final concentration of 5 mM and then the mixture incubated overnight at room temperature. The mixture was then passed through a HiLoad Superdex 200 pg 26/60 HPLC size exclusion column on an AKTA Pure purification system. Protein concentration was determined using a Thermo Scientific NanoDrop 2000 (cat no. ND-2000). Additionally, fractions were analysed using an ACQUITY BEH200 column on a Waters ACQUITY UPLC SEC System and by SDS-PAGE on a 4-20% Tris-glycine gel. Endotoxin was tested for using the Endosafe nexgen-MCS system (Charles River).

### Example 17—Apoptosis of PBMCs Induced by Anti-CD45 Antibodies Formatted as BYbe, IgG4P FALA and IgG4P FALA KiH

### Method

Human PBMC derived from blood leukocyte platelet-apheresis cones (NHSBT Oxford) were banked as frozen aliquots. Prior to an assay being performed, 2 vials of frozen cells, each containing 5×10⁷ cells in 1 ml, were thawed in a 37° C. water bath and then added to 50 ml complete media (RPMI 1640+2 mM GlutaMAX+1% Pen/Strep, all previous supplied by Invitrogen, +10% Fetal Bovine Serum (FBS), Sigma Aldrich). Cells were spun (500 g, 5 min, at RT) and re-suspended in 30 ml complete media to wash and spun again. Cells were resuspended in 10 ml complete media and then counted using a ChemoMetec NucleoCounter NC-3000. 10⁵ cells per well in 80 μl were then added to each well of a Corning Costar 96-well, cell culture treated, U-shaped-bottom microplate (cat no. 07-200-95). The plate was rested in a 37° C., 5% CO₂ incubator for 2 hrs. PBMCs from two donors, UCB-Cones 811 and 831 were used in this assay.

Stocks at 2500 nM of 4133-6294 BYbe, 4133-6294 IgG4P FALA KiH and 4133 IgG4P FALA in complete media were prepared. In a Greiner 96-well non-binding microplate, the reagents were serially diluted in complete media 1 in 5, seven times to produce to form an 8-point dose curve.

20 μl of each dilution (final well concentrations 500-0.0064 nM) was added to the cells and incubated for 24 hrs at 37° C., 5% CO₂. Following the incubation, the plate was spun at 500 g, 5 min, RT, the buffer was aspirated with a BioTek ELx405 microplate washer, and the cells were re-suspended in FACS buffer (PBS+1% bovine serum albumin (BSA)+0.1% NaN₃+2 mM EDTA, Sigma Aldrich) to wash and then re-spun and buffer was aspirated to leave the cells in 20 μl residual media. 20 μl of LIVE/DEAD™ Fixable Near-IR Dead Cell Stain (Invitrogen, 1:1000 dilution) was added to the wells and incubated for 1 hr at 4° C.

Cells were analysed live using the Intellicyt iQue Screener PLUS. Live cell counts were extracted as metrics and graphical representations generated using Graphpad Prism version 8.1 (Graphpad). Asymmetric (five parameter) curve fitting was applied to derive EC50 values.

### Results

The percentage reductions in lymphocytes by 4133-6294 BYbe, 4133-6294 IgG4P FALA KiH and 4133 IgG4P FALA for a representative donor (UCB Cone-811) are shown in FIG. 15. Both 4133-6294 BYbe and 4133-6294 IgG4P FALA KiH showed highly potent EC50's of 0.10 nM and 0.17 nM, respectively. In contrast, the EC50 of 4133 IgG4P FALA was 44 nM.

### Example 18—Apoptosis of PBMCs Induced by a Combination of Anti-CD45 Antibodies

### Method

Human PBMC derived from blood leukocyte platelet-apheresis cones (NHSBT Oxford) were banked as frozen aliquots. Prior to an assay being performed, 2 vials of frozen cells, each containing 5×10⁷ cells in 1 ml, were thawed in a 37° C. water bath and then added to 50 ml complete media (RPMI 1640+2 mM GlutaMAX+1% Pen/Strep, all previous supplied by Invitrogen, +10% Fetal Bovine Serum (FBS), Sigma Aldrich). Cells were spun (500 g, 5 min, at RT) and re-suspended in 30 ml complete media to wash and spun again. Cells were resuspended in 10 ml complete media and then counted using a ChemoMetec NucleoCounter NC-3000. 10⁵ cells per well in 80 μl were then added to each well of a Corning Costar 96-well, cell culture treated, U-shaped-bottom microplate (cat no. 07-200-95). The plate was rested in a 37° C., 5% CO₂ incubator for 2 hrs. PBMCs from two donors, UCB-Cones 811 and 831 were used in this assay.

In a Greiner 96-well non-binding microplate, Fab-X and Fab-Y combination 6294-X/6294-Y, was added to complete media to give a Fab-KD-Fab concentration of 1250 nM. A stock at 1250 nM of 4133 IgG4P FALA in complete media was prepared and combined with 6294-X/6294-Y in an equimolar mix to give a final total antibody concentration of 2500 nM. Stocks at 2500 nM of 4133-6294 BYbe and 4133 IgG4P FALA in complete media were also prepared. In a Greiner 96-well non-binding microplate, the reagents were serially diluted in complete media 1 in 5, seven times to produce to form an 8-point dose curve.

20 μl of each dilution (final well concentrations 500-0.0064 nM) was added to the cells and incubated for 24 hrs at 37° C., 5% CO₂. Following the incubation, the plate was spun at 500 g, 5 min, RT, the buffer was aspirated with a BioTek ELx405 microplate washer, and the cells were re-suspended in FACS buffer (PBS+1% bovine serum albumin (BSA)+0.1% NaN₃+2 mM EDTA, Sigma Aldrich) to wash and then re-spun and buffer was aspirated to leave the cells in 20 μl residual media. 20 μl of LIVE/DEAD™ Fixable Near-IR Dead Cell Stain (Invitrogen, 1:1000 dilution) was added to the wells and incubated for 10 minutes at 4° C.

Cells were analysed live using the Intellicyt iQue Screener PLUS. Live cell counts were extracted as metrics and graphical representations generated using Graphpad Prism version 8.1 (Graphpad). Asymmetric (five parameter) curve fitting was applied to derive EC50 values.

### Results

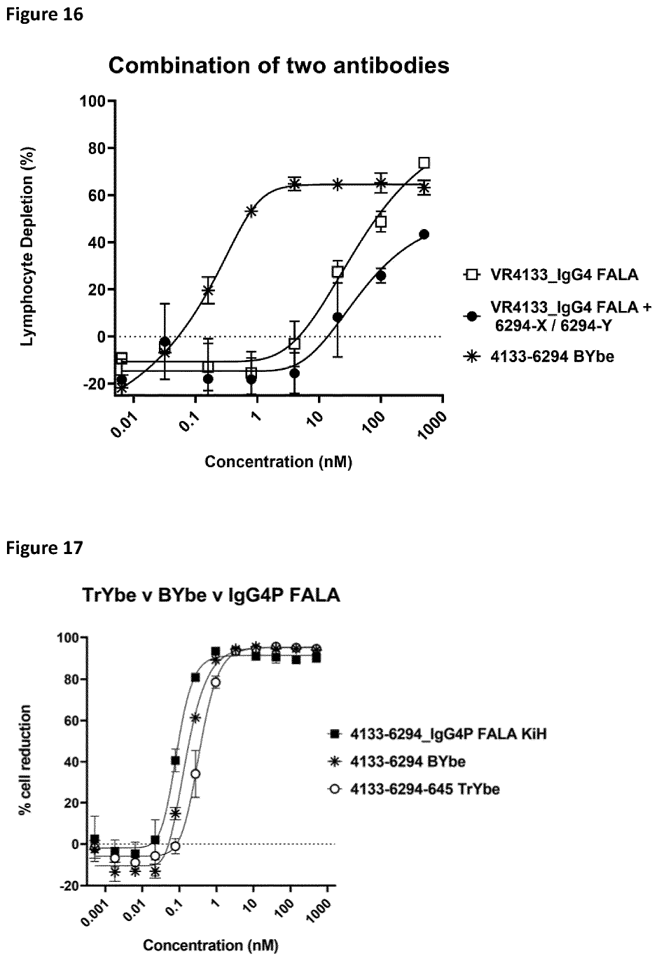

The percentage reductions in lymphocytes by 4133-6294 BYbe, 4133 IgG4P FALA and the combination of 4133 IgG4P FALA for a representative donor (UCB Cone-811) are shown in FIG. 16. 4133-6294 BYbe showed a highly potent EC50 of 0.10 nM. In contrast, the EC50 of 4133 IgG4 FALA was 44 nM. The potency of the combination of 4133 IgG4P FALA and 6294-X/6294-Y was similar to that of 4133 IgG4P FALA alone at 46 nM.

### Example 19—Production of TrYbe

### Method

To generate 4133-6294-645 TrYbe, the full length of the heavy chain (4133 Fab HC-G4S linker-6294 scFv) and the full length of the light chain (4133 Fab LC-G4S linker-645 scFv) were designed and constructed by an automated synthesis approach (ATUM). Both chains were cloned into in-house mammalian expression vectors. 645 binds to human and mouse serum albumin with similar affinity (WO 2011/036460, WO 2010/035012, WO 2013/068571). It confers upon the TrYbe an extended serum half-life.

The heavy and light chain constructs were paired and transfected into CHO-SXE cells using Gibco ExpiFectamine CHO Transfection Kit (cat no. A29133, ThermoFisher Scientific) according to manufacturer's instructions. The cells were cultured for 7 days in an incubator at 37° C., 5% CO₂ with 140 rpm shaking. Following the incubation, cultures were transferred to tubes and supernatant separated from the cells after centrifugation for 30 minutes at 4000 rpm. Retained supernatants were filtered through 0.22 μm SARTO BRAN P Millipore followed by 0.22 μm Gamma gold filters.

The 4133-6294-645 TrYbe protein was purified by native protein G capture step followed by a preparative size exclusion polishing step using an AKTA Pure purification system (GE Healthcare Life Sciences). Clarified supernatants were loaded onto a 50 ml Gammabind Plus Sepharose column (Resin Cytiva, Column packed in house) giving a 25 min contact time and washed with 2.5×column volumes of PBS, pH7.4. Wash fractions with UV readings >25 mAU were collected by fractionation. Bound material was eluted with a 0.1M Glycine pH 2.7 step elution, fractionated and neutralised with 2M Tris/HCl pH 8.5. Both wash material and eluted material were quantified by absorbance at 280 nm.

Size exclusion chromatography (SE-UPLC) was used to determine the purity status of both wash sample and eluted product. The protein (˜3 μg) was loaded on to a BEH200, 200 Å, 1.7 μm, 4.6 mm ID×300 mm column (Waters ACQUITY) and developed with an isocratic gradient of 0.2 M phosphate pH 7 at 0.35 mL/min. Continuous detection was by absorbance at 280 nm and multi-channel fluorescence (FLR) detector (Waters).

The wash fractions and elution fractions containing TrYbe monomer were combined and concentrated using Amicon Ultra-15 concentrator (30 kDa molecular weight cut off membrane) and centrifugation at 4000 g in a swing out rotor. Concentrated samples were applied to a HiLoad 16/600 Superdex 200 pg column (Cytiva) equilibrated in PBS, pH 7.4 and developed with an isocratic gradient of PBS, pH 7.4 at 1 ml/min. Fractions were collected and analysed by size exclusion chromatography on a BEH200, 200 Å, 1.7 μm, 4.6 mm ID×300 mm column (Aquity) and developed with an isocratic gradient of 0.2 M phosphate pH 7 at 0.35 mL/min, with detection by absorbance at 280 nm and multi-channel fluorescence (FLR) detector (Waters). Selected monomer fractions were pooled, 0.22 μm sterile filtered and final samples were assayed for concentration by A280 Scanning on Varian Cary 50 UV Spectrometer (Agilent Technologies). Endotoxin level was less than 1.0 EU/mg as assessed by Charles River's EndoSafe® Portable Test System with Limulus Amebocyte Lysate (LAL) test cartridges.

Monomer status of the final TrYbe was determined by size exclusion chromatography on a BEH200, 200 Å, 1.7 μm, 4.6 mm ID×300 mm column (Aquity) and developed with an isocratic gradient of 0.2 M phosphate pH 7 at 0.35 mL/min, with detection by absorbance at 280 nm and multi-channel fluorescence (FLR) detector (Waters). The final TrYbe antibody was found to be >99% monomeric.

For analysis by sodium dodecyl sulfate-polyacrylamide gel electrophoresis (SDS-PAGE) samples were prepared by adding 4×Novex NuPAGE LDS sample buffer (Life Technologies) and either 10×NuPAGE sample reducing agent (Life Technologies) or 100 mM N-ethylmaleimide (Sigma-Aldrich) to ˜3 μg purified protein, and were heated to 98° C. for 3 min. The samples were loaded onto a 15 well Novex 4-20% Tris-glycine 1.0 mm SDS-polyacrylamide gel (Life Technologies) and separated at a constant voltage of 225 V for 40 min in Tris-glycine SDS running buffer (Life Technologies). Novex Mark12 wide-range protein standards (Life Technologies) were used as standards. The gel was stained with Coomassie Quick Stain (Generon) and destained in distilled water.

### Example 20—Apoptosis of PBMCs Induced by Anti-CD45 Antibodies Formatted as TrYbe, BYbe and IgG4P FALA KiH

### Method

Human PBMC derived from blood leukocyte platelet-apheresis cones (NHSBT Oxford) were banked as frozen aliquots. Prior to an assay being performed, 2 vials of frozen cells, each containing 5×10⁷ cells in 1 ml, were thawed in a 37° C. water bath and then added to 50 ml complete media (RPMI 1640+2 mM GlutaMAX+1% Pen/Strep, all previous supplied by Invitrogen, +10% Fetal Bovine Serum (FBS), Sigma Aldrich). Cells were spun (500 g, 5 min, at RT) and re-suspended in 30 ml complete media to wash and spun again. Cells were resuspended in 10 ml complete media and then counted using a ChemoMetec NucleoCounter NC-3000. 10⁵ cells per well in 100 μl were then added to each well of a Corning Costar 96-well, cell culture treated, U-shaped-bottom microplate (cat no. 07-200-95). The plate was rested in a 37° C., 5% CO₂ incubator for 2 hrs. PBMCs from two donors, UCB-Cones 802 and 812 were used in this assay.

Stocks at 2500 nM of 4133-6294-645 TrYbe, 4133-6294 BYbe and 4133-6294 IgG4P FALA KiH were prepared in complete media. In a Greiner 96-well non-binding microplate, the reagents were then serially diluted in complete media in a 1 in 3.5 dilution series, 11 times to produce a 12-point dose curve. 20 μl of each reagent dilution (final well concentrations 500-0.000518 nM) was added to the cells and incubated for 24 hrs at 37° C., 5% CO₂. Following the incubation, the plate was spun at 500 g, 5 min, RT, the buffer was aspirated with a BioTek ELx405 microplate washer, and the cells were re-suspended in FACS buffer (PBS+1% bovine serum albumin (BSA)+0.1% NaN₃+2 mM EDTA, Sigma Aldrich) to wash and then re-spun and buffer was aspirated to leave the cells in 20 μl residual media. 20 μl of LIVE/DEAD™ Fixable Near-IR Dead Cell Stain (Invitrogen, 1:1000 dilution) was added to the wells and incubated for 10 minutes at 4° C. Cells were analysed using the Bio-Rad ZE5 Cell Analyzer. Cell counts were extracted as metrics and graphical representations generated using Graphpad Prism version 8.1 (Graphpad). Asymmetric (five parameter) curve fitting was applied to derive EC50 values.

### Results

The percentage reductions in lymphocytes by 4133-6294-645 TrYbe, 4133-6294 BYbe and 4133-6294 IgG4P FALA KiH for a representative donor (UCB Cone-802) are shown in FIG. 17. The 4133-6294 TrYbe, 4133-6294 BYbe and 4133-6294 IgG4P FALA KiH were similarly potent with EC50 values of 0.35 nM, 0.15 nM and 0.09 nM, respectively.

### Example 21—Apoptosis of Cell Lines Induced by Anti-CD45 4133-6294 BYbe

### Method

The following cell lines representing various leukaemia's and lymphoma's, as classified by ATCC (www.atcc.org/), were used: Jurkat—acute T-cell leukaemia; CCRF-SB—B-cell acute lymphoblastic leukaemia; MC116—B-cell undifferentiated lymphoma; Raji, Ramos—Burkitt lymphoma (rare form of B-cell non-Hodgkin lymphoma); SU-DHL-4, SU-DHL-5, SU-DHL-8, NU-DUL-1, OCI-Ly3—Diffuse large B-cell lymphoma; THP-1—acute monocytic leukaemia; and Dakiki—B cell nasopharyngeal carcinoma.

Prior to an assay being performed, 1 vial of each of the above cell lines was thawed in a 37° C. water bath and then added to 20 ml complete media (RPMI 1640+2 mM GlutaMAX+1% Pen/Strep, all previous supplied by Invitrogen, +10% Fetal Bovine Serum (FBS), Sigma Aldrich). Cells were spun (500 g, 5 min, at RT) and re-suspended in 20 ml complete media to wash and spun again. Cells were resuspended in 10 ml complete media and then counted using a ChemoMetec NucleoCounter NC-3000. 6×10⁶ cells of each cell line were spun (500 g, 5 min, at RT) and re-suspended in 4.8 ml complete media. 1×10⁵ cells in 80 μl were then added to each well of a Corning Costar 96-well, cell culture treated, U-shaped-bottom microplate (cat no. 07-200-95). The plate was rested in a 37° C., 5% CO₂ incubator for 2 hrs

Stocks of 4133-6294 BYbe and NegCtrl BYbe at 2500 nM in complete media were prepared. In a Greiner 96-well non-binding microplate, both reagents were serially diluted in complete media 1 in 5, seven times to produce to form an 8-point dose curve. μl of each dilution (final well concentrations 500-0.0064 nM) was added to the cells. Each concentration was produced in triplicate. Camptothecin (cat no. C9911-100MG, Sigma Aldrich) and Staurosporine (cat no. 56942-200UL, Sigma Aldrich) were added as positive controls of apoptosis. Both were diluted in complete media and added to the well to produce a final concentration of 5 μM. Additional positive controls included anti-thymocyte globulin (ATG, indicated by FDA for use in conditioning regimens), Rituximab (anti-CD20, indicated by FDA for Non-Hodgkin Lymphoma and Chronic Lymphocytic Leukaemia) and Campath (anti-CD52, indicated by FDA for B-cell Chronic Lymphocytic Leukaemia). Anti-thymocyte globulin, Rituximab and Campath were added to the wells to produce final concentrations of 200 μg/ml, 500 nM and 200 μg/ml, respectively. Each concentration of the controls was produced in triplicate, except for Jurkat with only 2 replicates. The plates were incubated for 21 hrs at 37° C., 5% CO₂.

Following the incubation, the plates were spun at 500 g, 5 min, RT, the buffer was aspirated with a BioTek ELx405 microplate washer, and the cells were re-suspended in FACS buffer (PBS+1% bovine serum albumin (BSA)+0.1% NaN₃+2 mM EDTA, Sigma Aldrich) to wash and then re-spun and buffer was aspirated to leave the cells in 20 μl residual media. 20 μl of LIVE/DEAD™ Fixable Near-IR Dead Cell Stain (Invitrogen, 1:1000 dilution) was added to the wells and incubated for 10 minutes at 4° C. Cells were analysed using the Bio-Rad ZE5 Cell Analyzer. Cell counts were extracted as metrics and graphical representations generated using Graphpad Prism version 8.1 (Graphpad). Asymmetric (five parameter) curve fitting with Constraint Type HillSlope set to “Constant equal to 1” was applied to obtain the best fit and derive maximal reductions in cells and EC50 values.

### Results

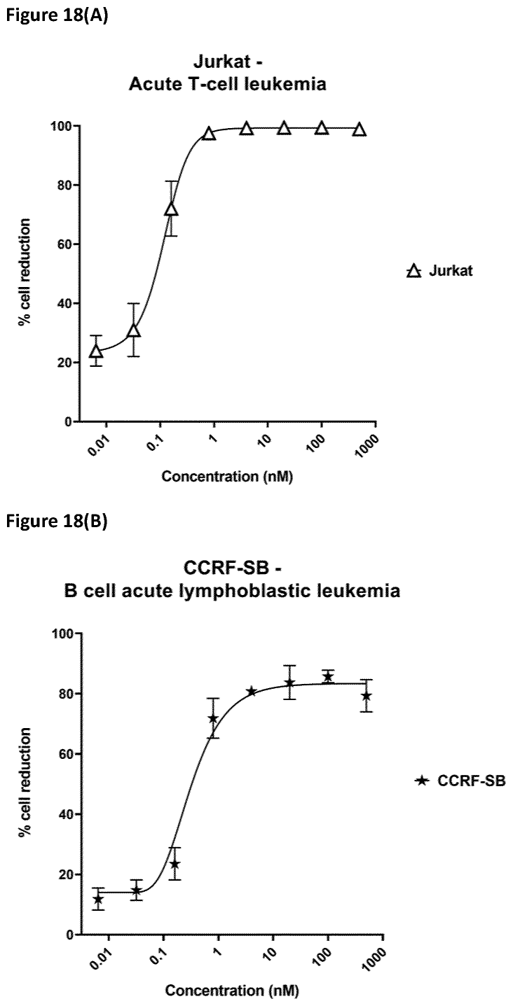

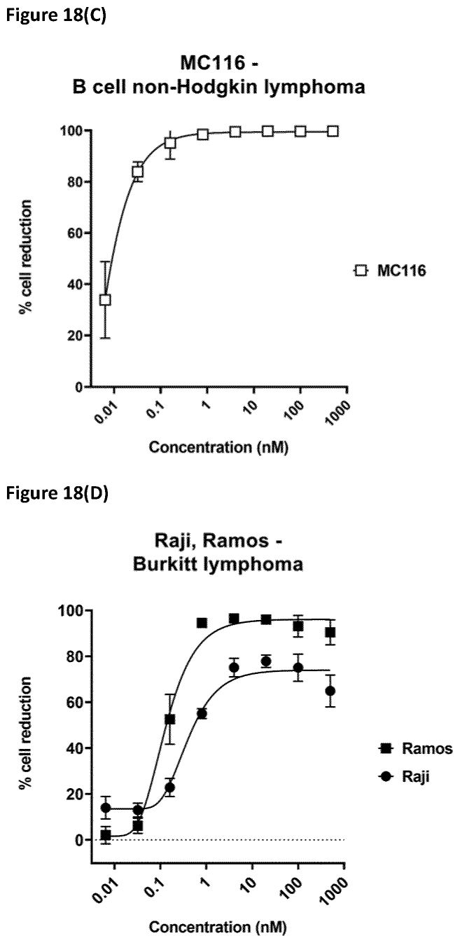

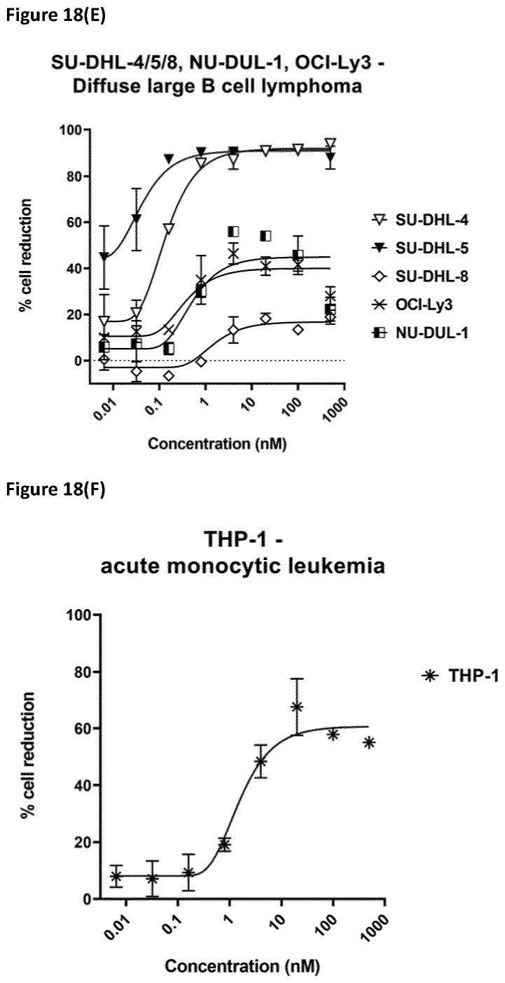

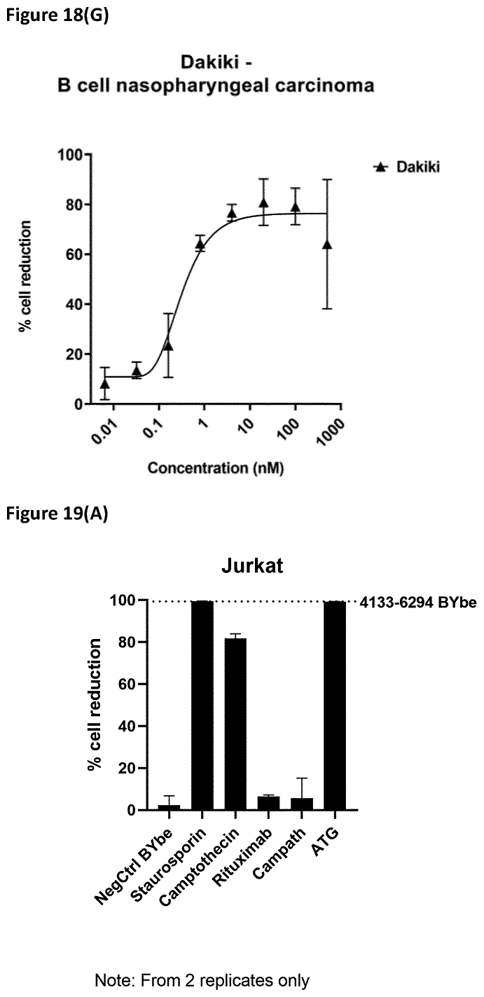

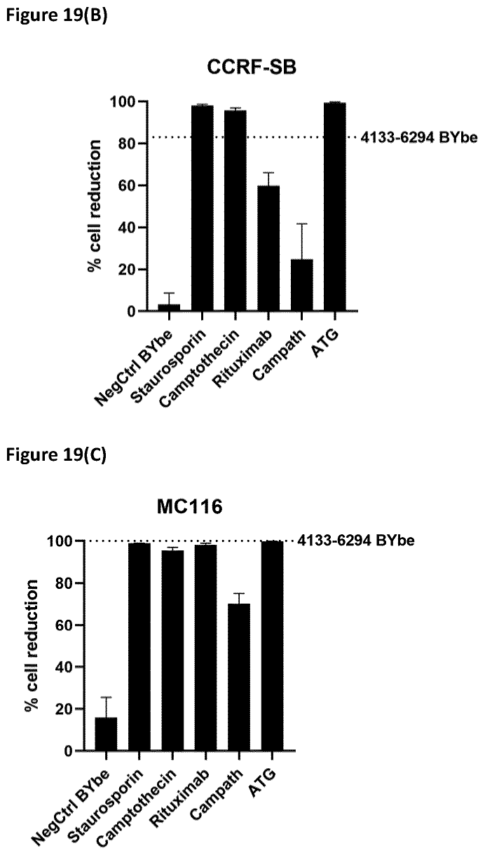

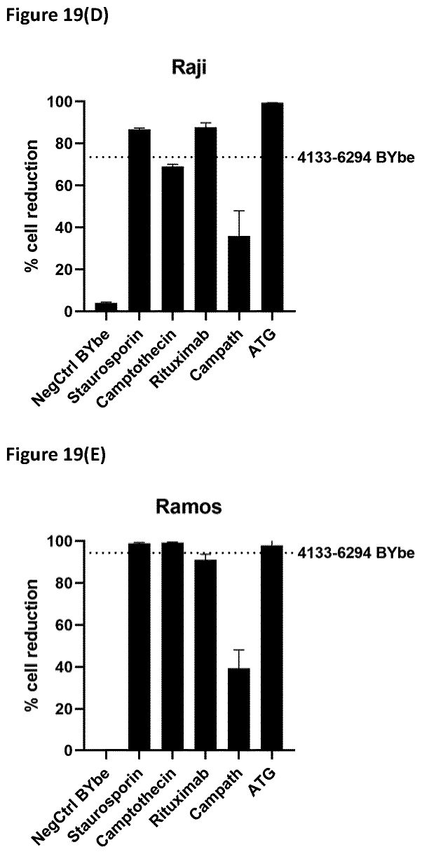

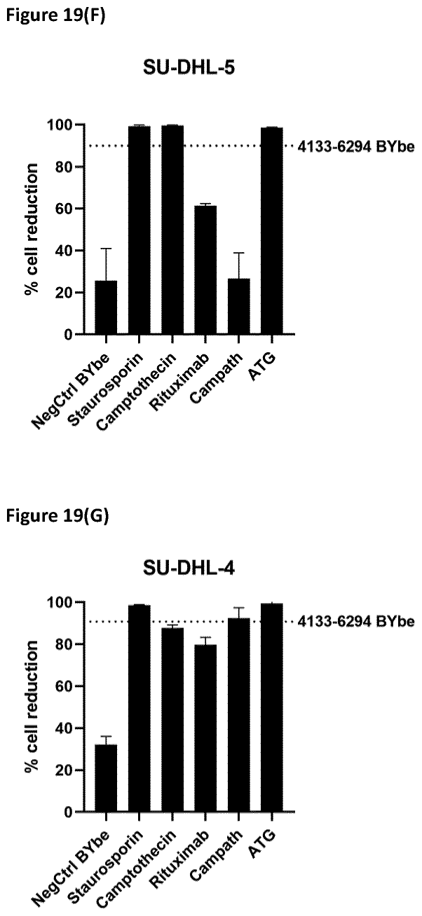

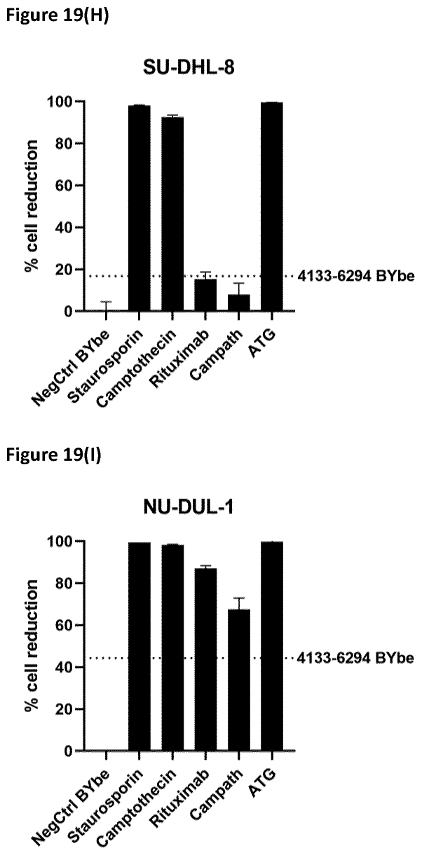

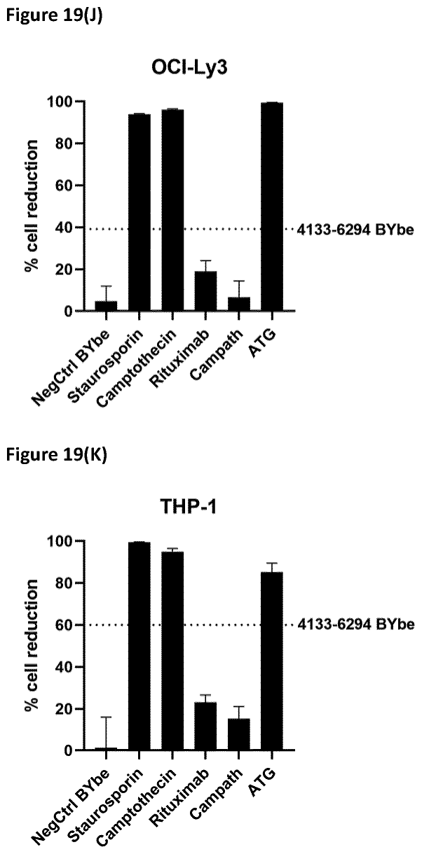

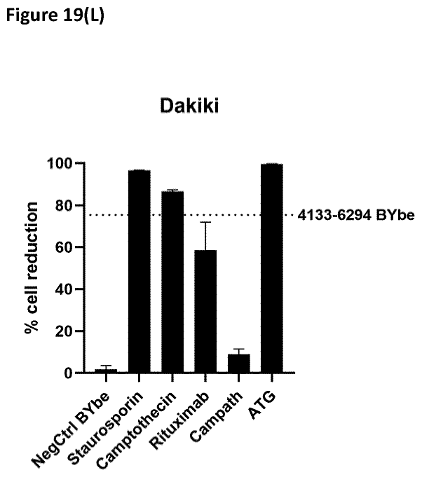

The maximal reductions induced by 4133-6294 BYbe of Jurkat (99.27%), CCRF-SB (83.40%), OCI-Ly3 (39.84%), THP-1 (60.72%) and Dakiki (76.37%) cells were significantly greater than by both Rituximab at 6.52%, 59.87%, 19.05%, 23.12% and 58.55%, respectively, and Campath at 5.64%, 24.86%, 6.72%, 15.40% and 8.83%, respectively (Table 15, FIGS. 18 and 19). The maximal reductions induced by 4133-6294 BYbe of MC116 (99.45%), Raji (74.02%) and Ramos (96.11%) cells were significantly greater than by Campath at 70.24%, 36.00% and 39.40%, respectively, and similar to those by Rituximab at 98.13%, 87.89% and 91.05%, respectively. The maximal reduction induced by 4133-6294 BYbe of SU-DHL-8 (16.77%) was similar to that by both Rituximab at 15.25%, and Campath at 7.97%, respectively. Although 4133-6294 BYbe induced significant reductions of SU-DHL-4 (91.88%) and SU-DHL-5 (91.01%), NegCtrl BYbe also induced reductions of 32.09%, and 25.63%, respectively. The maximal reduction induced by 4133-6294 BYbe of NU-DHL-1 (44.87%) was lower than by both Rituximab at 87.04% and Campath at 67.49%.

TABLE 15
Top and Bottom levels of % cell reduction and EC50 (nM) values for 4133-
6294 BYbe plus mean % cell reductions for NegCtrl BYbe, Rituximab and Campath.
Disease associated with each cell line is also shown: T-cell acute lymphoblastic
leukaemia (T-ALL), B-cell non-Hodgkin lymphoma (NHL), Burkitt lymphoma (BL),
Diffuse large B-cell lymphoma (DLBCL) and Acute monocytic leukaemia (AMOL).

| | | | | | NegCtrl | Rituximab | Campath |
|---|---|---|---|---|---|---|---|
| | | EC50 | Bottom | Top | BYbe mean | mean | mean |
| Cell line | Disease | (nM) | (%) | (%) | (%) | (%) | (%) |
| Jurkat | T-ALL | 0.117 | 23.49 | 99.27 | 2.26 | 6.52 | 5.64 |
| CCRF-SB | B-ALL | 0.308 | 13.95 | 83.40 | 3.23 | 59.87 | 24.86 |
| MC116 | NHL | 0.005 | −65.46* | 99.45 | 16.00 | 98.13 | 70.24 |
| Raji | BL | 0.397 | 13.56 | 74.02 | 4.00 | 87.89 | 36.00 |
| Ramos | BL | 0.140 | 1.61 | 96.11 | 0 | 91.05 | 39.40 |
| SU-DHL-4 | DLBCL | 0.141 | 16.98 | 91.88 | 32.09 | 79.76 | 92.46 |
| SU-DHL-5 | DLBCL | 0.043 | 43.93 | 91.01 | 25.63 | 61.26 | 26.74 |
| SU-DHL-8 | DLBCL | 1.457 | -2.97 | 16.77 | 0.61 | 15.25 | 7.97 |
| NU-DUL-1 | DLBCL | 0.497 | 5.07 | 44.87 | 0 | 87.04 | 67.49 |
| OCI-Ly3 | DLBCL | 0.349 | 10.54 | 39.94 | 4.79 | 19.05 | 6.72 |
| THP-1 | AMoL | 1.545 | 8.14 | 60.72 | 1.17 | 23.12 | 15.40 |
| Dakiki | Carcinoma | 0.309 | 10.88 | 76.37 | 1.72 | 58.55 | 8.83 |

*Although the mean % cell reduction at the lowest concentration is 33.85%, the shape of the curve is not sigmoidal, and this has given rise to a calculated bottom of −65.46%.

1. An antibody comprising at least two different paratopes, each being specific for a different epitope of CD45.

2. An antibody according to claim 1, wherein the antibody is a biparatopic antibody wherein each of the two different paratopes of the antibody is specific for a different epitope of CD45.

3. An antibody according to claim 1 or 2, wherein the CD45 is a human CD45.

4. An antibody according to any one of the preceding claims, wherein the antibody is able to induce cell death of cells expressing CD45, preferably wherein the antibody does not induce the release of cytokines.

5. An antibody according to any one of the preceding claims, wherein the antibody either lacks an Fc region or comprises an Fc region that has been silenced to remove one or more Fc effector functions and/or modified to alter serum pharmacokinetics.

6. An antibody according to any one of the preceding claims, which is selected from a BYbe antibody, a TrYbe antibody, a diabody, a duobody, an IgG (for example an IgG with modifications to promote formation of heterodimers over homodimers and/or purification heterodimers, such as knob-in-hole modifications, charge-charge modifications and/or modification to alter the ability of one heavy chain to bind Protein A or an IgG4(P) antibody with FALA and knob-in-holes modifications).

7. An antibody according to any one of the preceding claims, wherein the antibody is a humanized antibody or a fully human antibody.

8. A nucleic acid molecule or molecules encoding an antibody as defined in any one of the preceding claims.

9. A vector or vectors encoding an antibody as defined in any one of claim 1 to 7 or comprising a nucleic acid molecule or molecules according to claim 8.

10. A pharmaceutical composition comprising:
(a) an antibody according to any one of claims 1 to 7, a nucleic acid molecule or molecules according to claim 8, or a vector or vectors according to claim 9; and
(b) a pharmaceutically acceptable carrier or diluent.

11. A pharmaceutical composition according to claim 10 for use in a method of therapy.

12. A pharmaceutical composition of claim 11 for use in a method of killing disease-associated, CD45-expressing cells in a subject.

13. A pharmaceutical composition of claim 11 or 12 for use in a method of treating a blood cancer, for example leukemia, lymphoma or multiple myeloma.

14. A pharmaceutical composition of claim 11 or 12 for use in a method of treating an autoimmune disease, for example multiple sclerosis or scleroderma.

15. A pharmaceutical composition for use in the method of any one of claims 11 to 14, wherein the method further comprises transferring cells to the subject after the cell depletion.

16. A method of depleting disease-causing, CD45-expressing cells in a subject, the method comprising administering a pharmaceutical composition according to claim 10 to the subject.

17. A method of claim 16 for treating a blood cancer, for example leukemia, lymphoma or multiple myeloma.

18. A method of claim 16 for treating an autoimmune disease, for example multiple sclerosis or scleroderma.

19. A method of any one of claims 16, 17, and 18, wherein the method further comprises transferring cells to the subject after the cell depletion.

20. Use of an antibody according to any one of claims 1 to 7, a nucleic acid molecule or molecules according to claim 8 or a vector or vector according to claim 9 in the manufacture of a medicament for killing disease-associated, CD45-expressing cells in a subject.

21. The use of claim 20 wherein the medicament is for treating a blood cancer, for example leukemia, lymphoma or multiple myeloma.

22. The use of claim 20 wherein the medicament is for treating an autoimmune disease, for example multiple sclerosis or scleroderma.

23. The use of any one of claims 20 to 22, wherein the medicament is for use in a method that further comprises transferring cells to the subject after the cell killing.

24. A binding molecule or molecules that are able to multimerise CD45 to induce cell death of a cell expressing CD45 without also inducing significant cytokine release.

25. The binding molecule or molecules according to claim 24, wherein the binding molecule or molecules are an antibody that specifically binds CD45 or a mixture of at least two different antibodies that specifically bind CD45.

26. The binding molecule or molecules according to claim 25, wherein the antibody or the antibodies of the mixture have an Fc region which is/are modified:
(a) to be an effector optimized Fc region;
(b) to increase formation of heterodimers over homodimers (such as have knob-in-hole modifications);
(c) to have charged residues present that promote the formation of heterodimers over homodimers;
(d) to have altered serum pharmacokinetics and/or
(e) to have altered protein A binding

27. The binding molecule or molecules according to claim 25 or 26, wherein the antibody or the antibodies in the mixture of antibodies have silenced Fc regions.

28. The binding molecule or molecules according to claim 25, wherein the antibody or the antibodies of the mixture of antibodies lack Fc regions.

29. The binding molecule or molecules according to claim 25, wherein the antibody or antibodies of the mixture are selected from a BYbe antibody, a TrYbe antibody, a diabody, a duobody, an IgG, or a knob-in-hole modified IgG, in particular where the antibody or antibodies are IgG4(P) FALA knob-in-hole format.

30. The binding molecule or molecules according to any one claims 24 to 29, wherein:
(a) the binding molecule is an antibody comprising at least two antigen-binding sites having different specificities for CD45; or
(b) the binding molecules are a mixture of antibodies, where collectively the antibodies in the mixture comprise at least two different antigen-binding sites having different specificities for CD45.

31. The binding molecule or molecules according to claim 30, which is a mixture of antibodies, where each antibody has a single specificity for CD45, but the mixture comprises at least two different antibodies having a different specificity for CD45.

32. The binding molecule or molecules according to any one of claims 24 to 31, wherein the antibody or antibodies are chimaeric, humanized or fully human antibodies.

33. The binding molecule or molecules according to any one claims 24 to 32, wherein the antibody or antibodies of the mixture comprise an antigen-binding site specific for serum albumin.

34. A nucleic acid molecule or molecules encoding a binding molecule or molecules as defined in any one of claims 24 to 33.

35. A vector or vectors encoding a binding molecule or molecules as defined in any one of claims 24 to 33 or comprising a nucleic acid molecule or molecules according to claim 34, for instance where the vector is a LNP-mRNA.

36. A pharmaceutical composition comprising:
(a) a binding molecule or molecules according to any one of claims 24 to 33, a nucleic acid molecule or molecules according to claim 34, or a vector or vector according to claim 35; and
(b) a pharmaceutically acceptable carrier or diluent.

37. A pharmaceutical composition according to claim 36 for use in a method of therapy.

38. A pharmaceutical composition according to claim 37 for use in a method of killing disease-associated CD45-expressing cells in a subject.

39. A pharmaceutical composition according to claim 37 or 38 for use in a method of treating a blood cancer, for example leukemia, lymphoma or multiple myeloma.

40. A pharmaceutical composition according to claim 37 or 38 for use in a method of treating an autoimmune disease, for example multiple sclerosis or scleroderma.

41. A pharmaceutical composition according to any one of claim 37 or 38, wherein the method further comprises transferring cells to the subject after the cell killing.

42. A method of killing disease-associated, CD45-expressing cells in a subject, the method comprising administering a pharmaceutical composition according to claim 36 to the subject.

43. The method of claim 42 for treating a blood cancer, for example leukemia, lymphoma or multiple myeloma.

44. The method of claim 42 for treating an autoimmune disease, for example multiple sclerosis or scleroderma.

45. The method of any one of claims 42 to 44, wherein the method further comprises transferring cells to the subject after the cell killing.

46. Use of a binding molecule or molecules according to any one of claims 24 to 33, a nucleic acid molecule or molecules according to claim 34, or a vector or vector according to claim 35 in the manufacture of a medicament for killing disease-associated, CD45-expressing cells in a subject.

47. The use of claim 46 wherein the medicament is for treating a blood cancer, for example leukemia, lymphoma or multiple myeloma.

48. The use of claim 46 wherein the medicament is for treating an autoimmune disease, for example multiple sclerosis or scleroderma.

49. The use of any one of claims 46 to 48, wherein the medicament is for use in a method that further comprises transferring cells to the subject after the cell killing.

50. A method of screening for a binding molecule or molecules able to multimerise CD45 to induce cell death, the method comprising:
(a) contacting a binding molecule or molecules that are able to bind CD45 with target cells expressing CD45; and
(b) determining whether the target cells undergo cell death.

51. The method of claim 50, wherein the method further comprises:
(c) determining whether cytokines are released in the test sample, for example where the level of one or more of CCL2, GM-CSF, IL-1RA, IL-6, IL-8, IL-10, IL-11, and M-CSF is measured.

52. The method of claim 50 or 51, wherein:
(i) the binding molecule or molecules have already been identified as able to multimerise CD45; or
(ii) the method comprises first screening binding molecules specific for CD45 for their ability to multimerise CD45, for example by screening permutations of two or more different binding molecules for their ability to multimerise CD45.

53. An ex vivo method of depleting or killing target cells expressing CD45 in a population of cells, tissue, or organ comprising contacting said cells tissue or organ with an antibody according to any one of claims 1 to 7 or a binding molecule according to any one of claims 24 to 33.

54. An antibody according to any one of claims 1 to 7 or a binding molecule according to any one of claims 24 to 33 for use in a method of treating or preventing graft versus host disease (GVHD) in a subject, the method comprising
(a) contacting ex vivo a population of cells, tissue, or organ with an antibody according to any one of claims 1 to 7 or a binding molecule according to any one of claims 24 to 33 to kill target cells expressing CD45; and
(b) transplanting the treated population of cells, tissue, or organ to said subject.

55. A method of treating or preventing graft versus host disease (GVHD) comprising:
(a) contacting a population of cells, tissue, or organ with an antibody e according to any one of claims 1 to 7 or a binding molecule according to any one of claims 24 to 33 to kill target cells expressing CD45 ex vivo; and
(b) transplanting the treated population of cells, tissue, or organ to a subject in need of such a transplantation.

56. Use of an antibody according to any one of claims 1 to 7 or a binding molecule according to any one of claims 24 to 33 for use in the manufacture of a medicament for treating or preventing graft versus host disease (GVHD) in a method comprising:
(a) contacting a population of cells, tissue, or organ with an antibody according to any one of claims 1 to 7 or a binding molecule according to any one of claims 24 to 33 to kill target cells expressing CD45 ex vivo; and
(b) transplanting the treated population of cells, tissue, or organ to a subject in need of such a transplantation.
